SEQUENCE LISTING (41 sequences)

SEQ ID NO: 1 - DNA, 24 bp, synthetic construct
  source 1..24: mol_type = other DNA
gaaattaatacgactcactatagg

SEQ ID NO: 2 - DNA, 47 bp, synthetic construct
  source 1..47: mol_type = other DNA
cactataggggctctagcgacttctttaaatagtggcttaaaataac

SEQ ID NO: 3 - DNA, 28 bp, synthetic construct
  source 1..28: mol_type = other DNA
gctctagcgacttctttaaatagtggct

SEQ ID NO: 4 - DNA, 68 bp, synthetic construct
  source 1..68: mol_type = other DNA
gaatggaagcaccgagagtatatgaagatcttcatgtgtgcaaaagaatggtaaagcagagaaggagc

SEQ ID NO: 5 - DNA, 29 bp, synthetic construct
  source 1..29: mol_type = other DNA
ttcttttgcacacatgaagatcttcatat

SEQ ID NO: 6 - RNA, 36 nt, synthetic construct
  source 1..36: mol_type = other RNA
gatttagactaccccaaaaacgaaggggactaaaac

SEQ ID NO: 7 - DNA, 28 bp, synthetic construct
  source 1..28: mol_type = other DNA
tcaagctgcaactggccaaaatcccaca

SEQ ID NO: 8 - DNA, 28 bp, unidentified
  source 1..28: mol_type = other DNA
aaaagctgccactggccaaagtcacaca

SEQ ID NO: 9 - DNA, 28 bp, unidentified
  source 1..28: mol_type = other DNA
aagacatgcatctggccaagatcccaca

SEQ ID NO: 10 - DNA, 28 bp, unidentified
  source 1..28: mol_type = other DNA
aaaacctgcacatggccaaaatcacaca

SEQ ID NO: 11 - DNA, 28 bp, unidentified
  source 1..28: mol_type = other DNA
aaaacatgtctgtggcccaagacccata

SEQ ID NO: 12 - DNA, 28 bp, unidentified
  source 1..28: mol_type = other DNA
aaaagctgccattggccaaagtcacaca

SEQ ID NO: 13 - DNA, 28 bp, synthetic construct
  source 1..28: mol_type = other DNA
aaaacctgcaactggccaaaatcccaca

SEQ ID NO: 14 - DNA, 28 bp, synthetic construct
  source 1..28: mol_type = other DNA
aaaacctgccactggccaaaatcacaca

SEQ ID NO: 15 - DNA, 28 bp, synthetic construct
  source 1..28: mol_type = other DNA
tcaggtcctgggaatacatgttggtggg

SEQ ID NO: 16 - DNA, 28 bp, unidentified
  source 1..28: mol_type = other DNA
caaggtactgggtatccatgttggtgga

SEQ ID NO: 17 - DNA, 28 bp, unidentified
  source 1..28: mol_type = other DNA
taaagtccttggaatacatgtcggtggt

SEQ ID NO: 18 - DNA, 28 bp, unidentified
  source 1..28: mol_type = other DNA
gaaagtcctgggaatacacgtaggaggg

SEQ ID NO: 19 - DNA, 28 bp, synthetic construct
  source 1..28: mol_type = other DNA
caaagtcctgggaatacatgttggtggg

SEQ ID NO: 20 - DNA, 28 bp, synthetic construct
  source 1..28: mol_type = other DNA
aaaggtccttggaatacatgttggtgga

SEQ ID NO: 21 - DNA, 28 bp, synthetic construct
  source 1..28: mol_type = other DNA
caaagtccttggaatacatgttggtgga

SEQ ID NO: 22 - DNA, 28 bp, synthetic construct
  source 1..28: mol_type = other DNA
cccacatataggtgccatagaggagaca

SEQ ID NO: 23 - DNA, 28 bp, unidentified
  source 1..28: mol_type = other DNA
tacacgtatcggtgccatagaggagaca

SEQ ID NO: 24 - DNA, 28 bp, unidentified
  source 1..28: mol_type = other DNA
tacacatataggtgtcataggggagaca

SEQ ID NO: 25 - DNA, 28 bp, unidentified
  source 1..28: mol_type = other DNA
tacacatatcggtgtcacagaggagaca

SEQ ID NO: 26 - DNA, 28 bp, unidentified
  source 1..28: mol_type = other DNA
tacacgtaccgatgccacaggggtgata

SEQ ID NO: 27 - DNA, 28 bp, synthetic construct
  source 1..28: mol_type = other DNA
tacacatataggtgccatagaggagaca

SEQ ID NO: 28 - DNA, 28 bp, synthetic construct
  source 1..28: mol_type = other DNA
tccataaatatctctggtcctttgtgag

SEQ ID NO: 29 - DNA, 28 bp, unidentified
  source 1..28: mol_type = other DNA
aatataaatatctctagtcctttgtgag

SEQ ID NO: 30 - DNA, 28 bp, unidentified
  source 1..28: mol_type = other DNA
gatgtaaatatctctggttctctgtgaa

SEQ ID NO: 31 - DNA, 28 bp, unidentified
  source 1..28: mol_type = other DNA
tatatatatatctcttgtcctttgtgac

SEQ ID NO: 32 - DNA, 28 bp, unidentified
  source 1..28: mol_type = other DNA
aatgtaaatgtccctggttctttgtgag

SEQ ID NO: 33 - DNA, 28 bp, unidentified
  source 1..28: mol_type = other DNA
aatataaatgtctctggtcctctgtgac

SEQ ID NO: 34 - DNA, 28 bp, synthetic construct
  source 1..28: mol_type = other DNA
aatataaatatctctggtcctttgtgag

SEQ ID NO: 35 - DNA, 28 bp, synthetic construct
  source 1..28: mol_type = other DNA
tccgtagaagctgttatgtacatgggca

SEQ ID NO: 36 - DNA, 28 bp, unidentified
  source 1..28: mol_type = other DNA
ggtgtagaagctgttatgtacatgggca

SEQ ID NO: 37 - DNA, 28 bp, synthetic construct
  source 1..28: mol_type = other DNA
tgtgtagaagctgttatgtacatgggca

SEQ ID NO: 38 - DNA, 28 bp, synthetic construct
  source 1..28: mol_type = other DNA
actatatgtacttggacagaagtgatgc

SEQ ID NO: 39 - DNA, 28 bp, synthetic construct
  source 1..28: mol_type = other DNA
actatatgtacttggacagacgcaatgc

SEQ ID NO: 40 - DNA, 36 bp, synthetic construct
  source 1..36: mol_type = other DNA
tgggagtgcatactatatgtacttggacagaaacga

SEQ ID NO: 41 - DNA, 36 bp, synthetic construct
  source 1..36: mol_type = other DNA
tgggagtgcatactatatgtacttggacagaagcga

(19) United States
(12) Patent Application Publication
(10) Pub. No.: US 2026/0066050 A1
(43) Pub. Date: Mar. 5, 2026

(54) METHODS AND SYSTEMS TO GENERATE TARGET-BINDING OLIGONUCLEOTIDES

(71) Applicants: President and Fellows of Harvard College, Cambridge, MA (US); Massachusetts Institute of Technology, Cambridge, MA (US)

(72) Inventors: Pardis Sabeti, Cambridge, MA (US); Hayden Metsky, Cambridge, MA (US); Sreekar Mantena, Cambridge, MA (US)

(21) Appl. No.: 19/314,506

(22) Filed: Aug. 29, 2025

Related U.S. Application Data
(60) Provisional application No. 63/689,735, filed on Sep. 1, 2024.

(51) Int. Cl. G16B 40/00; G06F 30/27

(52) U.S. Cl. CPC G16B 40/00; G06F 30/27

(57) ABSTRACT

Current machine learning methods for target binding oligonucleotides design are limited to considering natural sequences in the targets. Here, Applicants generated novel target binding oligonucleotides—with multiple mismatches to any natural sequence—that are optimized for desired properties. These novel target binding oligonucleotides offer more sensitive and specific detection of, for example, pathogen genome variation than baseline design methods, and they illuminate a new, interpretable design rule that broadens nucleic acid sequence targeting.

### CROSS-REFERENCE TO RELATED APPLICATIONS

This application claims the benefit of U.S. Provisional Application No. 63/689,735, filed Sep. 1, 2024. The entire contents of the above-identified applications are hereby fully incorporated herein by reference.

### STATEMENT REGARDING FEDERALLY SPONSORED RESEARCH

This invention was made with government support under Grant No. AI163498 awarded by the National Institutes of Health, and Grant No. D18AC00006 awarded by the Defense Advances Research Projects Agency. The government has certain rights in the invention.

### REFERENCE TO AN ELECTRONIC SEQUENCE LISTING

Reference is made to the electronic sequence listing (“BROD-5820US_ST26.xml”; Size is 36,222 bytes, created on Aug. 4, 2025) is herein incorporated by reference in its entirety.

### TECHNICAL FIELD

The subject matter disclosed herein is generally directed to generating target-binding oligonucleotides with increased sensitivity to target nucleic acid sequences comprising polymorphisms.

### BACKGROUND

Typically, a target-binding oligonucleotide is designed to fully match a particular target nucleic acid sequence. Therefore, to target a particular nucleic acid sequence that may contain one or more polymorphisms, a set of target-binding oligonucleotides must be generated. In many instances, the conclusion is uncertain detection of the target nucleic acid sequences by inefficiently targeting each polymorphism with a unique target-binding oligonucleotide.

Conventional systems are not configured to generate target-binding oligonucleotides comprising one or more mismatches. Typically, conventional systems cannot purposefully introduce asymmetry to a target-binding oligonucleotide and do not facilitate broad nucleic sequence targeting. Conventional systems do not provide solutions for generating multi-target-binding oligonucleotides by introducing one or more mismatches.

Further, conventional systems configure target-binding oligonucleotides based on human assessments of target nucleic acid sequences. Human assessments are unable to introduce one or more mismatches such that the target binding oligonucleotide can effectively target a nucleic acid sequence with polymorphisms. Unlike a machine learning system or artificial intelligence system, systems that rely on humans are unable to draw the subtle conclusions required to introduce effective mismatches. Human systems are unable to create predictive models based on combined data collected from, for example, target nucleic acid sequences for guide nucleic acid sequence, siRNA, primer nucleic acid sequence, or probe nucleic acid sequence.

Citation or identification of any document in this application is not an admission that such a document is available as prior art to the present invention.

### SUMMARY

In an embodiment, the technology described herein includes computer-implemented methods, computer program products, and systems to generate one or more engineered target binding oligonucleotides, comprising: processing one or more target nucleic acid sequences with a deployed oligonucleotide generating network and generating, by the deployed oligonucleotide generating network, one or more engineered target binding oligonucleotides, wherein the one or more engineered target binding oligonucleotides comprise one or more mismatches.

In an embodiment, the method further comprises preparing the one or more engineered target binding oligonucleotides. In an embodiment, the target binding oligonucleotide is a guide nucleic acid sequence, small interfering RNA (siRNA), microRNA (miRNA), diagnostic primer nucleic acid sequence, probe nucleic acid sequence, PNA, or LNA. In an embodiment, the target binding oligonucleotide is a guide nucleic acid sequence. In an embodiment, the mismatch is a nucleotide not complementary to a corresponding nucleotide of at least one, at least 25%, at least 50%, at least all of the target nucleic acid sequences. In an embodiment, two or more mismatches are within a 60-, within a 50-, within a 40-, within a 30-, within a 20-, within a 10-, and within a 5-nucleotide range. In an embodiment, the target binding oligonucleotide comprises a tag adjacent mismatch. In an embodiment, the target oligonucleotide comprises one or more polymorphism. In an embodiment, the mismatch is within 1, 2, 3, 4, 5, 6, 7, 8, 9 or 10 nucleotides from the one or more polymorphisms.

In an embodiment, the oligonucleotide generating network comprises a neural network, Bayesian network, random forest, diffusion model, autoregression model, matrix factorization, hidden Markov model, support vector machine, K-means clustering, K-nearest neighbor, linear classifiers, logistic classifiers, linear regression models, logistic regression models, or any combination thereof. In an embodiment, the neural network comprises a deep learning network, a convolutional neural network, or a recurrent neural network. In an embodiment, the deep learning network comprises a generative adversarial network. In an embodiment, the generative adversarial network comprises a Wasserstein Generative Adversarial Network (WGAN). In an embodiment, the WGAN is conditional on the one or more target nucleic acid sequences.

In an embodiment, the oligonucleotide generating network comprises activation maximization. In an embodiment, the oligonucleotide generating network comprises an evolutionary network. In an embodiment, the evolutionary network introduces one or more mutations to at least one, at least 25%, at least 50%, and at least all target binding oligonucleotides thereby generating a new target binding oligonucleotide. In an embodiment, the one or more mutations occur according to a mutation frequency. In an embodiment, the one or more mutations are random. In an embodiment, the new target binding oligonucleotide is added to the one or more target binding oligonucleotides generating a new set of target binding oligonucleotides and the evolutionary network mutates the new set of target binding oligonucleotides in an iterative process, optionally until a preset number of iterations and/or a preset threshold. In an embodiment, the evolutionary network comprises a fitness evaluation.

In an embodiment, the oligonucleotide generating network comprises an objective network. In an embodiment, the objective network generates a target interaction score between the one or more target binding oligonucleotides and the one or more target nucleic acid sequences. In an embodiment, the objective network generates a non-target interaction score between the one or more target binding oligonucleotides, the one or more target nucleic acid sequences, and one or more non-target sequences. In an embodiment, the objective network comprises a logistic regression model. In an embodiment, the objective network comprises an optimizer. In an embodiment, the method further comprises first training the oligonucleotide generating network with a target interaction score, non-interaction score, or both. In an embodiment, the processing step further comprises processing a target interaction score, non-interaction score, or both and the generating step further comprises generating one or more engineered target binding oligonucleotides with a corresponding target interaction score, non-interaction score, or both. In an embodiment, the method further comprises training the objective network with the one or more engineered target binding oligonucleotides with the corresponding target interaction score, non-interaction score, or both.

In an embodiment, the method further comprises: transmitting the one or more target binding oligonucleotides and the one or more target nucleic acid sequences to a deployed biological activity network, by one or more computing devices; processing the one or more target binding oligonucleotides and the one or more target nucleic acid sequences with the deployed biological activity network; and generating, by the biological activity network, an activity score for the one or more target binding oligonucleotides and the one or more target nucleic acid sequences, wherein the steps are performed any time after the first step in the method.

In an embodiment, the biological activity network comprises a classification network and a regression network. In an embodiment, the classification network generates an active or inactive score. In an embodiment, the regression network generates the level of activity of target binding oligonucleotides. In an embodiment, the activity score is the combination of the classification network and regression network. In an embodiment, the biological activity network comprises a neural network. In an embodiment, the neural network comprises a deep learning network, a convolutional neural network, or a recurrent neural network. In an embodiment, the neural network is a convolutional neural network.

In an embodiment, the oligonucleotide generating network and the biological activity network are deployed from individual training machine learning networks. In an embodiment, the oligonucleotide generating network and the biological activity network are trained using a learning method individually selected from the group consisting of unsupervised learning, supervised learning, semi-supervised learning, reinforcement learning, transfer learning, incremental learning, curriculum learning, learning to learn, contrastive learning, and any combination thereof.

In one aspect, the present disclosure provides a system to generate a engineered target binding oligonucleotides, comprising: a storage device; and a processor communicatively coupled to the storage device, wherein the processor executes application code instructions that are stored in the storage device to cause the system to: process one or more target nucleic acid sequences with a deployed oligonucleotide generating network and generate one or more engineered target binding oligonucleotides with the deployed oligonucleotide generating network, wherein the one or more engineered target binding oligonucleotides comprise one or more mismatches.

In an embodiment, the system further comprises preparing the one or more engineered target binding oligonucleotides. In an embodiment, the target binding oligonucleotide is a guide nucleic acid sequence, SiRNA, diagnostic primer nucleic acid sequence, or probe nucleic acid sequence. In an embodiment, the target binding oligonucleotide is a guide nucleic acid sequence. In an embodiment, the mismatch is a nucleotide not complementary to a nucleotide of at least one, at least 25%, at least 50%, at least all of the target nucleic acid sequences. In an embodiment, two or more mismatches are within a 60-, within a 50-, within a 40-, within a 30-, within a 20-, within a 10-, within a 5-nucleotide range. In an embodiment, the target binding oligonucleotide comprises a tag adjacent mismatch. In example embodiment, the target oligonucleotide comprises one or more polymorphism. In example embodiment, the mismatch is within 1, 2, 3, 4, 5, 6, 7, 8, 9 or 10 nucleotides from the one or more polymorphism.

In an embodiment, wherein the oligonucleotide generating network comprises a neural network, Bayesian network, random forest, diffusion model, autoregression model, matrix factorization, hidden Markov model, support vector machine, K-means clustering, K-nearest neighbor, linear classifiers, logistic classifiers, linear regression models, logistic regression models, or any combination thereof. In an embodiment, the neural network comprises a deep learning network, a convolutional neural network, or a recurrent neural network. In an embodiment, the deep learning network comprises a generative adversarial network. In an embodiment, the generative adversarial network comprises a Wasserstein Generative Adversarial Network (WGAN). In an embodiment, the WGAN is conditional on the one or more target nucleic acid sequences.

In an embodiment, wherein the oligonucleotide generating network comprises activation maximization. In an embodiment, the oligonucleotide generating network comprises an evolutionary network. In an embodiment, the evolutionary network introduces one or more mutations to at least one, at least 25%, at least 50%, at least all target binding oligonucleotide thereby generating a new target binding oligonucleotide. In an embodiment, the one or more mutations occur according to a mutation frequency. In an embodiment, wherein the one or more mutations are random. In an embodiment, the new target binding oligonucleotide is added to the one or more target binding oligonucleotides generating a new set of target binding oligonucleotides and the evolutionary network mutates the new set of target binding oligonucleotides in an iterative process, optionally until a preset number of iterations and/or a preset threshold.

In an embodiment, the evolutionary network comprises a fitness evaluation. In an embodiment, the oligonucleotide generating network comprises an objective network. In an embodiment, the objective network generates a target interaction score between the one or more target binding oligonucleotides and the one or more target nucleic acid sequences. In an embodiment, the objective network generates a non-target interaction score between the one or more target binding oligonucleotides, the one or more target nucleic acid sequences, and one or more non-target sequences. In an embodiment, the objective network comprises a logistic regression model. In an embodiment, the objective network comprises an optimizer. In an embodiment, the system further comprises first training the oligonucleotide generating network with a target interaction score, non-interaction score, or both. In an embodiment, the processing step further comprises processing a target interaction score, non-interaction score, or both and the generating step further comprises generating one or more engineered target binding oligonucleotides with a corresponding target interaction score, non-interaction score, or both. In an embodiment, the system further comprises training the objective network with the one or more engineered target binding oligonucleotides with the corresponding target interaction score, non-interaction score, or both.

In an embodiment, the system further comprises: transmit the one or more target binding oligonucleotides and the one or more target nucleic acid sequences to a deployed biological activity network; process the one or more target binding oligonucleotides and the one or more target nucleic acid sequences with the deployed biological activity network; and generate, by the biological activity network, an activity score for the one or more target binding oligonucleotides and the one or more target nucleic acid sequences, wherein the steps are performed any time after the first step in the system.

In an embodiment, wherein the biological activity network comprises a classification network and regression network. In an embodiment, the classification network generates an active or inactive score. In an embodiment, the regression network generates the level of activity of target binding oligonucleotides. In an embodiment, the activity score is the combination of the classification network and regression network. In an embodiment, the biological activity network comprises a neural network. In an embodiment, the neural network comprises a deep learning network, a convolutional neural network, or a recurrent neural network. In an embodiment, the neural network is a convolutional neural network.

In an embodiment, the oligonucleotide generating network and the biological activity network are deployed from individual training machine learning networks. In an embodiment, the oligonucleotide generating network and the biological activity network are trained using a learning method individually selected from the group consisting of unsupervised learning, supervised learning, semi-supervised learning, reinforcement learning, transfer learning, incremental learning, curriculum learning, learning to learn, contrastive learning, and any combination thereof.

In one aspect, the present disclosure provides a computer program product, comprising: a non-transitory computer-readable storage device having computer-executable program instructions embodied thereon that when executed by a computer cause the computer to generate one or more engineered target binding oligonucleotides, the computer-executable program instructions comprising: computer-executable program instructions to process one or more target nucleic acid sequences with a deployed oligonucleotide generating network and computer-executable program instructions to generate one or more engineered target binding oligonucleotides with the deployed oligonucleotide generating network, wherein the one or more engineered target binding oligonucleotides comprise one or more mismatches.

In an embodiment, the computer program product further comprises preparing the one or more engineered target binding oligonucleotides. In an embodiment, the target binding oligonucleotide is a guide nucleic acid sequence, small interfering RNA (siRNA), microRNA (miRNA), diagnostic primer nucleic acid sequence, probe nucleic acid sequence, PNA, or LNA. In an embodiment, the target binding oligonucleotide is a guide nucleic acid sequence. In an embodiment, the mismatch is a nucleotide not complementary to a nucleotide of at least one, at least 25%, at least 50%, at least all of the target nucleic acid sequences. In an embodiment, the two or more mismatches are within a 60-, within a 50-, within a 40-, within a 30-, within a 20-, within a 10-, within a 5-nucleotide range. In an embodiment, the target binding oligonucleotide comprises a tag adjacent mismatch. In an embodiment, the target oligonucleotide comprises one or more polymorphism. In an embodiment, the mismatch is within 1, 2, 3, 4, 5, 6, 7, 8, 9 or 10 nucleotides from the one or more polymorphism.

In an embodiment, the oligonucleotide generating network comprises a neural network, Bayesian network, random forest, diffusion model, autoregression model, matrix factorization, hidden Markov model, support vector machine, K-means clustering, K-nearest neighbor, linear classifiers, logistic classifiers, linear regression models, logistic regression models, or any combination thereof. In an embodiment, the neural network comprises a deep learning network, a convolutional neural network, or a recurrent neural network. In an embodiment, the deep learning network comprises a generative adversarial network. In an embodiment, the generative adversarial network comprises a Wasserstein Generative Adversarial Network (WGAN). In an embodiment, the WGAN is conditional on the one or more target nucleic acid sequences.

In an embodiment, the oligonucleotide generating network comprises activation maximization. In an embodiment, the oligonucleotide generating network comprises an evolutionary network. In an embodiment, the evolutionary network introduces one or more mutations to at least one, at least 25%, at least 50%, at least all target binding oligonucleotide thereby generating a new target binding oligonucleotide. In an embodiment, the one or more mutations occur according to a mutation frequency. In an embodiment, the one or more mutations are random. In an embodiment, the new target binding oligonucleotide is added to the one or more target binding oligonucleotides generating a new set of target binding oligonucleotides and the evolutionary network mutates the new set of target binding oligonucleotides in an iterative process, optionally until a preset number of iterations and/or a preset threshold.

In an embodiment, the evolutionary network comprises a fitness evaluation. In an embodiment, the oligonucleotide generating network comprises an objective network. In an embodiment, the objective network generates a target interaction score between the one or more target binding oligonucleotides and the one or more target nucleic acid sequences. In an embodiment, the objective network generates a non-target interaction score between the one or more target binding oligonucleotides, the one or more target nucleic acid sequences, and one or more non-target sequences. In an embodiment, the objective network comprises a logistic regression model. In an embodiment, the objective network comprises an optimizer. In an embodiment, the system further comprises first training the oligonucleotide generating network with a target interaction score, non-interaction score, or both. In an embodiment, the processing step further comprises processing a target interaction score, non-interaction score, or both and the generating step further comprises generating one or more engineered target binding oligonucleotides with a corresponding target interaction score, non-interaction score, or both. In an embodiment, the system further comprises training the objective network with the one or more engineered target binding oligonucleotides with the corresponding target interaction score, non-interaction score, or both.

In an embodiment, the computer program product further comprises: transmit the one or more target binding oligonucleotides and the one or more target nucleic acid sequences to a deployed biological activity network; process the one or more target binding oligonucleotides and the one or more target nucleic acid sequences with the deployed biological activity network; and generate, by the biological activity network, an activity score for the one or more target binding oligonucleotides and the one or more target nucleic acid sequences, wherein the steps are performed any time after the first step of the computer program product.

In an embodiment, the biological activity network comprises a classification network and regression network. In an embodiment, the classification network generates an active or inactive score. In an embodiment, the regression network generates the level of activity of target binding oligonucleotides. In an embodiment, the activity score is the combination of the classification network and regression network. In an embodiment, the biological activity network comprises a neural network. In an embodiment, the neural network comprises a deep learning network, a convolutional neural network, or a recurrent neural network. In an embodiment, the neural network is a convolutional neural network.

In an embodiment, the oligonucleotide generating network and the biological activity network are deployed from individual training machine learning networks. In an embodiment, the oligonucleotide generating network and the biological activity network are trained using a learning method individually selected from the group consisting of unsupervised learning, supervised learning, semi-supervised learning, reinforcement learning, transfer learning, incremental learning, curriculum learning, learning to learn, contrastive learning, and any combination thereof.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—A block diagram depicting a portion of a communications and processing architecture of a typical system to acquire one or more target nucleic acid sequences from a user or database and perform machine learning resulting in one or more target binding oligonucleotides, in accordance with certain examples of the technology disclosed herein.

FIG. 2—A block flow diagram depicting methods to generate one or more target binding oligonucleotides, in accordance with certain examples of the technology disclosed herein.

FIG. 3—A block diagram depicting a computing machine and modules, in accordance with certain examples of the technology disclosed herein.

FIG. 4a-4d—Designing optimal guides for two diagnostic objectives using the algorithms in an example embodiment. (4a) Artificial guide sequences—i.e., those that differ from any naturally observed sequence—can increase detection across sequence variation. In the example target set shown, there are two lineages. The consensus of the targets has two mismatches with lineage 2 (in bold), which could reduce enzymatic activity and thus sensitivity for that lineage. The artificial guide sequence, which has only one mismatch with lineage 1 and one with lineage 2, could yield superior performance, since Cas13a better tolerates a single mismatch than two mismatches in close proximity. (4b) Artificial guide sequences can increase the specificity of a guide designed to discriminate one target from another. A baseline approach is to design a guide whose sequence is directly derived from the on-target sequence. Here, the baseline guide has only one mismatch against the off-target sequence, and thus has substantial off-target activity and achieves poor specificity. An artificial guide sequence that has an additional mismatch introduced could have a double mismatch to the off-target sequence, enabling lower off-target activity and more robust specificity. (4c) WGAN-AM algorithm for guide design. The generative model, G(z|T), generates active guide sequences conditional on a given target set T. A latent variable z modulates the generator's output, allowing for different guide sequences. To generate optimal guides, the algorithm starts at a random value of z, computes the fitness of the generated guide f(g|T), and adjusts z in the direction of ∇_{z}f(g|T). (4d) Evolutionary algorithm for guide design. The algorithm initializes a population of candidate guides, G₀, by extracting the guide-length subsequences at the targeted genomic site. To form the population Gₖ in each generation k, n parent guides are sampled from the population Gₖ₋₁ with probability proportional to their fitness f(g|T). The n guides are randomly mutated, and the resulting mutated child guides are added to the population. This process is repeated until L, the limit on the number of guides whose fitness has been evaluated, is reached.

FIG. 5a-5h—Algorithms in in an example embodiment design guides that are maximally sensitive across genomic variation. (5a) Proportion of dengue virus genomes detected by the diagnostic guides designed with methods implemented in an example embodiment (WGAN-AM and evolutionary) and baseline methods (model-based choice (MBC) and consensus). Top, within fixed-size windows along the genome; bottom, within genes and untranslated regions (UTRs). The MBC guides were designed by computing a ground set of sequences and employing the predictive model to select the sequence with the highest fitness. The consensus guides were designed by computing the consensus of the multiple sequence alignment at the targeted genomic site. A guide is considered to detect a target if it meets the criteria described in the Methods. (5b) Relative fitness of the guides designed by MBC, WGAN-AM, and evolutionary algorithms at sites in the dengue virus genome. The relative fitness is the difference between the fitness of the labeled algorithm's guide and the fitness of the consensus guide. A predictive model of guide-target activity was used to compute these fitnesses, and the distribution is across targetable genomic sites (see Methods). P values were computed using one-sided Wilcoxon signed-rank tests. (5c) Minimum Hamming distance between the guides designed by each algorithm and all target sequences at a given genomic site. Distribution is shown across targetable genomic sites. (5d-g) Normalized fluorescence for guides detecting representative targets of genomic sites in influenza A (5d), dengue virus (5e), enterovirus B (5f), and Lassa virus (5g) measured at the one-hour timepoint. The gray shading bar below (5d) and its scale apply to all of (5d-g). Each column represents a target and has width proportional to the percentage of sequence diversity it represents. Each row is a concentration of the target sequence in copies/μL, representing post-amplification target concentrations. (5h) Normalized fluorescence, over the course of the reaction, when detecting the five representative targets of Lassa virus at 10⁸ copies/μL. Parentheticals indicate the percentage of all genomes represented by the target. The MBC-designed guide and consensus guide were identical at this site and are represented by ‘MBC/consensus’.

FIG. 6a-6i—Algorithms in an example embodiment design optimal guides for variant identification and propose a new mechanistic guide design principle. (6a) The objective of the variant identification task is to design a guide sequence that has maximal activity across a set of on-target sequences T₁ and minimal activity across a set of off-target sequences T₂. (6b-d) Design features of 28-nt guides that differentiate between 100 pairs of synthetic targets such that, in each pair, T₂ has a single nucleotide difference (1 SNP) with T₁. For each pair of targets, we applied the algorithms in an example embodiment to design guides for each of the 28 possible SNP placements along the guide, and chose the guide with the highest fitness among the 28 as best able to differentiate the two targets. (6b) Benchmarking predicted activities against a widely-used design approach that introduces a synthetic mismatch between the guide and its targets (Baseline; Methods). Top, distributions of the difference in predicted on-target and off-target activities (A(g|T₁)−A(g|T₂)) across the 100 target pairs. Bottom, break-down of the difference into predicted on-target activity (A(g|T₁); bottom left) and predicted off-target activity (A(g|T₂); bottom right). (6c) Additional guide mismatches were introduced by design algorithms relative to the targets. Namely, plotted value is the percent of—designed guides that have a mismatch against both T₁ and T₂ at positions around the SNP. (Positive positions are 3′ to the SNP; negative are 5′.) (6d) Position of the SNP within the—designed guides. For the majority of target pairs, design algorithms place the SNP within Cas13a's mismatch-sensitive seed region. (6e) Experimental results of guides designed with different approaches for identifying the S139N SNP in Zika virus and A437G antimalarial resistance SNP in P. falciparum at a target concentration of 10⁸ copies/μL. The title of each plot indicates the target the guides were designed to detect as the on-target. (6f) Normalized fluorescence over time of guides designed to identify the K417N/T SNP in SARS-CoV-2 at 10⁸ copies/μL. (6g) Normalized fluorescence over time of guides designed to identify each of the four dengue virus serotypes at 10⁶ copies/μL. (6h) Normalized fluorescence over time of guides designed to identify SARS-CoV-2 lineages at 10⁸ copies/μL. For the Omicron BA.2 on-target, the WGAN-AM and baseline methods designed the same guide (black). For the Ref on-target, the WGAN-AM and evolutionary methods designed the same guide (dark gray). In (6f-h), there are multiple off-targets for each discrimination task, so the dotted off-target curve shows the maximum fluorescence of the guide across the off-targets computed at each time point (e.g., the off-target curves for K417 represent the fluorescence for the higher of the K417N and K417T targets at each time point in the reaction). (6i) Evaluation of the effect of the tag-adjacent mismatch on rescuing guide activity when there are unfavorable (GN) nucleotides at the anti-tag region. Curves show the normalized fluorescence of guides detecting targets that have different nucleotide pairs at the anti-tag region, at 10⁷ copies/μL. All guides are identical to the targets at positions 1-27 in the protospacer. The various gray lines represent guides with a terminal adjacent mismatch (at position 28), while the black dashed lines represent guides without a mismatch to the target. The dinucleotide above each plot indicates the first two nucleotides of that target's anti-tag region, and the guide sequences represented in the schematic are reverse-complemented.

FIG. 7—Pseudocode of the WGAN-AM algorithm, which is further described herein.

FIG. 8—Pseudocode of the evolutionary algorithm. The evolutionary algorithm is further described herein.

FIG. 9a-9e—Coverage of guides designed by the generative design algorithms and baseline methods. Proportion of genomes predicted to be detected by guides designed by different methods. (9a) enterovirus B, (9b) Lassa virus segment S, (9c) influenza A virus segment 2, (9d) SARS-CoV-2, and (9e) dengue virus. A guide is considered to detect a target if it meets the criteria described herein.

FIG. 10a-10b—Relative performance of generative design algorithms. (10a) Relative fitness of the guides designed by the baseline methods and the generative design methods across the five viruses considered for the multi-target detection objective. For each distribution, the point represents the median, lines above and below the point represent the 10th percentile and 90th percentile, respectively, across targetable genomic sites (see Methods). The relative fitness is the difference between the fitness of the labeled algorithm's guide and the fitness of the consensus guide. All the generative design methods outperformed baseline approaches; across these 5 viruses, the design algorithms achieved an average relative fitness ((1/total #of sites across all viruses)

$${\sum }_{i = 1}^{5 viruses} {\sum }_{j = 1}^{\# of sites in {vius}_{i}}$$

[relative fitness of guide at siteⱼ in virusᵢ]) of: consensus, 0.000; MBC, 0.006; WGAN-AM, 0.090; evolutionary, 0.177; AdaLead, 0.140; CbAS, 0.134. (10b) Distributions of predicted activities for the guides designed for the variant identification objective. Top, distributions of the difference in predicted on-target and off-target activities (A(p|T₁)−A(p|T₂)) across the 100 target pairs for the baseline and generative sequence design methods. Bottom, break-down of the difference into predicted on-target activity (A(p|T₁); bottom left) and predicted off-target activity (A(p|T₂); bottom right). The mean difference in predicted on-target and off-target activities was: baseline, 0.399; WGAN-AM, 1.26; evolutionary, 1.42; AdaLead, 1.34; CbAS: 1.17.

FIG. 11a-11e—Divergence of guides from observed sequences. Histograms representing the minimum Hamming distance between the guides compared to all target sequences at a given genomic site—that is, the Hamming distance between a guide and the target sequence most similar to that guide. Shown for (11a) enterovirus B, (11b) Lassa virus segment S, (11c) influenza A virus segment 2, (11d) SARS-CoV-2, and (11e) dengue virus. The generative sequence design methods (WGAN-AM, evolutionary, AdaLead, and CbAS) tend to produce guides that are more dissimilar to any of the target sequences (that is, more artificial) than the baseline algorithms (consensus and MBC).

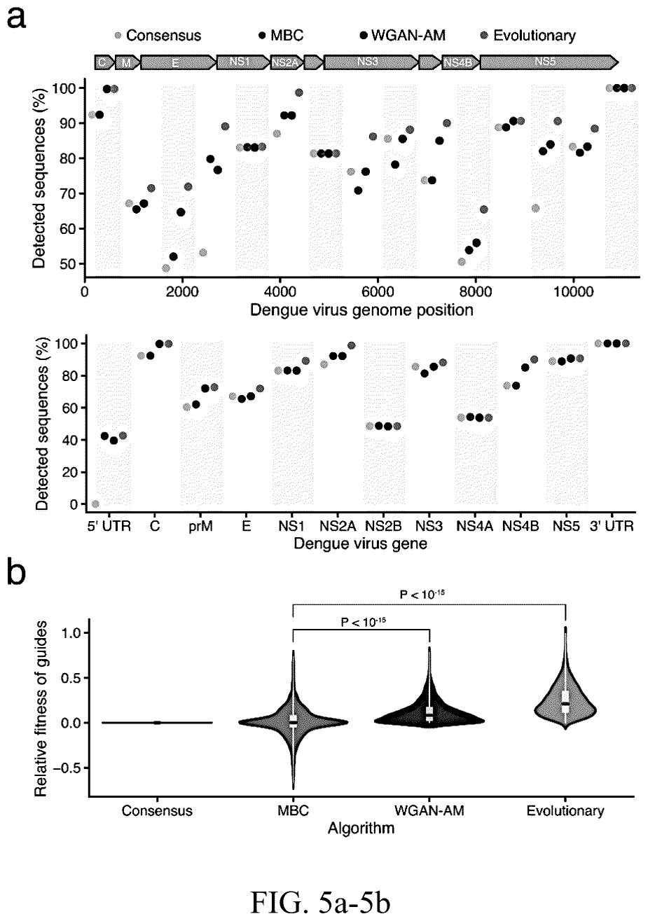

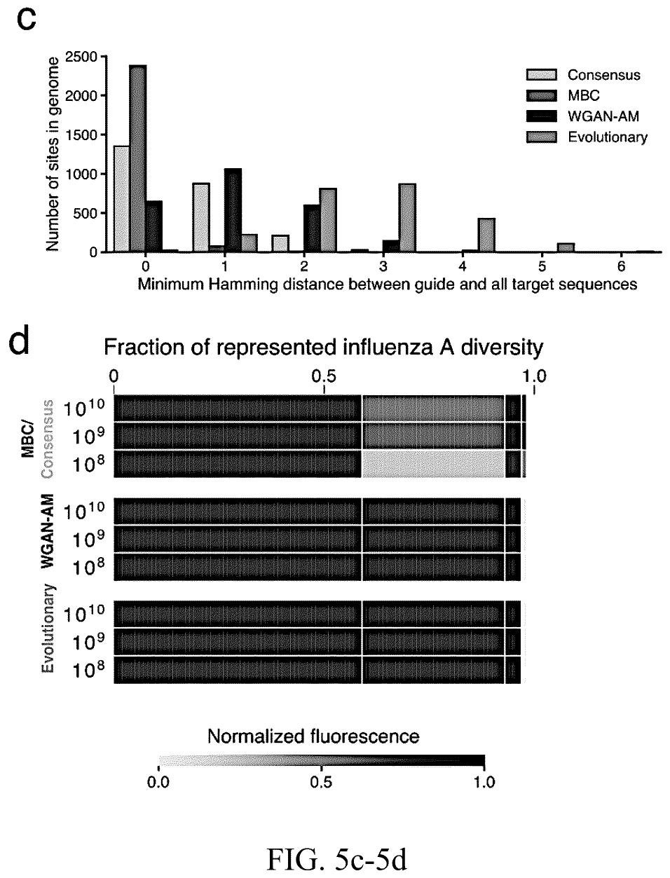

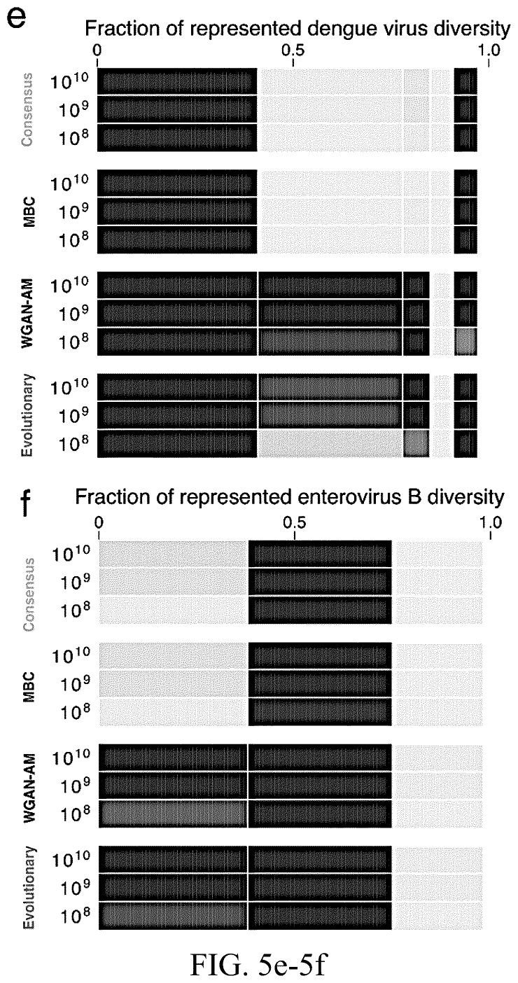

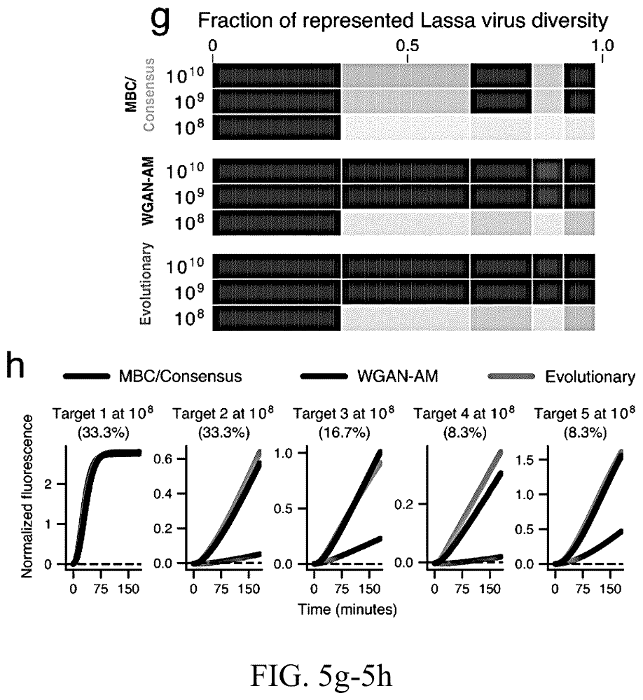

FIG. 12a-12d—Kinetic curves for multi-target detection tasks. Normalized fluorescence over the course of the reaction for the guides targeting the genomic sites whose heatmaps are shown in FIG. 5. Parentheticals indicate the percentage of all genomes represented by the target and concentrations of targets are indicated above each plot with units of copies/μL. (12a) Kinetic curves for a site in dengue virus (heatmap in FIG. 5e). (12b) Kinetic curves for a site in enterovirus B (heatmap in FIG. 5f). (12c) Kinetic curves for a site in Lassa virus segment S (heatmap in FIG. 5g). (12d) Kinetic curves for a site in influenza A virus segment 2 (heatmap in FIG. 5d).

FIG. 13a-13k—Experimental performance of multi-target detection guides. Each panel in this figure shows the experimental performance of guides designed for a specific genomic site. Panels (13a-f) represent genomic sites in the top quartile of relative performance, while panels (13g-k) represent genomic sites in the bottom quartile of relative performance (RP; see Methods for definition). The experimental data for the other four genomic sites in the top quartile of relative performance is shown in FIG. 5 and FIG. 12. The left side of each panel is a heatmap representing fluorescence at 1 hour. Each column represents a target and has width proportional to the percentage of sequence diversity it represents, while each row is a concentration of the target sequence in copies/μL. The right side of each panel is a set of curves representing the normalized fluorescence of the guides against the specified targets. The gray shading of these curves matches the gray shading of the design method used to label each heatmap. Parentheticals indicate the percentage of all genomes represented by the target. (13a) Site in dengue virus (top quartile of RP). (13b) Site in enterovirus B (top quartile of RP). (13c) Site in Lassa virus segment S (top quartile of RP). (13d) Site in influenza A virus segment 2 (top quartile of RP). (13e) Site in SARS-CoV-2 (top quartile of RP). (13f) Site in SARS-CoV-2 (top quartile of RP). (13g) Site in dengue virus (bottom quartile of RP). (13h) Site in influenza A (bottom quartile of RP). (13i) Site in enterovirus B (bottom quartile of RP). (13j) Site in SARS-CoV-2 (bottom quartile of RP). (13k) Site in Lassa virus (bottom quartile of RP).

FIG. 14—Improvement of the generative design algorithms over baseline methods when targeting variably diverse genomic sites. Relative improvement of generative algorithm-designed guides over the consensus guides grouped by the Shannon entropies of the genomic sites. The distribution is across all genomic sites in the 5 viral species considered for the multi-target detection objective (dengue virus, influenza A virus, enterovirus B, Lassa virus, and SARS-CoV-2). Relative fitness is defined as the fitness of the guide subtracted by the fitness of the consensus guide at that genomic site. By definition, if a guide has a relative fitness greater than zero, it is more fit than the consensus guide. The dots in the center of each violin represent the mean relative fitness for that decile.

FIG. 15a-15d-Experimental performance of guides designed for SNP and variant identification tasks. Normalized fluorescence over time is shown for (15a) C580Y in Pfk13, (15b) K76T in Pfcrt, (15c) Y184F in Pfmdr1, and (15d) dengue virus serotypes 1-4. The title of each plot indicates the target the guides were designed to detect as the on-target. On-target solid curves represent the fluorescence of the guide for its on-target and the dotted curves represent the fluorescence of the guide against its off-target. In (15d), there are multiple off-targets for each discrimination task, so the dotted off-target curve shows the maximum fluorescence of the guide across the off-targets computed at each time point (e.g., the off-target curves for DENV1 represent the maximal fluorescence across the DENV2, DENV3, and DENV4 targets at each time point in the reaction). All targets were present at a concentration of 10⁸ copies/μL.

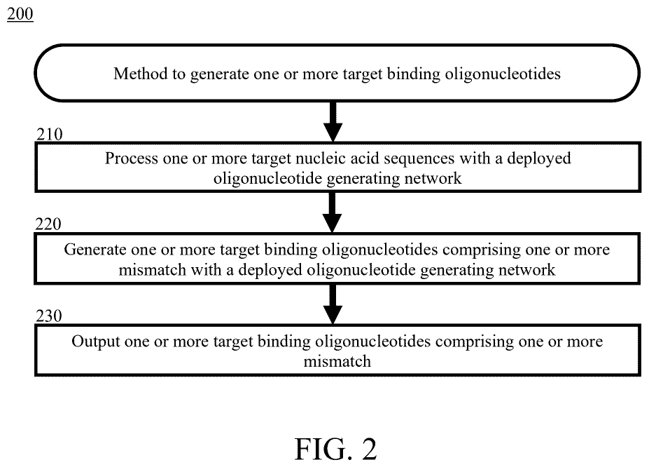

FIG. 16a-16e—Visualization of guide-target mismatches in guides designed for the multi-target detection objective. Each panel shows guide sequences and representative target sequences for one experimentally-tested genomic site in each of the five viral pathogens considered. The guides are reverse-complemented, from their sequence in a crRNA, to be in the same frame as the target sequences, and both the guides and targets are shown as DNA sequences (with T replacing U). The black blocks indicate that there is no mismatch between the guide and target, while the dark gray/near black, light gray, light gray/near white, and dark gray blocks represent A-B, G-H, T-V, and C-D mismatches respectively. The blocks in purple represent G-U wobble RNA base pairing. Parentheticals next to target names indicate the percent of sequence variation represented by that target. (16a) A site in dengue virus. The baseline guides have mismatches with target 2 at positions 10 and 11 and also have mismatches with target 3 at positions 10, 11, and 12. The algorithms in BADGERS mutate the guide at position 10 to remove a mismatch at this position with targets 2 and 3, but introduce a mismatch at this position with targets 1, 4, and 5. Experimentally, the baseline guides have nearly no fluorescence on targets 2 and 3; in contrast, the designed guides achieve strong fluorescence on targets 2 and 3, while retaining similar performance on targets 1, 4, and 5 (FIG. 5e). Thus, the designed guides can detect nearly 44% more sequence diversity. (16b) A site in enterovirus B. Here, the guides designed by the baseline approaches have three mismatches in close proximity of one another (at positions 10, 13, and 16) with target 1, the target most representative of sequence variation. The algorithms in BADGERS mutate the nucleotide at position 10 and avoid the deleterious effect of this triple mismatch, but do introduce a mismatch with target 2 at this position. The baseline guides achieve nearly no fluorescence on target 1, while the BADGERS-designed guides are highly active on target 1, and still retain robust activity on target 2 (FIG. 5f). (16c) A site in influenza A virus segment 2. The baseline guides have mismatches with target 2 at positions 6 and 10. The methods eliminate the position 6 mismatch against target 2 while not introducing mismatches to other targets (e.g., target 1) by employing G-U wobble base pairing between the RNAs. The methods implemented also mutate the nucleotide at position 10 to remove the mismatch against target 2, but by doing this, they introduce a mismatch at position 10 against target 1. These sequence changes enable the designed guides to nearly triple the fluorescence against target 2 as the baseline guides. However, because they do introduce a mismatch to target 1, they have slightly lower fluorescence at the initial timepoints against this target but still achieve saturating fluorescence by the later timepoints of the reaction (FIG. 5d). (16d) A site in Lassa virus segment S. At this site, BADGERS-designed guides achieve substantially greater fluorescence on both targets 2 and 4 (FIG. 5g). From the pattern of guide-target mismatches, it is not clear why the designed guides perform better. As one hypothesis, the close proximity of the instances of G-U wobble base pairing at positions 16 and 19 of the baseline guides might be deleterious to activity; here, the algorithms in BADGERS might be exploiting features of Cas13a targeting that are not currently well-characterized. (16e) A site in SARS-CoV-2. Previous work (FIG. 2e in ref. 1) has shown that a T-V guide-target mismatch at position 1 is strongly associated with increased guide activity. Both the WGAN-AM and evolutionary algorithms introduce this mismatch. Experimentally, the evolutionary and WGAN-AM guides both achieve slightly greater fluorescence than the baseline (MBC and consensus) guides, likely due to their position 1 mismatch (13e).

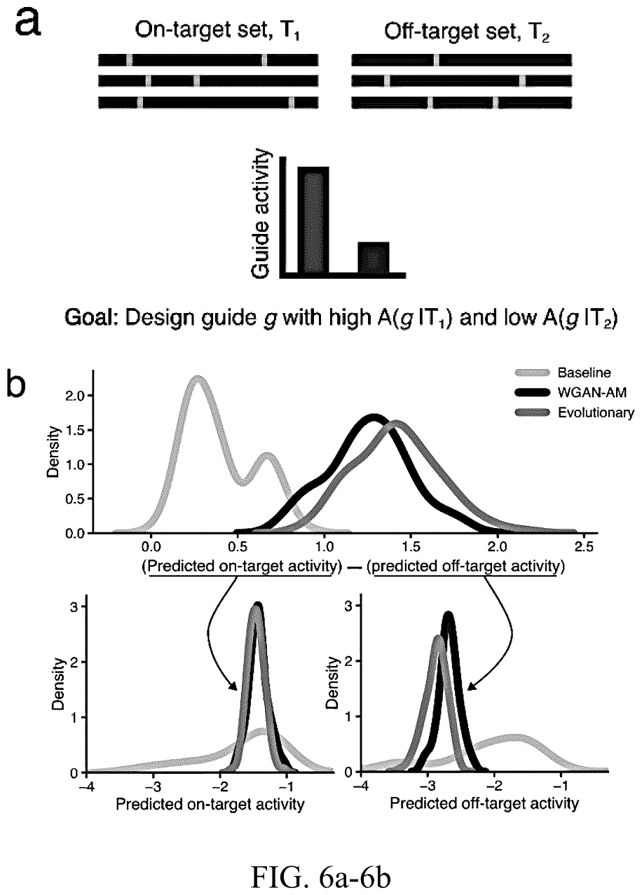

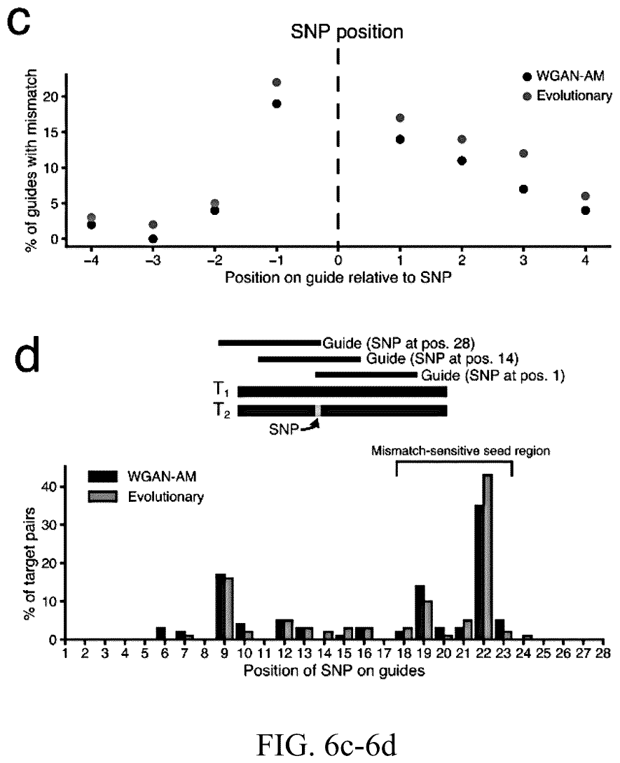

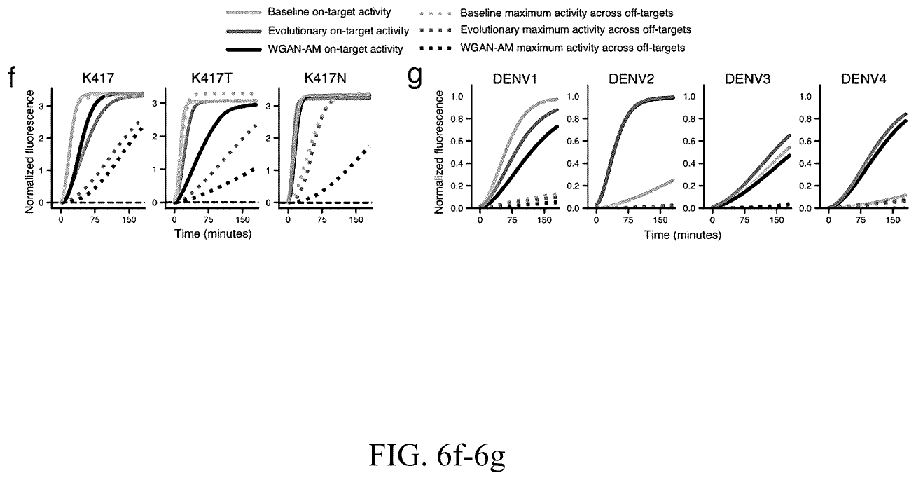

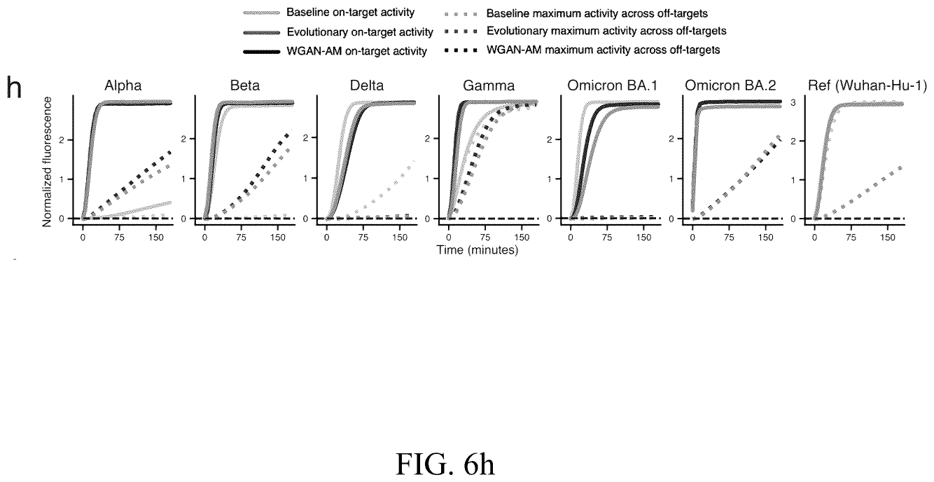

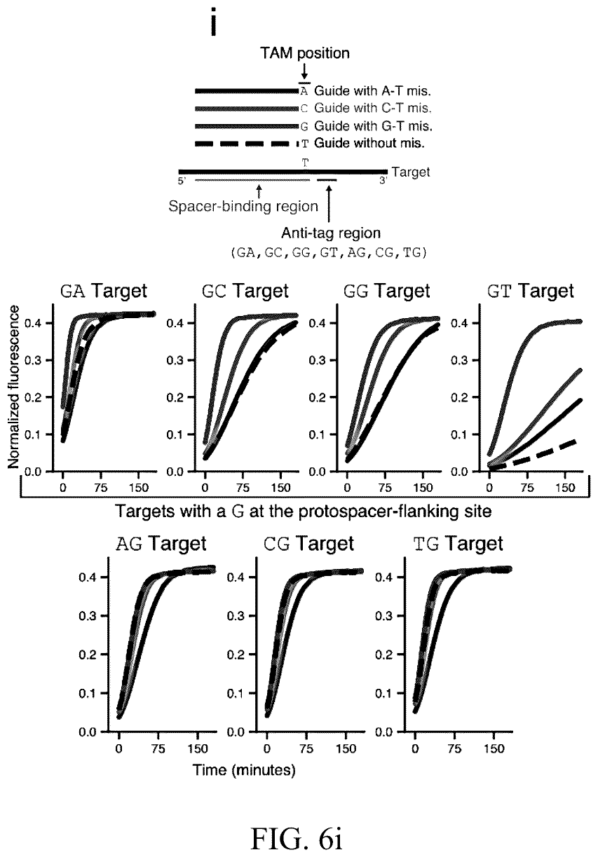

FIG. 17—In an example embodiment, the method exploits Cas13a's protospacer context preferences to design optimal variant identification guides. When targeting a SNP from a non-G nucleotide a G nucleotide, the algorithms in BADGERS often place the G nucleotide in the off-target at the PFS for optimal SNP discrimination. In this schematized example, the guides were designed to achieve high activity on the S139N target and low activity on the S139 target. Both the WGAN-AM and evolutionary algorithm positioned the guides such that the G nucleotide in the S139 off-target sequence is located at the PFS, thus lowering off-target activity, as is experimentally observed (FIG. 6e).

FIG. 18a-18d—Divergence of designed guides from the consensus sequence in an example embodiment. Fraction of designed guides that have a mismatch against the consensus genome sequence, at each position in the guide-target pairing. (18a) Fractions taken across all genomic sites from all of the five viral pathogens considered in the multi-target detection objective (dengue virus, influenza A virus, enterovirus B, Lassa virus, and SARS-CoV-2). (18b) Fractions taken across only the genomic sites with a G nucleotide at the PFS, again across all of the five viral pathogens considered in the multi-target detection objective. On this subset of sites, the designed guides are relatively likely to have a terminal mismatch in the protospacer, suggesting the benefit of such a mismatch when there is a G nucleotide at the PFS. (18c) Fractions taken across all genomic sites considered in the variant identification detection objective. (18d) Fractions taken across only the genomic sites with a G nucleotide at the PFS amongst the sites considered in the variant identification objective. In (18c) and (18d), T₁ is the on-target sequence.

FIG. 19a-19c—Evaluation of the ability of the tag-adjacent mismatch to rescue guide activity for different target sets. Normalized fluorescence curves are shown for the target sets where the tag-adjacent position (TAM position) in the target is a (19a) G nucleotide, (19b) A nucleotide, or (19c) C nucleotide. All guides are identical to the targets at positions 1-27 in the protospacer. The gray lines represent guides with a tag-adjacent mismatch (at position 28), while the black dashed lines represent guides without a mismatch to the target. The dinucleotide above each plot indicates the first two nucleotides of that target's anti-tag region, and the guide sequences represented in the schematic are reverse-complemented compared to the spacer sequence in the CRISPR RNA. The same curves for the target set with a T nucleotide at the tag-adjacent position are shown in FIG. 6i.

FIG. 20a-20b—Visualization of the fitness function for the variant identification objective. Gray shading represent the value of the variant identification fitness function according to on-target (T1) and off-target (T2) activities (see Methods of Example 1 for the function). A maximally-fit guide g has a low maximum activity across the off-target set T2, max_{t∈T2}A(g|t), and a high minimum activity across the on-target set T1, max_{t∈T2}A(g|t). (20a) Using hyperparameters determined by the WGAN-AM random search. (20b) Using hyperparameters determined by the evolutionary algorithm random search.

FIG. 21a-21b—Properties of WGAN-AM algorithm. (21a) Architecture of generative model of Wasserstein generative adversarial network (WGAN) for generating guides. The input into the generative model, the latent vector z, is a 10-dimensional vector sampled from the standard normal distribution. z is up-sampled through a linear layer and reshaped and padded to create a 48 by 10 matrix. In the concat operation, the one-hot-encoded consensus sequence of the target set T (with dimensions 48 by 4) is concatenated with the output of the previous layer to create a 48 by 14 matrix. This matrix is passed through three residual blocks. Each residual block (resblock) consists of two layers, with each layer containing a 1D convolutional layer with 14 filters of stride 1 and width 3. Next, in the conv operation, a convolutional layer with 4 filters of width 1 and stride 1 is applied to the output of the resblock to create 4-channel encoded guide sequence. Finally, the matrix is cropped to remove the 10 by 4 context on each side of the guide protospacer sequence, and the resulting 28 by 4 matrix is passed through a softmax layer so that each element in the matrix represents the probability of having a certain base at that position. The guide sequence contains the base at each position with the greatest probability. (21b) Relationship between the Euclidean norm of the latent variable input to the WGAN's generator network and the number of mutations introduced into the guide relative to the consensus sequence. All guides with a Hamming distance ≤10 are shown, and the computational experiment was performed as described herein.

FIG. 22—A block flow diagram depicting methods to generate one or more target binding oligonucleotides, in accordance with certain examples of the technology disclosed herein.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a,” “an,” and “the” include both singular and plural referents unless the context dictates otherwise.

The term “optional” or “optionally” means that the subsequently described event, circumstance, or substituent may or may not occur. The description includes instances where the event or circumstance occurs and instances where it does not.

The recitation of numerical ranges by endpoints includes all numbers and fractions subsumed within the respective ranges and the recited endpoints.

The terms “about” or “approximately,” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is also specifically and preferably disclosed.

As used herein, a “biological sample” may contain whole cells and/or live cells and/or cell debris. The biological sample may contain (or be derived from) a “bodily fluid.” The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammal organism, for example, by puncture or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells, and the progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment,” “an embodiment,” and “an example embodiment” means that a particular feature, structure, or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures, or characteristics may be combined in any suitable manner, as would be apparent to a person skilled in the art from this disclosure in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each publication, published patent document, or patent application was specifically and individually indicated as being incorporated by reference.

### Overview

Molecular diagnostic technologies have transformed the detection and surveillance of infectious pathogens. The performance of these assays depends critically on the diagnostic oligonucleotide sequences they employ to bind to targeted nucleic acids. Current techniques for designing such diagnostic oligonucleotides, including PCR primers and CRISPR guide RNAs, select sequences directly from those in nature or from a simple function of natural sequences, e.g., a consensus, according to machine-learned discriminative models or thermodynamic heuristics¹⁻⁶. Thus, outputs of these techniques are identical (or nearly identical) to the input, natural sequences. However, natural diagnostic guide sequences may not perform the best in every application. Artificial diagnostic sequences—ones that differ from any natural sequence by one or more mismatches—may achieve superior diagnostic activity than their natural counterparts. While typical artificial diagnostic sequences are task dependent and therefore limited in capability, the methods described herein provide for multipurpose artificial diagnostic sequences with improved selectivity. These methods advance the design capabilities for artificial diagnostic sequences because they can cover the enormous search space, which includes the entire target nucleic acid sequence; potential off-target sequences; as well as variations for each individual nucleic acid. These methods further advance the design capabilities by effectively inserting mismatches that enhance the functionality of the artificial diagnostic sequence. This solution of improving the performance through mismatch insertion is not only counterintuitive by typical design approaches but is also a unique approach to diversifying the limited capabilities of traditionally designed artificial diagnostic sequences.

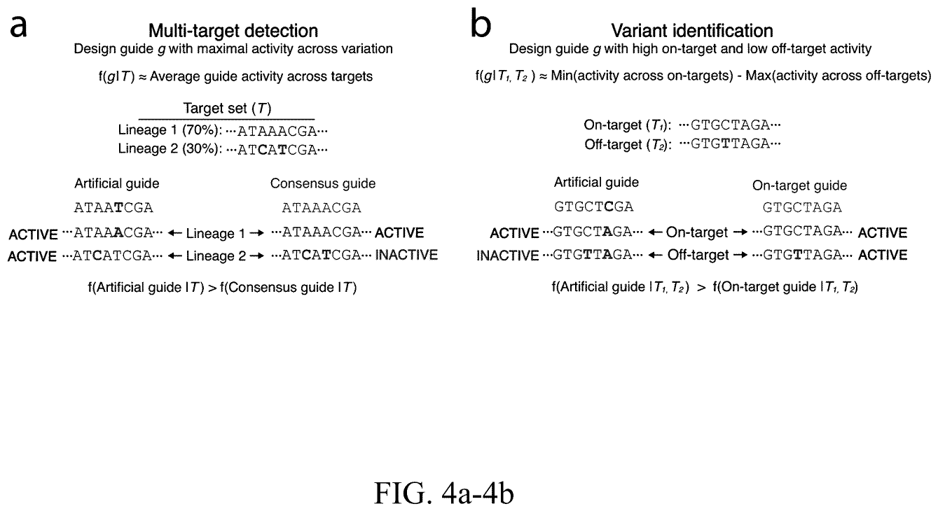

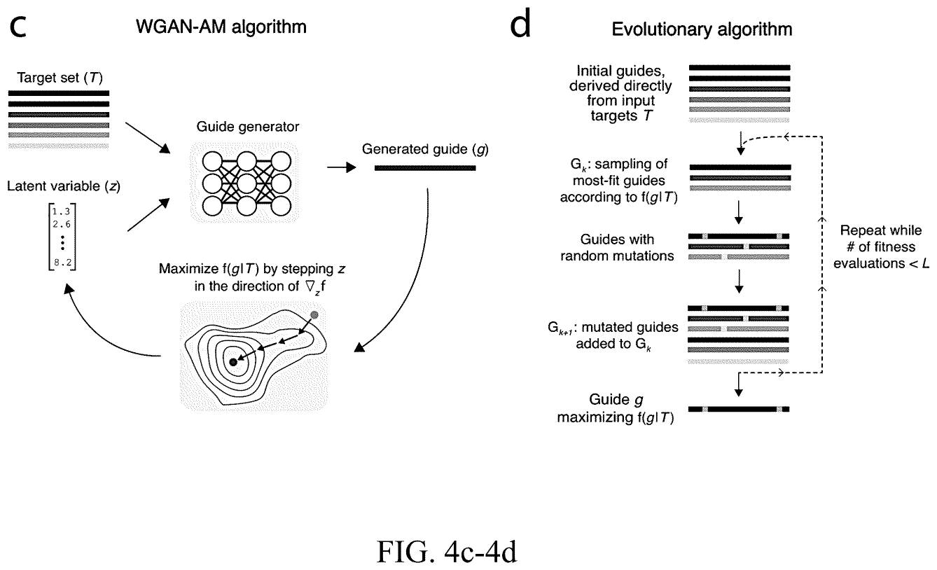

The methods described herein utilize a generative design approach to construct artificial diagnostic oligonucleotide sequences to increase performance on key detection tasks. For instance, if the goal were to detect all forms of a genetically diverse pathogen (“multi-target detection”), and there are several polymorphisms in an otherwise efficacious target region, an artificial oligonucleotide sequence could more sensitively detect all combinations of polymorphisms than any sequence that fully matches one particular combination (FIG. 4a). This would enable improved diagnostic sensitivity. Alternatively, if the goal were to distinguish between two highly similar targets (“variant identification”), even those that differ by a single nucleotide polymorphism (SNP), an artificial sequence with optimally-positioned mismatches could achieve greater specificity than a sequence identical to one target (FIG. 4b). This would enable more accurate identification of mutation(s) in a pathogen or discriminate between pathogen lineages. Previous work^{16, 17} has described heuristic rules to introduce handcrafted specificity-enhancing mismatches into diagnostic oligonucleotide sequences, but these strategies are custom-made and are limited by the degree of variation they can distinguish.

For example, generating maximally-fit biological sequences can transform diagnostic oligonucleotide design. Oligonucleotide generating algorithms for designing maximally fit, artificial diagnostic oligonucleotides—with multiple mismatches to any natural sequence—can be tailored for desired properties around nucleic acid diagnostics. These guides offer more sensitive detection of diverse pathogens and discrimination of pathogen variants compared to guides derived directly from natural sequences, and illuminate interpretable design principles that enhance overall diagnostic assay design.

The embodiments disclosed herein can utilize machine learning to generating target binding oligonucleotides, as further defined below, which in turn allows for targeting one or more nucleic acid sequences, in some instances comprising one or more polymorphism.

In one aspect, technologies herein provide methods to generate one or more target binding oligonucleotides, comprising: processing one or more target nucleic acid sequences with a deployed oligonucleotide generating network and generating, by the deployed oligonucleotide generating network, one or more engineered target binding oligonucleotides, wherein the one or more engineered target binding oligonucleotides comprise one or more mismatches.

### Target Nucleic Acid Sequences

The target nucleic acid sequence the target binding oligonucleotides are designed to bind may be any nucleic acid sequence. In an example embodiment, the target nucleic acid sequence comprises polymorphisms. In an example embodiment, the target nucleic acid comprises a single nucleotide polymorphism. In an example embodiment, the target nucleic acid comprises multiple polymorphisms. The polymorphisms may be consecutive (e.g., continuous sequence of polymorphisms), disjointed (e.g., polymorphism separated by non-polymorphic nucleotides), or a combination thereof.

In an embodiment, a target nucleic acid sequence can be any sequence for which a diagnostic is desired. In an embodiment, the target nucleic acid sequence can be a DNA or RNA-based target sequence. In an embodiment, the target nucleic acid sequence can be a target sequence that identifies a disease or cell state. In an embodiment, the target nucleic acid sequence can be a target nucleic acid sequence that identifies a pathogen, such as a virus, bacteria, fungus, or protozoa.

In an example embodiment, the target nucleic acid sequence is a synthetic sequence such as a peptide nucleic acid (PNA), locked nucleic acid (LNA) (also known as bridged nucleic acid (BNA)). PNAs are engineered DNA comprising a peptide polymer or pseudo-peptide polymer backbone instead of the deoxyribose phosphate backbone. For example, the PNA backbone may comprise of repeating N-(2-aminoethyl)-glycine units linked by peptide bonds. LNAs generally comprise phosphodiester backbones and 2′-4′ methylene bridges connecting monomer nucleotides. See e.g., Pellestor, F., Paulasova, P. The peptide nucleic acids (PNAs) are powerful tools for molecular genetics and cytogenetics. Eur J Hum Genet 12, 694-700 (2004).

In the context of the formation of a CRISPR complex, such as a complex formed by a programmable nucleic acid modifying agent composition of the present invention, target nucleic acid sequence refers to a sequence in a polynucleotide to which a guide sequence is designed to have complementarity, where hybridization between a target nucleic acid sequence and a guide sequence promotes the formation of a CRISPR complex. A target nucleic acid sequence may comprise RNA polynucleotides. The term “target RNA” refers to an RNA polynucleotide being or comprising the target nucleic acid sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In an embodiment, a target nucleic acid sequence is located in the nucleus or cytoplasm of a cell. Other forms of the target polynucleotide are described elsewhere herein.

### Example System Architectures

Turning now to the drawings, in which numerals represent like (but not necessarily identical) elements throughout the figures, example embodiments are described in detail.

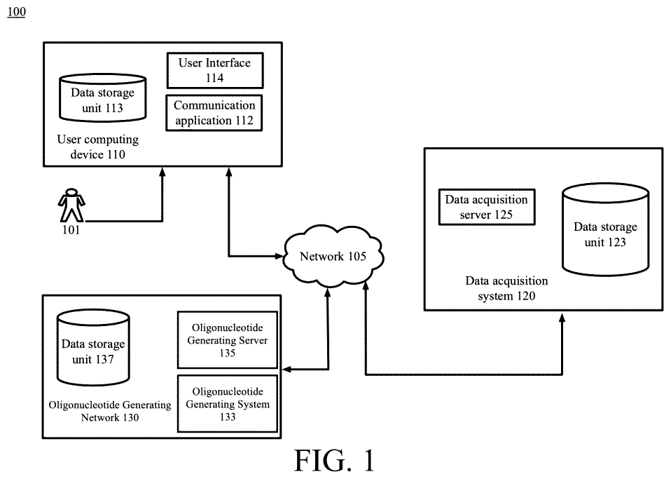

FIG. 1 is a block diagram depicting a system 100 that generates one or more engineered target-binding oligonucleotides and performs machine learning on one or more target nucleic acid sequences. In an embodiment, a user 101 associated with a user computing device 110 must install an application or make a feature selection to obtain the benefits of the techniques described herein.

As depicted in FIG. 1, system 100 includes network computing devices/systems 110, 120, and 130 that are configured to communicate with one another via one or more networks 105 or via any suitable communication technology.

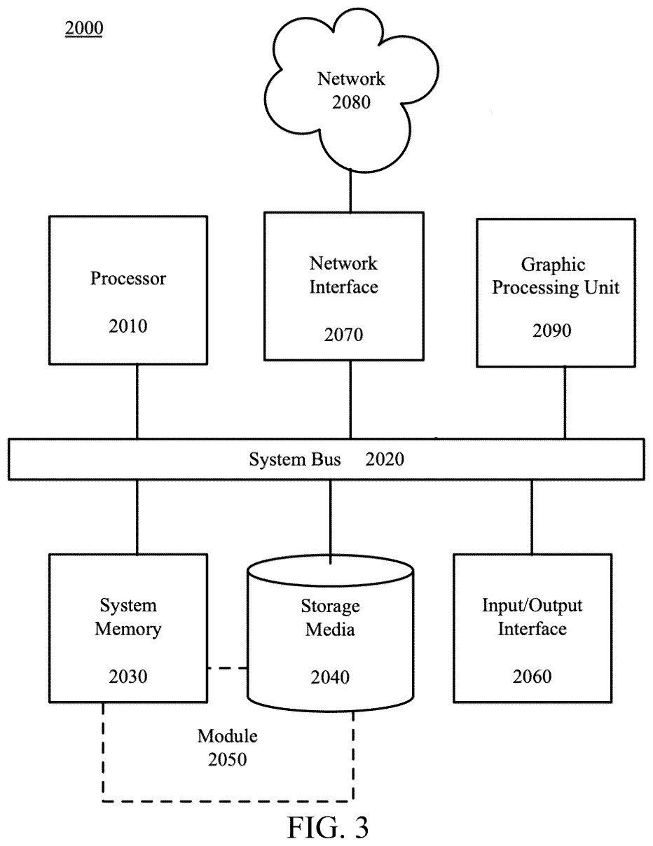

Each network 105 includes a wired or wireless telecommunication means by which network devices/systems (including devices 110, 120, and 130) can exchange data. For example, each network 105 can include any of those described herein such as the network 2080 described in FIG. 3 or any combination thereof or any other appropriate architecture or system that facilitates the communication of signals and data. Throughout the discussion of example embodiments, it should be understood that the terms “data” and “information” are used interchangeably herein to refer to text, images, audio, video, or any other form of information that can exist in a computer-based environment. The communication technology utilized by the devices/systems 110, 120, and 130 may be similar networks to network 105 or an alternative communication technology.

Each network computing device/system 110, 120, and 130 includes a computing device having a communication module capable of transmitting and receiving data over the network 105 or a similar network. For example, each network device/system 110, 120, and 130 can include any computing machine 2000 described herein and found in FIG. 3 or any other wired or wireless, processor-driven device. In the example embodiment depicted in FIG. 1, the network devices/systems 110, 120, and 130 are operated by user 101, data acquisition system operators, and oligonucleotide generating operators, respectively.

The user computing device 110 includes a user interface 114. The user interface 114 may be used to display a graphical user interface and other information to the user 101 to allow the user 101 to interact with the data acquisition system 120, the oligonucleotide generating network 130, and others. The user interface 114 receives user input for data acquisition and/or machine learning and displays results to user 101. In another example embodiment, the user interface 114 may be provided with a graphical user interface by the data acquisition system 120 and or the oligonucleotide generating network 130. The user interface 114 may be accessed by the processor of the user computing device 110. The user interface may display 114 may display a webpage associated with the data acquisition system 120 and/or the oligonucleotide generating network 130. The user interface 114 may be used to provide input, configuration data, and other display directions by the webpage of the data acquisition system 120 and/or the oligonucleotide generating network 130. In another example embodiment, the user interface 114 may be managed by the data acquisition system 120, the oligonucleotide generating network 130, or others. In another example embodiment, the user interface 114 may be managed by the user computing device 110 and be prepared and displayed to the user 101 based on the operations of the user computing device 110.

The user 101 can use the communication application 112 on the user computing device 110, which may be, for example, a web browser application or a stand-alone application, to view, download, upload, or otherwise access documents or web pages through the user interface 114 via the network 105. The user computing device 110 can interact with the web servers or other computing devices connected to the network, including the data acquisition server 125 of the data acquisition system 120 and the oligonucleotide generating server 135 of the oligonucleotide generating network 130. In another example embodiment, the user computing device 110 communicates with devices in the data acquisition system 120 and/or the oligonucleotide generating network 130 via any other suitable technology, including the example computing system described below.

The user computing device 110 also includes a data storage unit 113 accessible by the user interface 114, the communication application 112, or other applications. The example data storage unit 113 can include one or more tangible computer-readable storage devices. The data storage unit 113 can be stored on the user computing device 110 or can be logically coupled to the user computing device 110. For example, the data storage unit 113 can include on-board flash memory and/or one or more removable memory accounts or removable flash memory. In another example embodiment, the data storage unit 113 may reside in a cloud-based computing system.

An example data acquisition system 120 comprises a data storage unit 123 and an acquisition server 125. The data storage unit 123 can include any local or remote data storage structure accessible to the data acquisition system 120 suitable for storing information. The data storage unit 123 can include one or more tangible computer-readable storage devices, or the data storage unit 123 may be a separate system, such as a different physical or virtual machine or a cloud-based storage service.

In an embodiment, the data acquisition server 125 communicates with the user computing device 110 and/or the oligonucleotide generating network 130 to transmit requested data. The data may include one or more target nucleic acid sequences.

An example oligonucleotide generating network 130 comprises an oligonucleotide generating system 133, an oligonucleotide generating server 135, and a data storage unit 137. The oligonucleotide generating server 135 communicates with the user computing device 110 and/or the data acquisition system 120 to request and receive data. The data may comprise the data types previously described in reference to the data acquisition server 125.

The oligonucleotide generating system 133 receives an input of data from the oligonucleotide generating server 135. In an embodiment, the input data is a target binding oligonucleotide. In an embodiment, the target binding oligonucleotide is a guide nucleic acid sequence, small interfering RNA (siRNA), microRNA (miRNA), diagnostic primer nucleic acid sequence, probe nucleic acid sequence, peptide nucleic acid (PNA), or locked nucleic acid (LNA). The oligonucleotide generating system 133 can comprise one or more functions to implement any of the mentioned training methods to learn and then generate a target binding oligonucleotide from a target nucleic acid sequence. Any suitable architecture may be applied to learn a target-binding oligonucleotide from a target nucleic acid sequence. In an embodiment, the objective network may also be referred to as an objective function.

The data storage unit 137 can include any local or remote data storage structure accessible to the oligonucleotide generating network 130 suitable for storing information. The data storage unit 137 can include one or more tangible computer-readable storage devices, or the data storage unit 137 may be a separate system, such as a different physical or virtual machine or a cloud-based storage service.

In an alternate embodiment, the functions of either or both of the data acquisition system 120 and the oligonucleotide generating network 130 may be performed by the user computing device 110.

It will be appreciated that the network connections shown are examples, and other means of establishing a communications link between the computers and devices can be used. Moreover, those having ordinary skill in the art having the benefit of the present disclosure will appreciate that the user computing device 110, data acquisition system 120, and the oligonucleotide generating network 130 illustrated in FIG. 1 can have any of several other suitable computer system configurations. For example, a user computing device 110 embodied as a mobile phone or handheld computer may not include all the components described above.

In an embodiment, the network computing devices and any other computing machines associated with the technology presented herein may be any type of computing machine such as, but not limited to, those discussed in more detail with respect to FIG. 3. Furthermore, any modules associated with any of these computing machines, such as modules described herein, or any other modules (scripts, web content, software, firmware, or hardware) associated with the technology presented herein may by any of the modules discussed in more detail with respect to FIG. 3. The computing machines discussed herein may communicate with one another as well as other computer machines or communication systems over one or more networks, such as network 105. The network 105 may include any type of data or communications network, including any of the network technology discussed with respect to FIG. 3.

### EXAMPLE PROCESSES

The example methods illustrated in FIG. 2 are described hereinafter with respect to the components of the example architecture 100. The example methods can also be performed with other systems and in other architectures, including similar elements.

Referring to FIG. 2, and continuing to refer to FIG. 1 for context, a block flow diagram illustrates method 200 to generate one or more target-binding oligonucleotides in accordance with certain examples of the technology disclosed herein.

### Processing of Target Nucleic Acid Sequences

In block 210, the oligonucleotide generating system 133 receives and/or processes data comprising one or more target nucleic acid sequences. In an embodiment, the data includes more than one target nucleic acid sequence. The multiple target nucleic acid sequences can be a whole genome, any size fragment thereof, or a combination thereof. The target nucleic acid sequences can be from any number of sequences necessary to represent a genus, any number of sequences to represent a particular species or a combination thereof. The target nucleic acid sequences can be from a prokaryote, eukaryote, or a combination thereof. In an example embodiment, the target nucleic acid can be from a mammal. In an example embodiment, the target nucleic acid sequence can be from a pathogen, such as a virus, bacteria, fungi, or protozoa. In an example embodiment, the target nucleic acid sequences can be from a diseased cell, such as a cancer cell. For example, the oligonucleotide generating system 133 can process the target nucleic acid sequences for variant identification, which can include high on-target specificity and/or low off-target activity, or multi-target detection, which can include high activity across variations of the target nucleic acid sequences.

In an embodiment, the sequence data (e.g., string data) is transformed into numerical data (e.g., integer or float data. In an embodiment, the one or more target nucleic acid sequences (e.g., string, integer, or float data) are transformed into a vector or matrix. The transformed one or more target nucleic acid sequences can then be operated on. In an embodiment, the matrix or vector is reshaped into a latent vector or latent matrix or an embedded vector or embedded matrix. A latent or embedded vector or matrix, further referred to as a latent matrix, expands the input space from the one or more target nucleic acid sequences to one or more target nucleic acid sequences with additional variables corresponding to the input. These additional variables can correspond to numerical representations of characteristic features of the input data. In an embodiment, the vector or matrix space of the additional variables can have higher, lower, or the same dimensionality as the one or more target nucleic acid sequences. In an embodiment, the vector or matrix space of the additional variables has lower dimensionality than the one or more target nucleic acid sequences. In an embodiment, the latent matrix is padded on one or more sides. Padding, in general, refers to increasing the size of the dimensions of a vector or matrix by adding, for example, additional columns or rows to a matrix or vector. In an embodiment, the latent matrix is zero-padded such that zeros are added to one or more sides of the latent matrix.

In an embodiment, the transformed matrix or latent matrix is concatenated with a conditional matrix. The conditional matrix further defines the input and can direct the machine learning model on how to operate on the data. For example, given a target nucleic acid sequence or set of target nucleic acid sequences, the conditional matrix can define characteristics of the one or more target binding oligonucleotides, e.g., size of the target binding oligonucleotides and or composition of the target binding oligonucleotides such as the percentage and/or grouping of the same nucleic acid type. The conditional matrix can undergo the same processing as described above in the paragraph as above.

The features of the input (e.g., length of the one or more target nucleic acid sequences) directly affect the complexity of generating one or more target binding oligonucleotides. By way of an example, an input of one target nucleic acid sequence is processed by the oligonucleotide generating network to generate one target binding oligonucleotide. For every 1:1 sequence to sequence alignment, the search space scales at a factor of 4ⁿ wherein n equals the number of nucleic acids in the one or more target binding oligonucleotides. This alone necessarily results in a rapidly expanding search space for the machine learning model. For example, for every 5 nucleic acids added to a target binding oligonucleotide the search space increases by a thousandfold (1×10³) (e.g., 4⁵ equals ˜1×10³; 4¹⁰ equals ˜1×10⁶; 4¹⁵ equals ˜1×10⁹; 4²⁰ equals ˜1×10¹²). Therefore, to generate a target binding oligonucleotide with one or more mismatches of twenty (20) nucleic acids in length, the oligonucleotide generating network has to consider a space of 1 trillion possibilities. As a further example, a single target nucleic acid sequence input can be larger in the length than a single target binding oligonucleotide output. The oligonucleotide generating network would then need to search, at least, double the search space for every single shift of the single target binding oligonucleotide along the length of the target nucleic acid sequence as the one or more mismatches can change efficacy per position in a given target binding oligonucleotide (essentially a shift of the nucleic acid sequence would equate to sampling another target nucleic acid sequence).

In an embodiment, processing includes processing the one or more target nucleic acid sequences with an objective function. In an embodiment, the objection function modifies the sequence of the one or more target nucleic acid sequences, predicts one or more desired features of the sequence, and selects only the modified one or more target nucleic acid sequences it predicts have improved desired features (etc., binding strength, on-target specificity, off-target binding, reduced complementariness, etc.). For example, the objective function can mutate one or more nucleic acids in the one or more target nucleic acid sequences. If it predicts the sequence has improved desired features, then the mutated one or more target nucleic acid sequences can be passed forward (e.g., to another process such as activation maximization, evolutionary network, fitness evaluation, etc.). In an embodiment, the objection function is performed iteratively and the mutated one or more target nucleic acid sequences are only passed forward once the objection function can no longer predict an improved sequence.

Any of the processes noted above can be included and combined in the oligonucleotide generating network.

### Generating Target Binding Oligonucleotides

In block 220, a machine learning module of the oligonucleotide generating system 133 receives target nucleic acid sequence data processed in block 210 to identify and search over the landscape of possible target binding oligonucleotides to generate maximally fit targeting binding oligonucleotides for the received target nucleic acid sequences. The processing of the one or more target nucleic acid sequences is performed by the machine learning algorithm based on data collected by the data acquisition system 120 and/or data within the data storage unit 137. Accordingly, human analysis or cataloging is not required. The process is performed automatically by the oligonucleotide generating network 130 without human intervention. The amount of data typically collected includes thousands to tens of thousands of data items for the target nucleic acid sequences. The total number of users may include all users accessing the system or a portion of users using a particular aspect of the system (e.g., the portion of users using the mobile application as opposed to those using a web browser portal). Human intervention in the process is not useful or required because the amount of data is too great. A team of humans would not be able to catalog or analyze the data in any useful manner. Moreover, a human cannot access target nucleic acid sequences and from that raw data predict one or more engineered target binding oligonucleotides that will successfully bind to the target nucleic acid sequence.

### Mismatches

In an embodiment, the methods disclosed herein are configured to generate target binding oligonucleotides comprising one or more mismatches relative to the target nucleic acid sequence to which it is designed to bind. A mismatch generally refers to a nucleotide that is not the natural complement of a nucleotide in the target nucleic acid sequence (e.g., C-T, C-A, T-G, G-A, C-C, T-T, A-A, G-G, etc.). This feature makes the generated target binding oligonucleotide an artificial (i.e., non-naturally occurring) sequence relative to the target nucleic acid sequence. These mismatches are generated by the methods disclosed herein to maintain effective targeting of a nucleic acid sequence or a range of target nucleic acid sequences (e.g. ability to detect different combinations of polymorphisms or distinguish between highly similar targets (variant identification).

In an embodiment, a mismatch can allow a target binding oligonucleotide to distinguish two or more targets that vary by a single nucleotide, such as a single nucleotide polymorphism (SNP), variation, or (point) mutation. Thus, for two targets, or a set of targets, a target binding oligonucleotide may be generated with a nucleotide sequence that is complementary to one of the targets i.e., the on-target SNP. The target binding oligonucleotide can be further generated to have a synthetic mismatch. As used herein a “synthetic mismatch” refers to an artificial (e.g., non-naturally occurring) mismatch that is introduced upstream or downstream of the naturally occurring SNP, such as at most 5 nucleotides upstream or downstream, for instance 4, 3, 2, or 1 nucleotide upstream or downstream, preferably at most 3 nucleotides upstream or downstream, more preferably at most 2 nucleotides upstream or downstream, most preferably 1 nucleotide upstream or downstream (i.e., adjacent the SNP). Thus, the systems disclosed herein can be designed to distinguish SNPs within a population. For, example the systems can be used to distinguish pathogenic strains that differ by a single SNP or detect certain disease specific SNPs, such as but not limited to, disease associated SNPs, such as without limitation cancer associated SNPs.

In an example embodiment, wherein the mismatch is a nucleotide not complementary to a corresponding nucleotide of at least 1 mismatch, at least 2 mismatches, at least 3 mismatches, at least 4 mismatches, at least 5 mismatches, at least 6 mismatches, at least 7 mismatches, at least 8 mismatches, at least 9 mismatches, at least 10 mismatches, at least 11 mismatches, at least 12 mismatches, at least 13 mismatches, at least 14 mismatches, at least 15 mismatches, at least 16 mismatches, at least 17 mismatches, at least 18 mismatches, at least 19 mismatches, at least 20 mismatches, at least 21 mismatches, at least 22 mismatches, at least 23 mismatches, at least 24 mismatches, or at least 25 mismatches. In an example embodiment, the mismatch is a nucleotide not complementary to a corresponding nucleotide of 1 mismatch, 2 mismatches, 3 mismatches, 4 mismatches, 5 mismatches, 6 mismatches, 7 mismatches, 8 mismatches, 9 mismatches, 10 mismatches, 11 mismatches, 12 mismatches, 13 mismatches, 14 mismatches, 15 mismatches, 16 mismatches, 17 mismatches, 18 mismatches, 19 mismatches, 20 mismatches, 21 mismatches, 22 mismatches, 23 mismatches, 24 mismatches, or 25 mismatches. In an example embodiment, wherein two or more mismatches are within a 60-, within a 50-, within a 40-, within a 30-, within a 20-, within a 10-, within a 5-nucleotide range. In an example embodiment, wherein two or more mismatches are within a 5-, within a 10-, within a 15-, within a 20-, within a 25-, within a 30-, within a 35-, within a 40-, within a 45-, within a 50-, within a 55-, within a 60-, within a 65-, within a 70-, within a 75-, within a 80-, within a 85-, within a 90-, within a 95-, or within a 100-nucleotide range.

Accordingly, the machine learning module may be used to design target binding oligonucleotides that optimize an objective function across a target nucleic acid sets sequence variation. This may be used to design target binding oligonucleotides that can detect multiple targets that may vary in nucleotide sequence. In an embodiment, the machine learning module may optimize an objective function across a genome's sequence variation. An example objective function is:

$${f}_{M} ( g \vert T ) = {E}_{t ∈ T} [ A ( g \vert T ) ] = {\sum }_{t ∈ T} {w}_{t} \cdot A ( g \vert T )$$

where T is the set of genome target nucleic acid sequences and t∈T is a single target nucleic acid. Genome sequence diversity varies spatially and temporally, so the wₜ represents a prior probability of encountering a target t∈T. A uniform prior distribution over all the target nucleic acid sequences may be set but also may be modified as certain target nucleic acid sequences become more prevalent or less prevalent in a geographic region of interest or over time.

In another embodiment, the machine learning module may be configured to design target binding oligonucleotides that optimally differentiate between closely related sequences, maximizing activity against one set of target nucleic acid sequences (on-target) while minimizing activity against a separate but homologous set of non-target nucleic acid sequences (off-target). This approach may be used to design targeting binding oligonucleotides for identification of variants. An example objection function for that purpose is:

$${f}_{D} ( g \vert {T}_{1} , {T}_{2} ) = ( \frac{1}{1 + {ae}^{k ( - logsumexp ( - A ( g \vert {T}_{1} ) ) - o )}} - 1 ) + r \cdot ( \frac{- 1}{1 + {ae}^{k ( logsumexp ( A ( g \vert {T}_{2} ) ) - o )}} )$$

where T₁ represents on target nucleic acid sequences, T₂ represents off target nucleic acid sequences and a, k, and r are all parameters that modulate the slope and curvature of the objective function. The values of these parameters were determined through a random search procedure described in the hyperparameter search section of the Methods. We use log sumexp(A(g|Tᵢ)) as shorthand for log sumexp({A(g|t)∀t∈Ti}), where log sumexp(x₁, . . . , xₙ)=log (exp(x₁)+ . . . +exp(xₙ)) is a smooth approximation to the maximum. The value of f_{D}(g|T₁,T₂) increases as the minimum activity of the guide across the on-target set increases and the maximum activity of the guide across the off-target set decreases.

In an embodiment, the machine learning algorithm is a conditional generative adversarial network with activation maximization (WGAN-AM) which is described in further detail in the Working Examples section below. In another embodiment, the machine learning algorithm that used is an evolutionary algorithm, which is also described in further detail in the Working Example section below.

Using the guidance provided herein, alternate machine algorithms may be employed by the machine learning module to design targeting binding oligonucleotides. In an example embodiment, the machine learning module can be trained using techniques such as unsupervised, supervised, semi-supervised, reinforcement learning, transfer learning, incremental learning, curriculum learning techniques, and/or learning to learn. Training typically occurs after selection and development of a machine learning module and before the machine learning module is operably in use. In an embodiment, the training data used to teach the machine learning module can comprise input data such as one or more target nucleic acid sequences and the respective target output data such as one or more target binding oligonucleotides. In an embodiment, the training data used to teach the machine learning module can comprise input data such as one or more target binding oligonucleotides and the one or more target nucleic acid sequences and the respective target output data such as an activity score.

### Unsupervised and Supervised Learning

In an example embodiment, unsupervised learning is implemented. Unsupervised learning can involve providing all or a portion of unlabeled training data to a machine learning module. The machine learning module can then determine one or more outputs implicitly based on the provided unlabeled training data. In an example embodiment, supervised learning is implemented. Supervised learning can involve providing all or a portion of labeled training data to a machine learning module, with the machine learning module determining one or more outputs based on the provided labeled training data, and the outputs are either accepted or corrected depending on the agreement to the actual outcome of the training data. In an embodiment, supervised learning of machine learning system(s) can be governed by a set of rules and/or a set of labels for the training input, and the set of rules and/or set of labels may be used to correct inferences of a machine learning module.

### Semi-Supervised and Reinforcement Learning

In an embodiment, semi-supervised learning is implemented. Semi-supervised learning can involve providing all or a portion of training data that is partially labeled to a machine learning module. During semi-supervised learning, supervised learning is used for a portion of labeled training data, and unsupervised learning is used for a portion of unlabeled training data. In an embodiment, reinforcement learning is implemented. Reinforcement learning can involve first providing all or a portion of the training data to a machine learning module and as the machine learning module produces an output, the machine learning module receives a “reward” signal in response to a correct output. Typically, the reward signal is a numerical value and the machine learning module is developed to maximize the numerical value of the reward signal. In addition, reinforcement learning can adopt a value function that provides a numerical value representing an expected total of the numerical values provided by the reward signal over time.

### Transfer Learning

In an embodiment, transfer learning is implemented. Transfer learning techniques can involve providing all or a portion of a first training data to a machine learning module, then, after training on the first training data, providing all or a portion of a second training data. In an example embodiment, a first machine learning module can be pre-trained on data from one or more computing devices. The first trained machine learning module is then provided to a computing device, where the computing device is intended to execute the first trained machine learning model to produce an output. Then, during the second training phase, the first trained machine learning model can be additionally trained using additional training data, where the training data can be derived from kernel and non-kernel data of one or more computing devices. This second training of the machine learning module and/or the first trained machine learning model using the training data can be performed using either supervised, unsupervised, or semi-supervised learning. In addition, it is understood transfer learning techniques can involve one, two, three, or more training attempts. Once the machine learning module has been trained on at least the training data, the training phase can be completed. The resulting trained machine learning model can be utilized as at least one of trained machine learning module.

### Incremental and Curriculum Learning

In an embodiment, incremental learning is implemented. Incremental learning techniques can involve providing a trained machine learning module with input data that is used to continuously extend the knowledge of the trained machine learning module. Another machine learning training technique is curriculum learning, which can involve training the machine learning module with training data arranged in a particular order, such as providing relatively easy training examples first, then proceeding with progressively more difficult training examples. As the name suggests, difficulty of training data is analogous to a curriculum or course of study at a school.

### Learning to Learn

In an embodiment, learning to learn is implemented. Learning to learn, or meta-learning, comprises, in general, two levels of learning: quick learning of a single task and slower learning across many tasks. For example, a machine learning module is first trained and comprises a first set of parameters or weights. During or after operation of the first trained machine learning module, the parameters or weights are adjusted by the machine learning module. This process occurs iteratively on the success of the machine learning module. In another example, an optimizer, or another machine learning module, is used wherein the output of a first trained machine learning module is fed to an optimizer that constantly learns and returns the final results. Other techniques for training the machine learning module and/or trained machine learning module are possible as well.

### Contrastive Learning

In example embodiment, contrastive learning is implemented. Contrastive learning is a self-supervised model of learning in which training data is unlabeled is considered as a form of learning in-between supervised and unsupervised learning. This method learns by contrastive loss, which separates unrelated (i.e., negative) data pairs and connects related (i.e., positive) data pairs. For example, to create positive and negative data pairs, more than one view of a datapoint, such as rotating an image or using a different time-point of a video, is used as input. Positive and negative pairs are learned by solving dictionary look-up problem. The two views are separated into query and key of a dictionary. A query has a positive match to a key and negative match to all other keys. The machine learning module then learns by connecting queries to their keys and separating queries from their non-keys. A loss function, such as those described herein, is used to minimize the distance between positive data pairs (e.g., a query to its key) while maximizing the distance between negative data points. See e.g., Tian, Yonglong, et al. “What makes for good views for contrastive learning?.” Advances in Neural Information Processing Systems 33 (2020): 6827-6839.

### Pre-Trained Learning

In an example embodiment, the machine learning module is pre-trained. A pre-trained machine learning model is a model that has been previously trained to solve a similar problem. The pre-trained machine learning model is generally pre-trained with similar input data to that of the new problem. A pre-trained machine learning model further trained to solve a new problem is generally referred to as transfer learning, which is described herein. In an embodiment, a pre-trained machine learning model is trained on a large dataset of related information. The pre-trained model is then further trained and tuned for the new problem. Using a pre-trained machine learning module provides the advantage of building a new machine learning module with input neurons/nodes that are already familiar with the input data and are more readily refined to a particular problem. For example, a machine learning module previously trained using one or more target binding oligonucleotides and the one or more target nucleic acid sequences may be further trained to estimate one or more target binding oligonucleotides. See e.g., Diamant N, et al. Patient contrastive learning: A performant, expressive, and practical approach to electrocardiogram modeling. PLoS Comput Biol. 2022 Feb. 14; 18(2):e1009862.

In an embodiment, after the training phase has been completed but before producing predictions expressed as outputs, a trained machine learning module can be provided to a computing device where a trained machine learning module is not already resident, in other words, after training phase has been completed, the trained machine learning module can be downloaded to a computing device. For example, a first computing device storing a trained machine learning module can provide the trained machine learning module to a second computing device. Providing a trained machine learning module to the second computing device may comprise one or more of communicating a copy of trained machine learning module to the second computing device, making a copy of trained machine learning module for the second computing device, providing access to trained machine learning module to the second computing device, and/or otherwise providing the trained machine learning system to the second computing device. In an example embodiment, a trained machine learning module can be used by the second computing device immediately after being provided by the first computing device. In an embodiment, after a trained machine learning module is provided to the second computing device, the trained machine learning module can be installed and/or otherwise prepared for use before the trained machine learning module can be used by the second computing device.

After a machine learning model has been trained it can be used to output, estimate, infer, predict, generate, produce, or determine, for simplicity these terms will collectively be referred to as results. A trained machine learning module can receive input data and operably generate results. As such, the input data can be used as an input to the trained machine learning module for providing corresponding results to kernel components and non-kernel components. For example, a trained machine learning module can generate results in response to requests. In an example embodiment, a trained machine learning module can be executed by a portion of other software. For example, a trained machine learning module can be executed by a result daemon to be readily available to provide results upon request.

In an embodiment, a machine learning module and/or trained machine learning module can be executed and/or accelerated using one or more computer processors and/or on-device co-processors. Such on-device co-processors can speed up training of a machine learning module and/or generation of results. In an embodiment, trained machine learning module can be trained, reside, and execute to provide results on a particular computing device, and/or otherwise can make results for the particular computing device.

Input data can include data from a computing device executing a trained machine learning module and/or input data from one or more computing devices. In an example embodiment, a trained machine learning module can use results as input feedback. A trained machine learning module can also rely on past results as inputs for generating new results. In an example embodiment, input data can comprise one or more target nucleic acid sequences and, when provided to a trained machine learning module, results in output data such as one or more target binding oligonucleotides.

### Algorithms

Different machine-learning algorithms have been contemplated to carry out the embodiments discussed herein. For example, linear regression (LiR), logistic regression (LoR), Bayesian networks (for example, naive-bayes), random forest (RF) (including decision trees), neural networks (NN) (also known as artificial neural networks), matrix factorization, a diffusion model, a autoregression model, a hidden Markov model (HMM), support vector machines (SVM), K-means clustering (KMC), K-nearest neighbor (KNN), a suitable statistical machine learning algorithm, and/or a heuristic machine learning system for classifying or evaluating one or more target binding oligonucleotides and/or the one or more target nucleic acid sequences.

### Linear Regression (LiR)

In an embodiment, linear regression machine learning is implemented. LiR is typically used in machine learning to predict a result through the mathematical relationship between an independent and dependent variable, such as one or more target nucleic acid sequences and one or more target binding oligonucleotides, respectively or one or more target binding oligonucleotides, the one or more target nucleic acid sequences and an activity score, respectively. A simple linear regression model would have one independent variable (x) and one dependent variable (y). A representation of an example mathematical relationship of a simple linear regression model would be y=mx+b. In this example, the machine learning algorithm tries variations of the tuning variables m and b to optimize a line that includes all the given training data.

The tuning variables can be optimized, for example, with a cost function. A cost function takes advantage of the minimization problem to identify the optimal tuning variables. The minimization problem preposes the optimal tuning variable will minimize the error between the predicted outcome and the actual outcome. An example cost function may comprise summing all the square differences between the predicted and actual output values and dividing them by the total number of input values and results in the average square error.

To select new tuning variables to reduce the cost function, the machine learning module may use, for example, gradient descent methods. An example gradient descent method comprises evaluating the partial derivative of the cost function with respect to the tuning variables. The sign and magnitude of the partial derivatives indicate whether the choice of a new tuning variable value will reduce the cost function, thereby optimizing the linear regression algorithm. A new tuning variable value is selected depending on a set threshold. Depending on the machine learning module, a steep or gradual negative slope is selected. Both the cost function and gradient descent can be used with other algorithms and modules mentioned throughout. For the sake of brevity, both the cost function and gradient descent are well known in the art and are applicable to other machine learning algorithms and may not be mentioned with the same detail.

LiR models may have many levels of complexity comprising one or more independent variables. Furthermore, in an LiR function with more than one independent variable, each independent variable may have the same one or more tuning variables or each, separately, may have their own one or more tuning variables. The number of independent variables and tuning variables will be understood to one skilled in the art for the problem being solved. In an example embodiment, one or more target nucleic acid sequences are used as the independent variables to train a LiR machine learning module, which, after training, is used to estimate, for example, one or more target binding oligonucleotides. In an example embodiment, one or more target binding oligonucleotides and the one or more target nucleic acid sequences are used as the independent variables to train a LiR machine learning module, which, after training, is used to estimate, for example, an activity score.

### Logistic Regression (LoR)

In an embodiment, logistic regression machine learning is implemented. Logistic Regression, often considered a LiR type model, is typically used in machine learning to classify information, such as one or more target nucleic acid sequences into categories such as one or more target binding oligonucleotides or one or more target binding oligonucleotides and the one or more target nucleic acid sequences into categories such as an activity score. LoR takes advantage of probability to predict an outcome from input data. However, what makes LoR different from a LiR is that LoR uses a more complex logistic function, for example a sigmoid function. In addition, the cost function can be a sigmoid function limited to a result between 0 and 1. For example, the sigmoid function can be of the form ƒ(x)=1/(1+e^{-x}), where x represents some linear representation of input features and tuning variables. Similar to LiR, the tuning variable(s) of the cost function are optimized (typically by taking the log of some variation of the cost function) such that the result of the cost function, given variable representations of the input features, is a number between 0 and 1, preferably falling on either side of 0.5. As described in LiR, gradient descent may also be used in LoR cost function optimization and is an example of the process. In an example embodiment, one or more target nucleic acid sequences are used as the independent variables to train a LoR machine learning module, which, after training, is used to estimate, for example, one or more target binding oligonucleotides. In an example embodiment, one or more target binding oligonucleotides and the one or more target nucleic acid sequences are used as the independent variables to train a LoR machine learning module, which, after training, is used to estimate, for example, an activity score.

### Bayesian Network

In an embodiment, a Bayesian Network is implemented. BNs are used in machine learning to make predictions through Bayesian inference from probabilistic graphical models. In BNs, input features are mapped onto a directed acyclic graph forming the nodes of the graph. The edges connecting the nodes contain the conditional dependencies between nodes to form a predicative model. For each connected node the probability of the input features resulting in the connected node is learned and forms the predictive mechanism. The nodes may comprise the same, similar or different probability functions to determine movement from one node to another. The nodes of a Bayesian network are conditionally independent of its non-descendants given its parents thus satisfying a local Markov property. This property affords reduced computations in larger networks by simplifying the joint distribution.

There are multiple methods to evaluate the inference, or predictability, in a BN but only two are mentioned for demonstrative purposes. The first method involves computing the joint probability of a particular assignment of values for each variable. The joint probability can be considered the product of each conditional probability and, in some instances, comprises the logarithm of that product. The second method is Markov chain Monte Carlo (MCMC), which can be implemented when the sample size is large. MCMC is a well-known class of sample distribution algorithms and will not be discussed in detail herein.

The assumption of conditional independence of variables forms the basis for Naïve Bayes classifiers. This assumption implies there is no correlation between different input features. As a result, the number of computed probabilities is significantly reduced as well as the computation of the probability normalization. While independence between features is rarely true, this assumption exchanges reduced computations for less accurate predictions, however the predictions are reasonably accurate. In an example embodiment, one or more target nucleic acid sequences are mapped to the BN graph to train the BN machine learning module, which, after training, is used to estimate one or more target binding oligonucleotides. In an example embodiment, one or more target binding oligonucleotides and the one or more target nucleic acid sequences are mapped to the BN graph to train the BN machine learning module, which, after training, is used to estimate activity score.

### Random Forest

In an embodiment, random forest (RF) is implemented. RF consists of an ensemble of decision trees producing individual class predictions. The prevailing prediction from the ensemble of decision trees becomes the RF prediction. Decision trees are branching flowchart-like graphs comprising of the root, nodes, edges/branches, and leaves. The root is the first decision node from which feature information is assessed and from it extends the first set of edges/branches. The edges/branches contain the information of the outcome of a node and pass the information to the next node. The leaf nodes are the terminal nodes that output the prediction. Decision trees can be used for both classification as well as regression and is typically trained using supervised learning methods. Training of a decision tree is sensitive to the training data set. An individual decision tree may become over or under-fit to the training data and result in a poor predictive model. Random forest compensates by using multiple decision trees trained on different data sets. In an example embodiment, one or more target nucleic acid sequences are used to train the nodes of the decision trees of a RF machine learning module, which, after training, is used to estimate one or more target binding oligonucleotides. In an example embodiment, one or more target binding oligonucleotides and the one or more target nucleic acid sequences are used to train the nodes of the decision trees of a RF machine learning module, which, after training, is used to estimate an activity score.

### Gradient Boosting

In an example embodiment, gradient boosting is implemented. Gradient boosting is a method of strengthening the evaluation capability of a decision tree node. In general, a tree is fit on a modified version of an original data set. For example, a decision tree is first trained with equal weights across its nodes. The decision tree is allowed to evaluate data to identify nodes that are less accurate. Another tree is added to the model and the weights of the corresponding underperforming nodes are then modified in the new tree to improve their accuracy. This process is performed iteratively until the accuracy of the model has reached a defined threshold or a defined limit of trees has been reached. Less accurate nodes are identified by the gradient of a loss function. Loss functions must be differentiable such as a linear or logarithmic functions. The modified node weights in the new tree are selected to minimize the gradient of the loss function. In an example embodiment, a decision tree is implemented to determine one or more target binding oligonucleotides and gradient boosting is applied to the tree to improve its ability to accurately determine the one or more target binding oligonucleotides. In an example embodiment, a decision tree is implemented to determine an activity score and gradient boosting is applied to the tree to improve its ability to accurately determine the activity score.

### Neural Networks

In an embodiment, Neural Networks are implemented. NNs are a family of statistical learning models influenced by biological neural networks of the brain. NNs can be trained on a relatively-large dataset (e.g., 50,000 or more) and used to estimate, approximate, or predict an output that depends on a large number of inputs/features. NNs can be envisioned as so-called “neuromorphic” systems of interconnected processor elements, or “neurons”, and exchange electronic signals, or “messages”. Similar to the so-called “plasticity” of synaptic neurotransmitter connections that carry messages between biological neurons, the connections in NNs that carry electronic “messages” between “neurons” are provided with numeric weights that correspond to the strength or weakness of a given connection. The weights can be tuned based on experience, making NNs adaptive to inputs and capable of learning. For example, an NN for generating one or more non-naturally occurring, engineered target binding oligonucleotides is defined by a set of input neurons that can be given input data such as one or more target nucleic acid sequences. The input neuron weighs and transforms the input data and passes the result to other neurons, often referred to as “hidden” neurons. This is repeated until an output neuron is activated. The activated output neuron produces a result. In an example embodiment, one or more target nucleic acid sequences are used to train the neurons in a NN machine learning module, which, after training, is used to estimate one or more target binding oligonucleotides. In an example embodiment, one or more target binding oligonucleotides and the one or more target nucleic acid sequences are used to train the neurons in a NN machine learning module, which, after training, is used to estimate an activity score.

### Deep Learning

In an embodiment, deep learning is implemented. Deep learning expands the neural network by including more layers of neurons. A deep learning module is characterized as having three “macro” layers: (1) an input layer which takes in the input features, and fetches embeddings for the input, (2) one or more intermediate (or hidden) layers which introduces nonlinear neural net transformations to the inputs, and (3) a response layer which transforms the final results of the intermediate layers to the prediction. In an example embodiment, one or more target nucleic acid sequences are used to train the neurons of a deep learning module, which, after training, is used to estimate one or more target binding oligonucleotides. In an example embodiment, one or more target binding oligonucleotides and the one or more target nucleic acid sequences are used to train the neurons of a deep learning module, which, after training, is used to estimate an activity score.

### Generative Adversarial Network

In an embodiment, the machine learning network comprises a generative adversarial network (GAN). A generative model, in general, can summarize the distribution of input variables and generates new input variables in the input distribution. For example, a (GAN) may have learned a distribution, such as a Gaussian distribution, for a variable. The GAN can then summarize the data distribution, and then generate new input variables that fit into the distribution. GANs may include two components: a generator and a discriminator. A generator creates new input variables from the learned space. In particular, the generator takes a, for example, fixed-length random vector, which is drawn from a Gaussian distribution, as input and generates a sample in the domain. The multidimensional vector space forms a compressed representation of the data distribution. A discriminator determines whether an input variable is real (i.e., a user input) or fake (i.e., generated) using a binary class label. In some instance, a discriminator can be used in transfer learning. The discriminator “trains” the generator to produce input variables that are indiscernible from the user input data. See e.g., Goodfellow, I. J.; Pouget-Abadie, J.; Mirza, M.; Xu, B.; Warde-Farley, D.; Ozair, S.; Courville, A.; Bengio, Y. Generative Adversarial Networks. arXiv 2014. In an example embodiment, the input data comprises one or more target nucleic acid sequences and the GAN generates one or more target binding oligonucleotides.

In an embodiment, the generative adversarial network comprises a Wasserstein Generative Adversarial Network (WGAN). A WGAN modifies a GAN by using a critic that scores the realness or fakeness of an input instead of a discriminator that may classify the realness or fakeness of an input. The advantage to a critic over a discriminator may be, for example, minimizing the distance between the distribution of the data observed in the user input dataset and the distribution observed in generated input. In an embodiment, a continuous distribution is desired when a finer sensitivity to the input is needed. In some instance, the critic model comprises a linear activation function. In an example embodiment, the input data comprises one or more target nucleic acid sequences and the WGAN generates one or more target binding oligonucleotides.

### Convolutional Autoencoder

In an embodiment, a convolutional autoencoder (CAE) is implemented. A CAE is a type of neural network and comprises, in general, two main components. First, the convolutional operator that filters an input signal to extract features of the signal. Second, an autoencoder that learns a set of signals from an input and reconstructs the signal into an output. By combining these two components, the CAE learns the optimal filters that minimize reconstruction error resulting an improved output. CAEs are trained to only learn filters capable of feature extraction that can be used to reconstruct the input. Generally, convolutional autoencoders implement unsupervised learning. In an example embodiment, the convolutional autoencoder is a variational convolutional autoencoder. In an example embodiment, features from an one or more target nucleic acid sequences are used as an input signal into a CAE which reconstructs that signal into an output such as a one or more non-naturally occurring, engineered target binding oligonucleotides. In an example embodiment, features from an one or more target binding oligonucleotides and the one or more target nucleic acid sequences are used as an input signal into a CAE which reconstructs that signal into an output such as an activity score.

### Convolutional Neural Network (CNN)

In an embodiment, a convolutional neural network is implemented. CNNs is a class of NNs further attempting to replicate the biological neural networks, but of the animal visual cortex. CNNs process data with a grid pattern to learn spatial hierarchies of features. Wherein NNs are highly connected, sometimes fully connected, CNNs are connected such that neurons corresponding to neighboring data (e.g., pixels) are connected. This significantly reduces the number of weights and calculations each neuron must perform.

In an embodiment, input data, such one or more target nucleic acid sequences, comprises a multidimensional vector. A CNN, typically, comprises three layers: convolution, pooling, and fully connected. The convolution and pooling layers extract features and the fully connected layer combines the extracted features into an output, such as one or more non-naturally occurring, engineered target binding oligonucleotides.

In an embodiment, input data, such as one or more target binding oligonucleotides and the one or more target nucleic acid sequences, comprises a multidimensional vector. A CNN, typically, comprises three layers: convolution, pooling, and fully connected. The convolution and pooling layers extract features and the fully connected layer combines the extracted features into an output, such as an activity score.

In particular, the convolutional layer comprises multiple mathematical operations such as of linear operations, a specialized type being a convolution. The convolutional layer calculates the scalar product between the weights and the region connected to the input volume of the neurons. These computations are performed on kernels, which are reduced dimensions of the input vector. The kernels span the entirety of the input. The rectified linear unit (i.e., ReLu) applies an elementwise activation function (e.g., sigmoid function) on the kernels.

CNNs can optimized with hyperparameters. In general, there three hyperparameters are used: depth, stride, and zero-padding. Depth controls the number of neurons within a layer. Reducing the depth may increase the speed of the CNN but may also reduce the accuracy of the CNN. Stride determines the overlap of the neurons. Zero-padding controls the border padding in the input.

The pooling layer down-samples along the spatial dimensionality of the given input (i.e., convolutional layer output), reducing the number of parameters within that activation. As an example, kernels are reduced to dimensionalities of 2×2 with a stride of 2, which scales the activation map down to 25%. The fully connected layer uses inter-layer-connected neurons (i.e., neurons are only connected to neurons in other layers) to score the activations for classification and/or regression. Extracted features may become hierarchically more complex as one layer feeds its output into the next layer. See O'Shea, K.; Nash, R. An Introduction to Convolutional Neural Networks. arXiv 2015 and Yamashita, R., et al Convolutional neural networks: an overview and application in radiology. Insights Imaging 9, 611-629 (2018).

### Recurrent Neural Network (RNN)

In an embodiment, a recurrent neural network is implemented. RNNs are class of NNs further attempting to replicate the biological neural networks of the brain. RNNs comprise of delay differential equations on sequential data or time series data to replicate the processes and interactions of the human brain. RNNs have “memory” wherein the RNN can take information from prior inputs to influence the current output. RNNs can process variable length sequences of inputs by using their “memory” or internal state information. Where NNs may assume inputs are independent from the outputs, the outputs of RNNs may be dependent on prior elements with the input sequence. For example, input such as one or more target nucleic acid sequences is received by a RNN, which determines one or more target binding oligonucleotides. For example, input such as one or more target binding oligonucleotides and the one or more target nucleic acid sequences is received by a RNN, which determines an activity score. See Sherstinsky, Alex. “Fundamentals of recurrent neural network (RNN) and long short-term memory (LSTM) network.” Physica D: Nonlinear Phenomena 404 (2020): 132306.

### Long Short-Term Memory (LSTM)

In an embodiment, a Long Short-term Memory is implemented. LSTM are a class of RNNs designed to overcome vanishing and exploding gradients. In RNNs, long term dependencies become more difficult to capture because the parameters or weights either do not change with training or fluctuate rapidly. This occurs when the RNN gradient exponentially decreases to zero, resulting in no change to the weights or parameters, or exponentially increases to infinity, resulting in large changes in the weights or parameters. This exponential effect is dependent on the number of layers and multiplicative gradient. LSTM overcomes the vanishing/exploding gradients by implementing “cells” within the hidden layers of the NN. The “cells” comprise three gates: an input gate, an output gate, and a forget gate. The input gate reduces error by controlling relevant inputs to update the current cell state. The output gate reduces error by controlling relevant memory content in the present hidden state. The forget gate reduces error by controlling whether prior cell states are put in “memory” or forgotten. The gates use activation functions to determine whether the data can pass through the gates. While one skilled in the art would recognize the use of any relevant activation function, example activation functions are sigmoid, tanh, and RELU. See Zhu, Xiaodan, et al. “Long short-term memory over recursive structures.” International Conference on Machine Learning. PMLR, 2015.

### Diffusion Models

In an embodiment, Diffusion Models (DMs) are implemented. A DM, which can also be referred to as a diffusion probabilistic model or score-based generative model, is a type of generative model/latent variable model. Generative models are used to create (i.e., generate) a target variable from an observable variable given a joint probability distribution. A DM includes three (3) main components: the forward module (i.e., diffusion module), the reverse module (i.e., reverse diffusion module), and the sampling module. The forward module takes the input and perturbs (e.g., adds noise to) the data in a stepwise method. Generally, the input data is an unequal distribution data, and the forward module perturbs this distribution until the input data resembles a balanced distribution of data (e.g., Gaussian distribution, can be isotropic or non-isotropic). Data perturbation can include the addition of data, removal of data, shifting/rearrangement of data, or combination thereof. The forward module can either perturb the input data in discrete or continuous steps. In an embodiment, the perturbation to the input data is performed randomly. The forward module can be configured to apply a particular type of perturbation based on the input data and the amount of perturbation per step. It is understood by one of ordinary skill in the art that the configuration is dependent on the input and output. The type of perturbation and the amount of perturbation per step can vary with the application of the described methods and systems. In an embodiment, the configuration is learned (e.g., by a neural network) or empirically designed.

The reverse module then takes the perturbed data as input and trains a denoising or de-perturbation network (e.g., a neural network, an encoder/decoder network,) to learn how to reorganize the balanced distribution of data back into its original form. Data reorganization can include the addition of data, removal of data, shifting/rearrangement of data, or combination thereof. Similar to the forward module, the reverse module reorganizes the distribution of data in stepwise method and the steps can be either discrete or continuous. After training the reverse module can take a perturbed input and generate an output by reorganizing the data according to the learned distribution. Finally, the sampling module uses the denoising/de-perturbation network trained by the reverse module to generate unique outputs from input data.

For example, one or more engineered target binding oligonucleotides is provided as input data to the forward process, which sufficiently perturbs the distribution of sequences of the one or more engineered target binding oligonucleotides. The reverse module then learns how to return the perturbed data back to the original one or more engineered target binding oligonucleotides. The sampling module can then be given one or more target nucleic acid sequences and generate one or more engineered target binding oligonucleotides.

See e.g., Z. Chang, et al., 2023, On the Design Fundamentals of Diffusion Models: A Survey DOI 10.48550/ARXIV.2306.04542.

### Autoregressive Model

In an embodiment, Autoregressive Models (ARs) are implemented. An AR is a type of time series model, which can learn and use relationships between a first set of data (i.e., the input) and a successive set of data (i.e., the output). An AR can include an autocorrelation module (ACP), which describes the successive set of data to the first set of data. The relationship between the first set of data and successive set of data is generally linear, such as the relationship between the one or more target nucleic acid sequences and one or more engineered target binding oligonucleotides, and the AR can be used to model that linear relationship. The AR can be an n-order autoregression models (i.e., wherein n is first-, second-, third-, . . . , n-), such that the output depends on n-related inputs. An AR is generally trained by maximizing the likelihood score between an input and an output using an autoregressive factorization module. Another example of an AR can be described as a Linear Regression Model (LiRM) wherein the dependent variable(s) are the first set of data. The tuning variables are trained to generate the independent variable(s), which is the successive data. The tuning variables determine the strength and direction of the relationship between the first set of data and the successive set of data as well as the reduce background noise/error. ARs described as a LiRM are a unique class of LiRM wherein the dependent variables are constrained to data defined by time series. In an embodiment, the AR is vector autoregression (VAR) model or conditional autoregressive model (CAR) model. In an embodiment, one or more target nucleic acid sequences is processed by an AR to generate one or more engineered target binding oligonucleotides. See e.g., Yang, Z., et al., 2019. Xlnet: Generalized autoregressive pretraining for language understanding. Advances in neural information processing systems, 32.

### Matrix Factorization

In an embodiment, Matrix Factorization is implemented. Matrix factorization machine learning exploits inherent relationships between two entities drawn out when multiplied together. Generally, the input features are mapped to a matrix F which is multiplied with a matrix R containing the relationship between the features and a predicted outcome. The resulting dot product provides the prediction. The matrix R is constructed by assigning random values throughout the matrix. In this example, two training matrices are assembled. The first matrix X contains training input features and the second matrix Z contains the known output of the training input features. First the dot product of R and X are computed and the square mean error, as one example method, of the result is estimated. The values in R are modulated and the process is repeated in a gradient descent style approach until the error is appropriately minimized. The trained matrix R is then used in the machine learning model. In an example embodiment, one or more target nucleic acid sequences are used to train the relationship matrix R in a matrix factorization machine learning module. After training, the relationship matrix R and input matrix F, which comprises vector representations of one or more target nucleic acid sequences, results in the prediction matrix P comprising one or more non-naturally occurring, engineered target binding oligonucleotides. In an example embodiment, one or more target binding oligonucleotides and the one or more target nucleic acid sequences are used to train the relationship matrix R in a matrix factorization machine learning module. After training, the relationship matrix R and input matrix F, which comprises vector representations of one or more target binding oligonucleotides and the one or more target nucleic acid sequences, results in the prediction matrix P comprising an activity score.

### Hidden Markov Model

In an example embodiment, a hidden Markov model is implemented. A HMM takes advantage of the statistical Markov model to predict an outcome. A Markov model assumes a Markov process, wherein the probability of an outcome is solely dependent on the previous event. In the case of HMM, it is assumed an unknown or “hidden” state is dependent on some observable event. A HMM comprises a network of connected nodes. Traversing the network is dependent on three model parameters: start probability; state transition probabilities; and observation probability. The start probability is a variable that governs, from the input node, the most plausible consecutive state. From there each node i has a state transition probability to node j. Typically the state transition probabilities are stored in a matrix Mᵢⱼ wherein the sum of the rows, representing the probability of state i transitioning to state j, equals 1. The observation probability is a variable containing the probability of output o occurring. These too are typically stored in a matrix Nₒⱼ wherein the probability of output o is dependent on state j. To build the model parameters and train the HMM, the state and output probabilities are computed. This can be accomplished with, for example, an inductive algorithm. Next, the state sequences are ranked on probability, which can be accomplished, for example, with the Viterbi algorithm. Finally, the model parameters are modulated to maximize the probability of a certain sequence of observations. This is typically accomplished with an iterative process wherein the neighborhood of states is explored, the probabilities of the state sequences are measured, and model parameters updated to increase the probabilities of the state sequences. In an example embodiment, one or more target nucleic acid sequences are used to train the nodes/states of the HMM machine learning module, which, after training, is used to estimate one or more non-naturally occurring, engineered target binding oligonucleotides. In an example embodiment, one or more target binding oligonucleotides and the one or more target nucleic acid sequences are used to train the nodes/states of the HMM machine learning module, which, after training, is used to estimate an activity score.

### Support Vector Machine

In an example embodiment, support vector machines are implemented. SVMs separate data into classes defined by n-dimensional hyperplanes (n-hyperplane) and are used in both regression and classification problems. Hyperplanes are decision boundaries developed during the training process of a SVM. The dimensionality of a hyperplane depends on the number of input features. For example, a SVM with two input features will have a linear (1-dimensional) hyperplane while a SVM with three input features will have a planer (2-dimensional) hyperplane. A hyperplane is optimized to have the largest margin or spatial distance from the nearest data point for each data type. In the case of simple linear regression and classification a linear equation is used to develop the hyperplane. However, when the features are more complex a kernel is used to describe the hyperplane. A kernel is a function that transforms the input features into higher dimensional space. Kernel functions can be linear, polynomial, a radial distribution function (or gaussian radial distribution function), or sigmoidal. In an example embodiment, one or more target nucleic acid sequences are used to train the linear equation or kernel function of the SVM machine learning module, which, after training, is used to estimate one or more non-naturally occurring, engineered target binding oligonucleotides. In an example embodiment, one or more target binding oligonucleotides and the one or more target nucleic acid sequences are used to train the linear equation or kernel function of the SVM machine learning module, which, after training, is used to estimate an activity score.

### K-Means Clustering

In an embodiment, K-means clustering is implemented. KMC assumes data points have implicit shared characteristics and “clusters” data within a centroid or “mean” of the clustered data points. During training, KMC adds a number of k centroids and optimizes its position around clusters. This process is iterative, where each centroid, initially positioned at random, is re-positioned towards the average point of a cluster. This process concludes when the centroids have reached an optimal position within a cluster. Training of a KMC module is typically unsupervised. In an example embodiment, one or more target nucleic acid sequences are used to train the centroids of a KMC machine learning module, which, after training, is used to estimate one or more non-naturally occurring, engineered target binding oligonucleotides. In an example embodiment, one or more target binding oligonucleotides and the one or more target nucleic acid sequences are used to train the centroids of a KMC machine learning module, which, after training, is used to estimate an activity score.

### K-Nearest Neighbor

In an embodiment, K-nearest neighbor is implemented. On a general level, KNN shares similar characteristics to KMC. For example, KNN assumes data points near each other share similar characteristics and computes the distance between data points to identify those similar characteristics but instead of k centroids, KNN uses k number of neighbors. The k in KNN represents how many neighbors will assign a data point to a class, for classification, or object property value, for regression. Selection of an appropriate number of k is integral to the accuracy of KNN. For example, a large k may reduce random error associated with variance in the data but increase error by ignoring small but significant differences in the data. Therefore, a careful choice of k is selected to balance overfitting and underfitting. Concluding whether some data point belongs to some class or property value k, the distance between neighbors is computed. Common methods to compute this distance are Euclidean, Manhattan or Hamming to name a few. In an embodiment, neighbors are given weights depending on the neighbor distance to scale the similarity between neighbors to reduce the error of edge neighbors of one class “out-voting” near neighbors of another class. In an embodiment, k is 1 and a Markov model approach is utilized. In an example embodiment, one or more target nucleic acid sequences are used to train a KNN machine learning module, which, after training, is used to estimate one or more non-naturally occurring, engineered target binding oligonucleotides. In an example embodiment, one or more target binding oligonucleotides and the one or more target nucleic acid sequences are used to train a KNN machine learning module, which, after training, is used to estimate an activity score.

To perform one or more of its functionalities, the machine learning module may communicate with one or more other systems. For example, an integration system may integrate the machine learning module with one or more email servers, web servers, one or more databases, or other servers, systems, or repositories. In addition, one or more functionalities may require communication between a user and the machine learning module.

Any one or more of the module described herein may be implemented using hardware (e.g., one or more processors of a computer/machine) or a combination of hardware and software. For example, any module described herein may configure a hardware processor (e.g., among one or more hardware processors of a machine) to perform the operations described herein for that module. In some example embodiments, any one or more of the modules described herein may comprise one or more hardware processors and may be configured to perform the operations described herein. In an embodiment, one or more hardware processors are configured to include any one or more of the modules described herein.

Moreover, any two or more of these modules may be combined into a single module, and the functions described herein for a single module may be subdivided among multiple modules. Furthermore, according to various example embodiments, modules described herein as being implemented within a single machine, database, or device may be distributed across multiple machines, databases, or devices. The multiple machines, databases, or devices are communicatively coupled to enable communications between the multiple machines, databases, or devices. The modules themselves are communicatively coupled (e.g., via appropriate interfaces) to each other and to various data sources, to allow information to be passed between the applications so as to allow the applications to share and access common data.

### Multimodal Translation

In an example embodiment, the machine learning module comprises multimodal translation (MT), also known as multimodal machine translation or multimodal neural machine translation. MT comprises a machine learning module capable of receiving multiple (e.g. two or more) modalities. Typically, the multiple modalities comprise of information connected to each other. In an example embodiment, the machine learning module comprising information on one or more target binding oligonucleotides and the one or more target nucleic acid sequences. The machine learning module then determines an activity score corresponding to the one or more target binding oligonucleotides and the one or more target nucleic acid sequences.

In an example embodiment, the MT may comprise of a machine learning method further described herein. In an example embodiment, the MT comprises a neural network, deep neural network, convolutional neural network, convolutional autoencoder, recurrent neural network, or an LSTM. For example, the one or more target binding oligonucleotides and the one or more target nucleic acid sequences is embedded as further described herein. The embedded data is then received by the machine learning module. The machine learning module processes the embedded data (e.g., encoding and decoding) through the multiple layers of architecture then determines the activity score corresponding to the modalities comprising the input. The machine learning methods further described herein may be engineered for MT wherein the inputs described herein comprise of multiple modalities of one or more target binding oligonucleotides and the one or more target nucleic acid sequences. See e.g., Sulubacak, U., Caglayan, O., Grönroos, S A. et al. Multimodal machine translation through visuals and speech. Machine Translation 34, 97-147 (2020) and Huang, Xun, et al. “Multimodal unsupervised image-to-image translation.” Proceedings of the European conference on computer vision (ECCV). 2018.

### Embedding

In an embodiment, the machine learning module may use embedding to provide a lower dimensional representation, such as a vector, of features to organize them based on respective similarities. In some situations, these vectors can become massive. In the case of massive vectors, particular values may become very sparse among a large number of values (e.g., a single instance of a value among 50,000 values). Because such vectors are difficult to work with, reducing the size of the vectors, in some instances, is necessary. A machine learning module can learn the embeddings along with the model parameters. In an example embodiment features such as one or more target binding oligonucleotides and/or one or more target nucleic acid sequences can be mapped to vectors implemented in embedding methods. In an example embodiment, embedded semantic meanings are utilized. Embedded semantic meanings are values of respective similarity. For example, the distance between two vectors, in vector space, may imply two values located elsewhere with the same distance are categorically similar. Embedded semantic meanings can be used with similarity analysis to rapidly return similar values. In an example embodiment, one or more target binding oligonucleotides and/or one or more target nucleic acid sequences is embedded. In an example embodiment, the methods herein are developed to identify meaningful portions of the vector and extract semantic meanings between that space.

### Output of Target Binding Oligonucleotides

In block 230, the oligonucleotide generating system 133 outputs a target binding oligonucleotide or set of target binding oligonucleotides generated in block 220. The output of block 220 can undergo further processing wherein the output data from the machine learning system is processed into user comprehensible information comprising one or more target binding oligonucleotides including one or more mismatches. For example, the output can include a matrix or vector representation, hereafter referred to as matrix representation, of the generated one or more target binding oligonucleotides. The matrix representation can be transformed into a user comprehensible string of the one or more target binding oligonucleotides. The matrix representation can include additional contexts such as increased matrix size, dimensionality, or both. The additional context can be removed after the output is generated. The matrix representation of the one or more target binding oligonucleotides can then be transformed back into a user comprehensible string.

In an embodiment, the one or more target binding oligonucleotides are transmitted back to the user via the network 105. In an example embodiment, the resulting user information is stored on the data storage unit 137. In an example embodiment, the resulting user information is immediately transmitted to the user's device. In an example embodiment, the resulting user information is transmitted across the network 105 to the data acquisition system for subsequent access by the user associated device 110 or oligonucleotide generating network 130.

### Type of Target Binding Oligonucleotides

Target binding oligonucleotides are designed by the methods disclosed herein for binding to one or more target nucleic acid sequences as described above. In an embodiment, the target binding oligonucleotides are non-naturally occurring sequences that differ from the target nucleic acid sequences by one or more mismatches. In an example embodiment, the target binding oligonucleotide is a guide nucleic acid sequence, an RNAi oligonucleotide, a primer nucleic acid sequence, or a probe nucleic acid sequence. The methods disclosed herein may further include a step of synthesizing the target binding oligonucleotides output by methods disclosed herein.

In an embodiment, the target binding oligonucleotide comprises a tag adjacent mismatch. As used herein, a ‘tag adjacent mismatch’ or ‘TAM’ can refer to an introduced mismatch at the terminal position of the protospacer sequence (e.g., position 28 of a 28-nucleotide protospacer, corresponding to position 1 of the spacer sequence) in a guide nucleic acid sequence, wherein said mismatch is positioned immediately adjacent to the direct repeat tag sequence of the guide molecule. The TAM comprises a nucleotide that is non-complementary to the corresponding nucleotide in the target nucleic acid sequence at this position. In an embodiment, the TAM is particularly beneficial when the target nucleic acid sequence contains a guanine (G) nucleotide at the protospacer-flanking site (PFS), as the TAM can disrupt base pairing interactions between the target's anti-tag region and the guide's tag sequence, thereby enhancing guide-target activity.

### Modifications

The target binding oligonucleotides of the invention may comprise modified (i.e., non-naturally occurring) nucleic acids and/or nucleotides and/or nucleotide analogs, and/or chemical modifications. The target binding oligonucleotide can include, for example, mixtures of natural and modified nucleotides. Modified nucleotides and/or nucleotide analogs may be modified at the ribose, phosphate, and/or base moiety. In an embodiment of the invention, a target binding oligonucleotide comprises ribonucleotides and non-ribonucleotides. In an embodiment, a target binding oligonucleotide comprises one or more ribonucleotides and one or more deoxyribonucleotides. In an embodiment of the invention, the target binding oligonucleotide comprises one or more artificial nucleotide or nucleotide analogs such as a nucleotide with phosphorothioate linkage, boranophosphate linkage, a locked nucleic acid (LNA) nucleotide comprising a methylene bridge between the 2′ and 4′ carbons of the ribose ring, or bridged nucleic acids (BNA). Other examples of modified nucleotides include 2′-O-methyl analogs, 2′-deoxy analogs, 2-thiouridine analogs, N6-methyladenosine analogs, or 2-fluoro analogs. Further examples of modified bases include, but are not limited to, 2-aminopurine, 5-bromo-uridine, pseudouridine (Ψ), N¹-methylpseudouridine (me¹Ψ), 5-methoxyuridine (5moU), inosine, 7-methylguanosine. Examples of guide RNA chemical modifications include, without limitation, incorporation of 2′-O-methyl (M), 2′-O-methyl-3′-phosphorothioate (MS), phosphorothioate (PS), S-constrained ethyl (cEt), or 2′-O-methyl-3′-thioPACE (MSP) at one or more terminal nucleotides.

### Guide Nucleic Acid Sequence

In an example embodiment, the target binding oligonucleotide is a guide nucleic acid sequence. The terms guide nucleic acid sequence, guide sequence, and guide molecules are used herein interchangeably. Guides comprise a guide sequence and a scaffold. The scaffold may remain constant for use with a particular Cas protein, while the guide sequence is an engineered sequence designed to change the target nucleic acid sequence recognized by a CRISPR-Cas complex to a target nucleic acid sequence other than a sequence defined by the protospacer of a naturally occurring crRNA. Accordingly, In an embodiment, the target binding oligonucleotide corresponds to the spacer component of the overall guide molecule.

The target binding oligonucleotide spacer component generated by the methods disclosed herein may be used with one or more modifications to the guide molecule. Many modifications to guide molecules are known in the art and are further contemplated within the context of this invention. Various modifications may be used to increase the specificity of binding to the target nucleic acid sequence and/or increase the activity of the Cas protein and/or reduce off-target effects. Example guide molecule modifications are described in International Patent Application No. PCT US2019/045582, specifically paragraphs [0178]-[0333], which is incorporated herein by reference as if expressed in its entirety herein. In an embodiment, the guide molecule is generated to minimize or reduce off-target effects. Guide sequences and strategies to minimize toxicity and off-target effects can be as in WO 2014/093622 (PCT/US2013/074667); or, via mutation as described herein and can be used to train the machine learning models to learn these strategies.

In an embodiment, 5′ and/or 3′ end of a guide molecule is modified by a variety of functional moieties including fluorescent dyes, polyethylene glycol, cholesterol, proteins, or detection tags. (See Kelly et al., 2016, J. Biotech. 233:74-83). Chemical modification in 5′-handle of the stem-loop region of a guide may abolish its function (see Li, et al., Nature Biomedical Engineering, 2017, 1:0066). In an embodiment, three to five nucleotides at 5′ and/or 3′ end of the guide are chemically modified with 2′-O-methyl (M), 2′-O-methyl-3′-phosphorothioate (MS), S-constrained ethyl (cEt), or 2′-O-methyl-3′-thioPACE (MSP). Such modification can enhance genome editing efficiency (see Hendel et al., Nat. Biotechnol. (2015) 33(9): 985-989). In an embodiment, all of the phosphodiester bonds of a guide are substituted with phosphorothioates (PS) for enhancing levels of gene disruption. In an embodiment, more than five nucleotides at the 5′ and/or 3′ end of the guide are chemically modified with 2′-O-Me, 2′-F or S-constrained ethyl (cEt). Such chemically modified guides can mediate enhanced levels of gene disruption (see Rahdar et al., 2015, PNAS, E7110-E7111). In an embodiment, the chemical moiety of the modified guide can be used to attach the guide to another molecule, such as DNA, RNA, protein, or nanoparticles. Such chemically modified guides can be used to identify or enrich cells genetically edited by a CRISPR system (see Lee et al., eLife, 2017, 6:e25312, DOI: 10.7554). In an embodiment, the guide sequence comprises a mixture of RNA and DNA. The partial replacement of RNA nucleotides with DNA nucleotides has been shown to enhance CRISPR-Cas specificity by reducing off-target effects. See Rueda et al., Nat Commun 8, 1610 (2017), DOI: 10.1038/s41467-017-01732-9; Kartje et al., Biochemistry 2018, 57, 21, 3027-3031, DOI: 10.1021/acs.biochem.8b00107; and Yin et al., Nat Chem Biol. 2018 March; 14(3): 311-316, DOI: 10.1038/nchembio.2559. In an embodiment, use is made of a truncated guide (tru-guide), i.e., a guide molecule that comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA.

In an embodiment, one or more portions of a guide molecule can be chemically synthesized. In an embodiment, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317:3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In an embodiment, the invention provides guide molecules that are generated in a manner that allows for the formation of the CRISPR Cas complex and successful binding to the target, while at the same time, not either allowing for or not allowing for successful nuclease activity (i.e., without nuclease activity/without indel activity). Such modified guide molecules are referred to as “dead guides” or “dead guide molecules”. These dead guide molecules can be thought of as catalytically inactive or conformationally inactive with regard to nuclease activity.

### SiRNA

In an embodiment, the target binding oligonucleotides are an siRNA. Small interfering RNA (siRNA) generally comprises a single stranded RNA complementary to a target mRNA. Naturally occurring siRNA is a component of the RNA interference (RNAi) pathway and interferes with the expression of an encoded gene. siRNA binds to the target mRNA and induces mRNA cleavage thereby silencing the gene. Generally, siRNA is approximately 20-24 base pairs long, but may comprise any length required, and may further comprise phosphorylated 5′ ends and hydroxylated 3′ ends with two overhanging nucleotides. See e.g., Lam, J. K. W.; Chow, M. Y. T.; Zhang, Y.; Leung, S. W. S. siRNA Versus miRNA as Therapeutics for Gene Silencing. Molecular Therapy-Nucleic Acids, 2015, 4, e252.

miRNA

In an embodiment, the target binding oligonucleotides are microRNAs. MicroRNAs (miRNAs) are, typically, small RNAs (˜22 nucleotides in length, but may be any necessary length) that target 3′ UTR, 5′ UTR, coding sequence, and gene promoters of a mRNA. miRNA regulates gene expression by, for example, inhibiting or promoting (e.g., up-regulating) gene expression. In an example embodiment, the target binding oligonucleotide comprises miRNA. In an example embodiment, the miRNA is modified. Modified miRNA may comprise phosphorothioate nucleotides and/or 2′-O-methyl additions. Phosphorothioate nucleotides comprise substituting non-bridging oxygen in a phosphate group for sulfur. In an example embodiment, the target binding oligonucleotide is a microRNA inhibitor (antimiR). AntimiRs are nucleic acid sequences complementary to a target miRNA and suppress their function. See e.g., Rupaimoole, Rajesha, and Frank J. Slack. “MicroRNA therapeutics: towards a new era for the management of cancer and other diseases” Nature reviews Drug discovery 16.3 (2017): 203-222, hereby incorporated by reference.

### Primer Nucleic Acid Sequence

In an embodiment, the target binding oligonucleotide is a primer nucleic acid sequence. Primer nucleic acid sequence, as described herein can include any primer nucleic acid sequence that is designed to bind to one or more target nucleic acid sequences and used for amplification of the target nucleic sequence, for example, for use in PCR and other related amplification techniques.

### Probe Nucleic Acid Sequence.

In an example embodiment, the target binding oligonucleotide is a probe nucleic acid sequence. Probe nucleic acid sequence may be RNA or DNA oligonucleotides or their analogs. Probe nucleic acid sequences bind to nucleotide sequences in targeted nucleic acids (analytes) to form probe-analyte hybrids. A stable hybrid may reveal the presence of a target DNA or RNA fragment complementary to the probe nucleic acid sequence.

### Locked Nucleic Acid (LNA)

In an example embodiment, the target binding oligonucleotide is a locked nucleic acid (LNA). Locked nucleotides are characterized by an internal bond between the O2′ and the C4′ of the furanose ring, linked by a methylene group. The modification introduces a conformational lock in the molecule, which nonetheless still retains the physical properties of the native nucleic acid. Two interesting properties of LNAs are advantageous for this application: the enhanced thermal stability of the LNA monomers and their ability to anneal strongly to the untemplated 3′ extension of the cDNA.

### Peptide Nucleic Acid (PNA)

In an example embodiment, the target binding oligonucleotide is a peptide nucleic acid (LNA). PNAs are DNA analogs with neutral synthetic backbone in place of the negatively charged phosphodiester backbone of DNA. This neutral charge allows high-affinity binding to DNA compared to those attained by DNA/DNA or DNA/RNA hybrids. Second, next-generation PNAs (e.g., γPNA) are preorganized for binding to B-DNA in a sequence-unrestricted manner via Watson-Crick recognition. Third, the synthetic backbone of PNAs makes them resistant to proteases/nucleases. Fourth, a PNA/DNA mismatch is more destabilizing than a DNA/RNA mismatch, which could potentially reduce the off-target effects. Finally, efficient in vivo delivery of PNAs has been demonstrated for several disease systems by many groups.

### RNA Toehold Switches

In an embodiment, the target binding oligonucleotide may be a RNA toehold switch. Toehold switches are riboregulators that can activate gene expression in response to cognate RNAs, with a cis-repressing switch RNA hairpin and a trans-acting trigger RNA. Two hairpin switches can also be designed according to current methods, with optimization of spacing from hairpin-to hairpin, as well as binding sites of triggers. The toehold switches can be unfolded upon binding a trigger RNA and are useful in detecting a variety of targets including viral targets such as Zika and Ebola. Toehold switches are sensitive to sequence variations between it and the trigger RNA, with design requiring sequence properties, structures, and specificities to be considered, and particularly suited for the methods disclosed herein.

### Additional Processes

Referring to FIG. 22, and continuing to refer to FIG. 1 for context, a block flow diagram illustrates method 300, in accordance with certain examples of the technology disclosed herein. Either during or after method 200, method 300 can be implemented to measure the biological activity of the designed target binding oligonucleotides. In an example embodiment, method 300 can be implemented immediately after block 210 or any time after block 220 in method 200.

In an example embodiment, a biological activity network the receives an input of one or more target binding oligonucleotides and the one or more target nucleic acid sequences. The biological activity network, generally, is part of the oligonucleotide generating network 130 and for the purpose of this example will be considered as part of the oligonucleotide generating network 130 but may and can be a separate network with the same or similar structure as the oligonucleotide generating network 130. The oligonucleotide generating network 130 may receive the one or more target binding oligonucleotides and the one or more target nucleic acid sequences from the user computing device 110, the data acquisition system 120, or any other suitable source of target binding oligonucleotides via the network 105 to the oligonucleotide generating network 130, discussed in more detail in other sections herein. The acquisition engine comprises any software or hardware individually or in combination described herein that is capable of communicating with a user device, such as fetching, receiving, or sending information, thereby allowing access to the one or more target binding oligonucleotides, the one or more target nucleic acid sequences, and/or activity score by the oligonucleotide generating network 130 or the data acquisition system 120. The one or more target binding oligonucleotides and the one or more target nucleic acid sequences is transferred over a network via the transfer engine from the user associated device 100 or the data acquisition system 120 to the oligonucleotide generating network 130. The transfer engine comprises any software or hardware individually or in combination described herein that is capable of moving or transferring the one or more target binding oligonucleotides and the one or more target nucleic acid sequences thereby allowing access within the oligonucleotide generating network 130.

In block 310, the oligonucleotide generating system 133 processes the data of the one or more target binding oligonucleotides and the one or more target nucleic acid sequences. In an example embodiment, the one or more target binding oligonucleotides and the one or more target nucleic acid sequences is processed with one or more of the machine learning methods described herein. The one or more target binding oligonucleotides and the one or more target nucleic acid sequences is performed by the machine learning algorithm based on data collected by the data acquisition system 120. Accordingly, human analysis or cataloging is not required. The process is performed automatically by the oligonucleotide generating network 130 without human intervention, as described in the machine learning section below. The amount of data typically collected includes thousands to tens of thousands of data items for one or more target binding oligonucleotides and the one or more target nucleic acid sequences. The total number of users may include all users accessing the system or a portion of users using a particular aspect of the system (e.g., the portion of users using the mobile application as opposed to those using a web-browser portal). Human intervention in the process is not useful or required because the amount of data is too great. A team of humans would not be able to catalog or analyze the data in any useful manner. Moreover, a human cannot access one or more target binding oligonucleotides and the one or more target nucleic acid sequences and from that data predict an activity score in a reasonable time to carry out the method.

In block 320, the oligonucleotide generating system 133 generates an activity score for the one or more target binding oligonucleotides and the one or more target nucleic acid sequences. In an embodiment, the oligonucleotide generating system 133 processes the wherein the output data into user comprehensible information comprising the activity score.

### Activity Score

In an example embodiment, an activity score is generated for the one or more target binding oligonucleotides and the one or more target nucleic acid sequences. An activity score may describe various molecular properties and structure features which determine whether one or more target binding oligonucleotides will bind to one or more target nucleic acid sequences. The biological activity network computes properties such as hydrophobicity, electronic distribution, hydrogen bonding characteristics, molecule size and flexibility and of course presence of various pharmacophoric features from the one or more target binding oligonucleotides and the one or more target nucleic acid sequences, which may influence the binding of the one or more target binding oligonucleotides and the one or more target nucleic acid sequences. The biological activity network learns these properties and features through training and, in general, inherently computes these properties and features from the nucleic acid sequences.

In an example embodiment, the activity score generates an active or inactive score. For example, the activity score is a continuous range of activity from most active to least active (i.e., inaction). An active score or inactive score may be a distribution of values over a continuous range, wherein, for example, the distribution is above or below, respectively, a predetermined threshold determines the activity. In another example, the activity score is a single variable on a quantized scale, wherein the activity (i.e., active or inactive) depends on whether the single variable is above or below a predetermined threshold.

In an example embodiment, the activity score is a level of activity of target binding oligonucleotides. The level of activity may include, for example, the probability of binding of the one or more target binding oligonucleotides and the one or more target nucleic acid sequences. The level of activity may include, for example, the amount (e.g., number, ratio, percentage) of binding interactions between the one or more target binding oligonucleotides and the one or more target nucleic acid sequences. The level of activity may include, for example, the binding strength of the one or more target binding oligonucleotides and the one or more target nucleic acid sequences.

In block 330, the activity score is transmitted back to the user via the network 105. In an example embodiment, the resulting user information (i.e., activity score) is stored on the data storage unit 137. In an example embodiment, the resulting user information is immediately transmitted to the user's device. In an example embodiment, the resulting user information is transmitted across the network 105 to the data acquisition system for subsequent access by the user associated device 110 or oligonucleotide generating network 130.

The ladder diagrams, scenarios, flowcharts and block diagrams in the figures and discussed herein illustrate architecture, functionality, and operation of example embodiments and various aspects of systems, methods, and computer program products of the present invention. Each block in the flowchart or block diagrams can represent the processing of information and/or transmission of information corresponding to circuitry that can be configured to execute the logical functions of the present techniques. Each block in the flowchart or block diagrams can represent a module, segment, or portion of one or more executable instructions for implementing the specified operation or step. In an example embodiment, the functions/acts in a block can occur out of the order shown in the figures and nothing requires that the operations be performed in the order illustrated. For example, two blocks shown in succession can executed concurrently or essentially concurrently. In another example, blocks can be executed in the reverse order. Furthermore, variations, modifications, substitutions, additions, or reduction in blocks and/or functions may be used with any of the ladder diagrams, scenarios, flow charts and block diagrams discussed herein, all of which are explicitly contemplated herein.

The ladder diagrams, scenarios, flow charts and block diagrams may be combined with one another, in part or in whole. Coordination will depend upon the required functionality. Each block of the block diagrams and/or flowchart illustration as well as combinations of blocks in the block diagrams and/or flowchart illustrations can be implemented by special purpose hardware-based systems that perform the aforementioned functions/acts or carry out combinations of special purpose hardware and computer instructions. Moreover, a block may represent one or more information transmissions and may correspond to information transmissions among software and/or hardware modules in the same physical device and/or hardware modules in different physical devices.

The present techniques can be implemented as a system, a method, a computer program product, digital electronic circuitry, and/or in computer hardware, firmware, software, or in combinations of them. The system may comprise distinct software modules embodied on a computer readable storage medium; the modules can include, for example, any or all of the appropriate elements depicted in the block diagrams and/or described herein; by way of example and not limitation, any one, some, or all of the modules/blocks and or sub-modules/sub-blocks described. The method steps can then be carried out using the distinct software modules and/or sub-modules of the system, as described above, executing on one or more hardware processors such as CPU or GPU.

The computer program product can include a program tangibly embodied in an information carrier (e.g., computer readable storage medium or media) having computer readable program instructions thereon for execution by, or to control the operation of, data processing apparatus (e.g., a processor) to carry out aspects of one or more embodiments of the present invention. It will be understood that each block of the flowchart illustrations and/or block diagrams, and combinations of blocks in the flowchart illustrations and/or block diagrams, can be implemented by computer readable program instructions.

The computer readable program instructions can be performed on general purpose computing device, special purpose computing device, or other programmable data processing apparatus to produce a machine, such that the instructions, which execute via, at least partially, by one or more processors that are temporarily configured (e.g., by software) or permanently configured to perform the functions/acts specified in the flowchart and/or block diagram block or blocks. The processors, either: temporarily or permanently; or partially configured, may comprise processor-implemented modules. The present techniques referred to herein may, in an example embodiment, comprise processor-implemented modules. Functions/acts of the processor-implemented modules may be distributed among the one or more processors. Moreover, the functions/acts of the processor-implements modules may be deployed across a number of machines, where the machines may be located in a single geographical location or distributed across a number of geographical locations.

The computer readable program instructions can also be stored in a computer readable storage medium that can direct one or more computer devices, programmable data processing apparatuses, and/or other devices to carry out the function/acts of the processor-implemented modules. The computer readable storage medium containing all or partial processor-implemented modules stored therein, comprises an article of manufacture including instructions which implement aspects, operations, or steps to be performed of the function/act specified in the flowchart and/or block diagram block or blocks.

Computer readable program instructions described herein can be downloaded to a computer readable storage medium within a respective computing/processing devices from a computer readable storage medium. Optionally, the computer readable program instructions can be downloaded to an external computer device or external storage device via a network. A network adapter card or network interface in each computing/processing device can receive computer readable program instructions from the network and forward the computer readable program instructions for permanent or temporary storage in a computer readable storage medium within the respective computing/processing device.

Computer readable program instructions described herein can be assembler instructions, instruction-set-architecture (ISA) instructions, machine instructions, machine dependent instructions, microcode, firmware instructions, state-setting data, or either source code or object code. The computer readable program instructions can be written in any programming language such as compiled or interpreted languages. In addition, the programming language can be object-oriented programming language (e.g., “C++”) or conventional procedural programming languages (e.g. “C”) or any combination thereof may be used to as computer readable program instructions. The computer readable program instructions can be distributed in any form, for example as a stand-alone program, module, subroutine, or other unit suitable for use in a computing environment. The computer readable program instructions can execute entirely on one computer or on multiple computers at one site or across multiple sites connected by a communication network, for example on user's computer, partly on the user's computer, as a stand-alone software package, partly on the user's computer and partly on a remote computer or entirely on a remote computer or server. If the computer readable program instructions are executed entirely remote, then the remote computer can be connected to the user's computer through any type of network or the connection can be made to an external computer. In examples embodiments, electronic circuitry including, but not limited to, programmable logic circuitry, field-programmable gate arrays (FPGA), or programmable logic arrays (PLA) can execute the computer readable program instructions. Electronic circuitry can utilize state information of the computer readable program instructions to personalize the electronic circuitry, to execute functions/acts of one or more embodiments of the present invention.

Example embodiments described herein include logic or a number of components, modules, or mechanisms. Modules may comprise either software modules or hardware-implemented modules. A software module may be code embodied on a non-transitory machine-readable medium or in a transmission signal. A hardware-implemented module is a tangible unit capable of performing certain operations and may be configured or arranged in a certain manner. In an example embodiment, one or more computer systems (e.g., a standalone, client or server computer system) or one or more processors may be configured by software (e.g., an application or application portion) as a hardware-implemented module that operates to perform certain operations as described herein.

In an example embodiment, a hardware-implemented module may be implemented mechanically or electronically. In an example embodiment, hardware-implemented modules may comprise permanently configured dedicated circuitry or logic to execute certain functions/acts such as a special-purpose processor or logic circuitry (e.g., a field programmable gate array (FPGA) or an application-specific integrated circuit (ASIC)). In an example embodiment, hardware-implemented modules may comprise temporary programmable logic or circuitry to perform certain functions/acts. For example, a general-purpose processor or other programmable processor.

The term “hardware-implemented module” encompasses a tangible entity. A tangible entity may be physically constructed, permanently configured, or temporarily or transitorily configured to operate in a certain manner and/or to perform certain functions/acts described herein. Hardware-implemented modules that are temporarily configured need not be configured or instantiated at any one time. For example, if the hardware-implemented modules comprise a general-purpose processor configured using software, then the general-purpose processor may be configured as different hardware-implemented modules at different times.

Hardware-implemented modules can provide, receive, and/or exchange information from/with other hardware-implemented modules. The hardware-implemented modules herein may be communicatively coupled. Multiple hardware-implemented modules operating concurrently, may communicate through signal transmission, for instance appropriate circuits and buses that connect the hardware-implemented modules. Multiple hardware-implemented modules configured or instantiated at different times may communicate through temporarily or permanently archived information, for instance the storage and retrieval of information in memory structures to which the multiple hardware-implemented modules have access. For example, one hardware-implemented module may perform an operation, and store the output of that operation in a memory device to which it is communicatively coupled. Consequently, another hardware-implemented module may, at some time later, access the memory device to retrieve and process the stored information. Hardware-implemented modules may also initiate communications with input or output devices and can operate on information from the input or output devices.

In an example embodiment, the present techniques can be at least partially implemented in a cloud or virtual machine environment.

### ADDITIONAL EXAMPLE EMBODIMENTS

In an embodiment, the oligonucleotide generating network includes one or more machine learning models (e.g., a Diffusion Model, Autoregression Model, Neural Network, etc. . . . ) and takes one or more target nucleic acid sequences as input. The machine learning model (MLM), including an oligonucleotide generating network, can also process a desired target interaction score, non-interaction score, or both as additional input. For example, an MLM including an oligonucleotide generating network is trained on one or more engineered target binding oligonucleotides with a known target interaction score, non-interaction score, or both. After training, a user can use one or more target nucleic acid sequences with a desired target interaction score, non-interaction score, or both as input for the MLM including oligonucleotide generating network to generate one or more engineered target binding oligonucleotides with said target interaction score, non-interaction score, or both. For example, the desired target interaction score, non-interaction score, or both refers to a user-defined value, which will be apparent to the user depending on the application of the methods and systems described herein. By way of an example, for one application, a user may want one or more engineered target-binding oligonucleotides to have one target with high specificity, while for another application, a user may want one or more engineered target-binding oligonucleotides to have more than one target and therefore low or lower specificity.

In an embodiment, the MLM, including an oligonucleotide generating network, after training, can be used to generate one or more engineered target binding oligonucleotides with an optimized target interaction score, an optimized non-interaction score, or both. An optimized target interaction score and an optimized non-interaction score refer to target interaction scores and non-interaction scores with enhanced and/or superior values relative to target interaction scores, a non-interaction scores, or both manually designed by a person. The one or more engineered target binding oligonucleotides with the optimized target interaction score, the optimized non-interaction score, or both can then be used to train an objective network.

In a preferred embodiment, the machine learning program may comprise a Wasserstein generative adversarial network (WGAN). In an example embodiment, the WGAN is conditional on one or more target nucleic acid sequences. In another example embodiment, the oligonucleotide generating system may comprise an evolutionary algorithm. For example, in an embodiment, the evolutionary network comprises a fitness evaluation. In an embodiment, the evolutionary network comprises an objective network. In an embodiment, the fitness evaluation may include a fitness function, fitness landscape, or any combination thereof. In an embodiment, the fitness evaluation is across a multi-dimensional (MD) latent vector. In an embodiment, the (MD) latent vector is 2 or more dimensions, 3 or more dimensions, 4 or more dimensions, 5 or more dimensions, 6 or more dimensions, 7 or more dimensions, 8 or more dimensions, 9 or more dimensions, 10 or more dimensions, 11 or more dimensions, 12 or more dimensions, 13 or more dimensions, 14 or more dimensions, 15 or more dimensions. In an embodiment, the MD latent vector is transformed into a n x m fitness matrix, wherein n is the dimensions of the latent vector and m is any 2 or more dimensions.

In an embodiment, the fitness matrix is processed by a ML model to generate one or more target binding oligonucleotides defined by a maximal fitness. For example, the fitness matrix is passed through three residual modules, with each residual module consisting of two (2) or more layers. Each layer can contain a 1-dimensional or multi-dimensional (e.g., 2, 3, 4, etc.,) convolutional layer with one (1) or more (e.g., 11, 12, 13, 14, 15, 16, 17, etc.,) filters of stride one (1) or more (e.g., 2, 3, 4, 5, 6, 7, 8, 9, 10, etc.,) and width of about 3 or more (e.g., 1, 2, 3, 4, 5, 6, 7, 8, 9, 10, etc.,). In addition, for example, a convolutional layer with about 4 (e.g., 1, 2, 3, 4, 5, 6, 7, 8, 9, 10, etc.,) filters of width 1 (e.g., 1, 2, 3, 4, 5, 6, 7, 8, 9, 10, etc.,) or more and stride 1 or more (e.g., 1, 2, 3, 4, 5, 6, 7, 8, 9, 10, etc.,) can be applied to the output of the residual module to create multi-channel (e.g., 1, 2, 3, 4, 5, 6, 7, 8, 9, 10, etc.,) encoded guide sequence. The matrix can also be cropped to remove the context on each side of the target binding oligonucleotide, and the resulting matrix is passed through a softmax layer so that each element in the matrix represents the probability of having a certain base at that position. The target binding oligonucleotide contains the base at each position with the greatest probability.

In an embodiment, the method further comprises preparing the one or more engineered target binding oligonucleotides.

In an embodiment, the method further comprises transmitting the one or more target binding oligonucleotides and the one or more target nucleic acid sequences to a deployed biological activity network, by one or more computing devices; processing the one or more target binding oligonucleotides and the one or more target nucleic acid sequences with the deployed biological activity network; and generating, by the biological activity network, an activity score for the one or more target binding oligonucleotides and the one or more target nucleic acid sequences, wherein the steps are performed any time after the first step in the method.

In an embodiment, technology includes a method to generate one or more engineered target binding oligonucleotides to operate on user computing devices. The application may be a downloadable application or application programming interface for use on a computing device that generates one or more engineered target binding oligonucleotides. The data may include one or more target nucleic acid sequences data.

In an embodiment, the technology includes applications and systems to generate one or more engineered target binding oligonucleotides. For example, applications may be provided to individual users capable of communicating through wireless means. In an embodiment, technology includes a method of generating an activity score for one or more target binding oligonucleotides and one or more target nucleic acid sequences to operate on user computing devices. The application may be a downloadable application or application programming interface for use on a computing device that generates an activity score for one or more target binding oligonucleotides and one or more target nucleic acid sequences. The data may include one or more target binding oligonucleotides and one or more target nucleic acid sequences data.

In an embodiment, the technology includes applications and systems for generating one or more engineered target-binding oligonucleotides. For example, applications may be provided to individual users capable of communicating through wireless means.

This invention represents an advance in computer engineering that represents a substantial advancement over existing practices of generating one or more engineered target binding oligonucleotides that include one or more mismatch(es). The data acquired to prepare the predictive models are technical data relating to target nucleic acid sequences. Because of the data that is acquired, processed, and categorized, any number of human users would be unable to create the predictive models or perform the operations described herein. The outputs of the machine learning systems are not obtainable by humans or by conventional methods. For example, generating one or more engineered target-binding oligonucleotides that include one or more mismatch(es) can require exploring an enormous high-dimensional space of sequences. Each engineered target-binding oligonucleotide is generated according to the task at hand, such as multi-target detection or variant identification. An engineered target-binding oligonucleotide should be nearby in sequence space to the complements of their targets, but this region can be sufficiently large to motivate the development of algorithms that efficiently search the sequence space. Generating one or more engineered target-binding oligonucleotides comprising one or more mismatches is a non-conventional, technical, real-world output and benefit that is not obtainable with conventional systems. The methods and systems described herein are more consistent, accurate, and efficient than manual/human analysis, which is prone to bias and doesn't scale to the amount of qualitative data that is generated today.

### Example Computing Device

FIG. 3 depicts a block diagram of a computing machine 2000 and a module 2050 in accordance with certain examples. The computing machine 2000 may comprise, but are not limited to, remote devices, work stations, servers, computers, general purpose computers, Internet/web appliances, hand-held devices, wireless devices, portable devices, wearable computers, cellular or mobile phones, personal digital assistants (PDAs), smart phones, smart watches, tablets, ultrabooks, netbooks, laptops, desktops, multi-processor systems, microprocessor-based or programmable consumer electronics, game consoles, set-top boxes, network PCs, mini-computers, and any machine capable of executing the instructions. The module 2050 may comprise one or more hardware or software elements configured to facilitate the computing machine 2000 in performing the various methods and processing functions presented herein. The computing machine 2000 may include various internal or attached components such as a processor 2010, system bus 2020, system memory 2030, storage media 2040, input/output interface 2060, and a network interface 2070 for communicating with a network 2080.

The computing machine 2000 may be implemented as a conventional computer system, an embedded controller, a laptop, a server, a mobile device, a smartphone, a set-top box, a kiosk, a router or other network node, a vehicular information system, one or more processors associated with a television, a customized machine, any other hardware platform, or any combination or multiplicity thereof. The computing machine 2000 may be a distributed system configured to function using multiple computing machines interconnected via a data network or bus system.

The one or more processor 2010 may be configured to execute code or instructions to perform the operations and functionality described herein, manage request flow and address mappings, and to perform calculations and generate commands. Such code or instructions could include, but is not limited to, firmware, resident software, microcode, and the like. The processor 2010 may be configured to monitor and control the operation of the components in the computing machine 2000. The processor 2010 may be a general purpose processor, a processor core, a multiprocessor, a reconfigurable processor, a microcontroller, a digital signal processor (“DSP”), an application specific integrated circuit (“ASIC”), tensor processing units (TPUs), a graphics processing unit (“GPU”), a field programmable gate array (“FPGA”), a programmable logic device (“PLD”), a radio-frequency integrated circuit (RFIC), a controller, a state machine, gated logic, discrete hardware components, any other processing unit, or any combination or multiplicity thereof. In an example embodiment, each processor 2010 can include a reduced instruction set computer (RISC) microprocessor. The processor 2010 may be a single processing unit, multiple processing units, a single processing core, multiple processing cores, special purpose processing cores, co-processors, or any combination thereof. According to certain examples, the processor 2010 along with other components of the computing machine 2000 may be a virtualized computing machine executing within one or more other computing machines. Processors 2010 are coupled to system memory and various other components via a system bus 2020.

The system memory 2030 may include non-volatile memories such as read-only memory (“ROM”), programmable read-only memory (“PROM”), erasable programmable read-only memory (“EPROM”), flash memory, or any other device capable of storing program instructions or data with or without applied power. The system memory 2030 may also include volatile memories such as random-access memory (“RAM”), static random-access memory (“SRAM”), dynamic random-access memory (“DRAM”), and synchronous dynamic random-access memory (“SDRAM”). Other types of RAM also may be used to implement the system memory 2030. The system memory 2030 may be implemented using a single memory module or multiple memory modules. While the system memory 2030 is depicted as being part of the computing machine 2000, one skilled in the art will recognize that the system memory 2030 may be separate from the computing machine 2000 without departing from the scope of the subject technology. It should also be appreciated that the system memory 2030 is coupled to system bus 2020 and can include a basic input/output system (BIOS), which controls certain basic functions of the processor 2010 and/or operate in conjunction with, a non-volatile storage device such as the storage media 2040.

In an example embodiment, the computing device 2000 includes a graphics processing unit (GPU) 2090. Graphics processing unit 2090 is a specialized electronic circuit designed to manipulate and alter memory to accelerate the creation of images in a frame buffer intended for output to a display. In general, a graphics processing unit 2090 is efficient at manipulating computer graphics and image processing and has a highly parallel structure that makes it more effective than general-purpose CPUs for algorithms where processing of large blocks of data is done in parallel.

The storage media 2040 may include a hard disk, a floppy disk, a compact disc read only memory (“CD-ROM”), a digital versatile disc (“DVD”), a Blu-ray disc, a magnetic tape, a flash memory, other non-volatile memory device, a solid state drive (“SSD”), any magnetic storage device, any optical storage device, any electrical storage device, any electromagnetic storage device, any semiconductor storage device, any physical-based storage device, any removable and non-removable media, any other data storage device, or any combination or multiplicity thereof. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any other data storage device, or any combination or multiplicity thereof. The storage media 2040 may store one or more operating systems, application programs and program modules such as module 2050, data, or any other information. The storage media 2040 may be part of, or connected to, the computing machine 2000. The storage media 2040 may also be part of one or more other computing machines that are in communication with the computing machine 2000 such as servers, database servers, cloud storage, network attached storage, and so forth. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

The module 2050 may comprise one or more hardware or software elements, as well as an operating system, configured to facilitate the computing machine 2000 with performing the various methods and processing functions presented herein. The module 2050 may include one or more sequences of instructions stored as software or firmware in association with the system memory 2030, the storage media 2040, or both. The storage media 2040 may therefore represent examples of machine or computer readable media on which instructions or code may be stored for execution by the processor 2010. Machine or computer readable media may generally refer to any medium or media used to provide instructions to the processor 2010. Such machine or computer readable media associated with the module 2050 may comprise a computer software product. Each of the operating system, one or more application programs, other program modules, and program data or some combination thereof, may include an implementation of a networking environment. It should be appreciated that a computer software product comprising the module 2050 may also be associated with one or more processes or methods for delivering the module 2050 to the computing machine 2000 via the network 2080, any signal-bearing medium, or any other communication or delivery technology. The module 2050 may also comprise hardware circuits or information for configuring hardware circuits such as microcode or configuration information for an FPGA or other PLD.

The input/output (“I/O”) interface 2060 may be configured to couple to one or more external devices, to receive data from the one or more external devices, and to send data to the one or more external devices. Such external devices along with the various internal devices may also be known as peripheral devices. The I/O interface 2060 may include both electrical and physical connections for coupling in operation the various peripheral devices to the computing machine 2000 or the processor 2010. The I/O interface 2060 may be configured to communicate data, addresses, and control signals between the peripheral devices, the computing machine 2000, or the processor 2010. The I/O interface 2060 may be configured to implement any standard interface, such as small computer system interface (“SCSI”), serial-attached SCSI (“SAS”), fiber channel, peripheral component interconnect (“PCI”), PCI express (PCIe), serial bus, parallel bus, advanced technology attached (“ATA”), serial ATA (“SATA”), universal serial bus (“USB”), Thunderbolt, FireWire, various video buses, and the like. The I/O interface 2060 may be configured to implement only one interface or bus technology. Alternatively, the I/O interface 2060 may be configured to implement multiple interfaces or bus technologies. The I/O interface 2060 may be configured as part of, all of, or to operate in conjunction with, the system bus 2020. The I/O interface 2060 may include one or more buffers for buffering transmissions between one or more external devices, internal devices, the computing machine 2000, or the processor 2010.

The I/O interface 2060 may couple the computing machine 2000 to various input devices including cursor control devices, touch-screens, scanners, electronic digitizers, sensors, receivers, touchpads, trackballs, cameras, microphones, alphanumeric input devices, any other pointing devices, or any combinations thereof. The I/O interface 2060 may couple the computing machine 2000 to various output devices including video displays (The computing device 2000 may further include a graphics display, for example, a plasma display panel (PDP), a light emitting diode (LED) display, a liquid crystal display (LCD), a projector, a cathode ray tube (CRT), or any other display capable of displaying graphics or video), audio generation device, printers, projectors, tactile feedback devices, automation control, robotic components, actuators, motors, fans, solenoids, valves, pumps, transmitters, signal emitters, lights, and so forth. The I/O interface 2060 may couple the computing device 2000 to various devices capable of input and out, such as a storage unit. The devices can be interconnected to the system bus 2020 via a user interface adapter, which can include, for example, a Super I/O chip integrating multiple device adapters into a single integrated circuit.

The computing machine 2000 may operate in a networked environment using logical connections through the network interface 2070 to one or more other systems or computing machines across the network 2080. The network 2080 may include a local area network (“LAN”), a wide area network (“WAN”), an intranet, an Internet, a mobile telephone network, storage area network (“SAN”), personal area network (“PAN”), a metropolitan area network (“MAN”), a wireless network (“WiFi;”), wireless access networks, a wireless local area network (“WLAN”), a virtual private network (“VPN”), a cellular or other mobile communication network, Bluetooth, near field communication (“NFC”), ultra-wideband, wired networks, telephone networks, optical networks, copper transmission cables, or combinations thereof or any other appropriate architecture or system that facilitates the communication of signals and data. The network 2080 may be packet switched, circuit switched, of any topology, and may use any communication protocol. The network 2080 may comprise routers, firewalls, switches, gateway computers and/or edge servers. Communication links within the network 2080 may involve various digital or analog communication media such as fiber optic cables, free-space optics, waveguides, electrical conductors, wireless links, antennas, radio-frequency communications, and so forth.

Information for facilitating reliable communications can be provided, for example, as packet/message sequencing information, encapsulation headers and/or footers, size/time information, and transmission verification information such as cyclic redundancy check (CRC) and/or parity check values. Communications can be made encoded/encrypted, or otherwise made secure, and/or decrypted/decoded using one or more cryptographic protocols and/or algorithms, such as, but not limited to, Data Encryption Standard (DES), Advanced Encryption Standard (AES), a Rivest-Shamir-Adelman (RSA) algorithm, a Diffie-Hellman algorithm, a secure sockets protocol such as Secure Sockets Layer (SSL) or Transport Layer Security (TLS), and/or Digital Signature Algorithm (DSA). Other cryptographic protocols and/or algorithms can be used as well or in addition to those listed herein to secure and then decrypt/decode communications.

The processor 2010 may be connected to the other elements of the computing machine 2000 or the various peripherals discussed herein through the system bus 2020. The system bus 2020 represents one or more of any of several types of bus structures, including a memory bus or memory controller, a peripheral bus, an accelerated graphics port, and a processor or local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus. It should be appreciated that the system bus 2020 may be within the processor 2010, outside the processor 2010, or both. According to certain examples, any of the processor 2010, the other elements of the computing machine 2000, or the various peripherals discussed herein may be integrated into a single device such as a system on chip (“SOC”), system on package (“SOP”), or ASIC device.

Examples may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing examples in computer programming, and the examples should not be construed as limited to any one set of computer program instructions. Further, a skilled programmer would be able to write such a computer program to implement an example of the disclosed examples based on the appended flow charts and associated description in the application text. Therefore, disclosure of a particular set of program code instructions is not considered necessary for an adequate understanding of how to make and use examples. Further, those ordinarily skilled in the art will appreciate that one or more aspects of examples described herein may be performed by hardware, software, or a combination thereof, as may be embodied in one or more computing systems. Moreover, any reference to an act being performed by a computer should not be construed as being performed by a single computer as more than one computer may perform the act.

The examples described herein can be used with computer hardware and software that perform the methods and processing functions described herein. The systems, methods, and procedures described herein can be embodied in a programmable computer, computer-executable software, or digital circuitry. The software can be stored on computer-readable media. For example, computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (FPGA), etc.

A “server” may comprise a physical data processing system (for example, the computing device 2000 as shown in FIG. 3) running a server program. A physical server may or may not include a display and keyboard. A physical server may be connected, for example by a network, to other computing devices. Servers connected via a network may operate in the capacity of a server machine or a client machine in a server-client network environment, or as a peer machine in a distributed (e.g., peer-to-peer) network environment. The computing device 2000 can include clients' servers. For example, a client and server can be remote from each other and interact through a network. The relationship of client and server arises by virtue of computer programs in communication with each other, running on the respective computers.

Any two or more devices, two or more software/programs, and any two or more portions of a device or software/program, for simplicity referred to as technology, may be described herein as operably linked. Operably linked may be defined as at least one technology can mediate a function exerted upon at least one other technology such that the two or more technologies function normally. In general, operably linked refers to the ability for at least one technology to communicate with at least one other technology.

The example systems, methods, and acts described in the examples and described in the figures presented previously are illustrative, not intended to be exhaustive, and not meant to be limiting. In alternative examples, certain acts can be performed in a different order, in parallel with one another, omitted entirely, and/or combined between different examples, and/or certain additional acts can be performed, without departing from the scope and spirit of various examples. Plural instances may implement components, operations, or structures described as a single instance. Structures and functionality that may appear as separate. In an example embodiment may be implemented as a combined structure or component. Similarly, structures and functionality that may appear as a single component may be implemented as separate components. Accordingly, such alternative examples are included in the scope of the following claims, which are to be accorded the broadest interpretation to encompass such alternate examples. The terminology used herein was chosen to best explain the principles of the embodiments, the practical application or technical improvement over technologies found in the marketplace, or to enable others of ordinary skill in the art to understand the embodiments disclosed herein.

All publications, published patent documents, and patent applications cited herein are hereby incorporated by reference to the same extent as though each individual publication, published patent document, or patent application was specifically and individually indicated as being incorporated by reference.

Further embodiments are illustrated in the following Examples which are given for illustrative purposes only and are not intended to limit the scope of the invention.

### WORKING EXAMPLES

### Example 1—Model-Directed Generation of CRISPR-Cas13a Guide RNAs Designs Artificial Sequences that Improve Nucleic Acid Detection

### Main Text

In this work, Applicants focused on designing guides for use with CRISPR-Cas13a-based detection assays; specifically, Applicants designed artificial 28-nt CRISPR guide RNA spacer sequences. Cas13a-based assays have increasingly received attention because of their minimal equipment requirements and ease of use in low-resource settings^{18, 19}. Applicants previously described ADAPT⁶, which designs diagnostic guides by using a convolutional neural network (CNN) as a discriminative model to select among natural sequences (or consensuses of sequence clusters). Since the approach selects among a predetermined list of sequences, ADAPT cannot design guides that are several mismatches or more from natural sequences; it cannot design guides for variant identification down to single-nucleotide resolution; and it is unable to uncover novel strategies that optimize guide design. By contrast, algorithms that create artificial guides could achieve these goals while enabling superior diagnostic sensitivity and specificity, particularly for challenges in infectious diseases.

Designing artificial guides necessitates exploring a vast high-dimensional space of RNA sequences. Each guide has a fitness according to the task at hand, such as multi-target detection (FIG. 4a) or variant identification (FIG. 4b). Highly fit artificial guides should be nearby in sequence space to the complements of their targets, but this region, with ˜10⁷-10⁹ sequences (Supplementary Note 1), is sufficiently large to motivate the development of algorithms that efficiently search the sequence space.

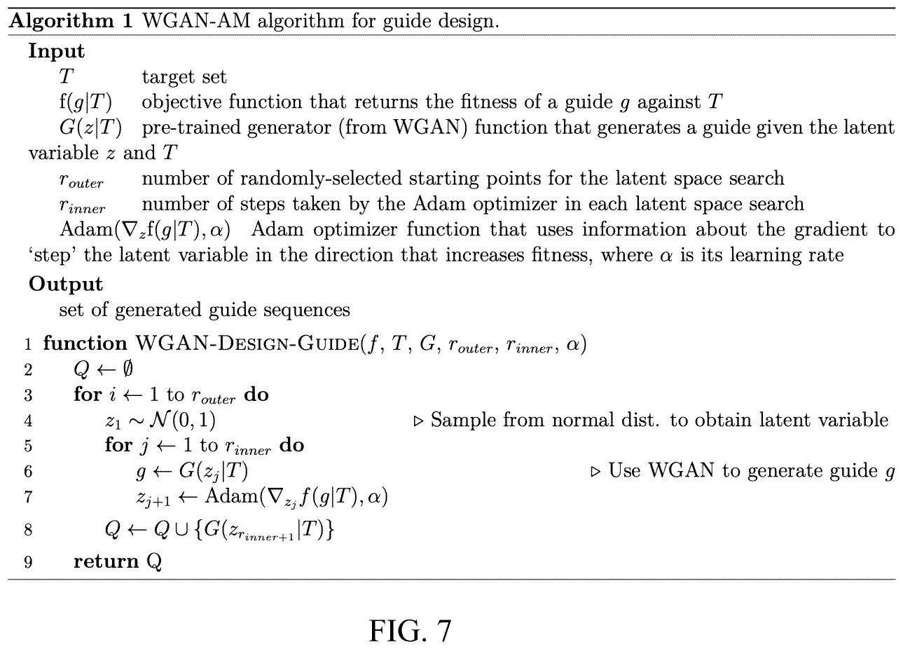

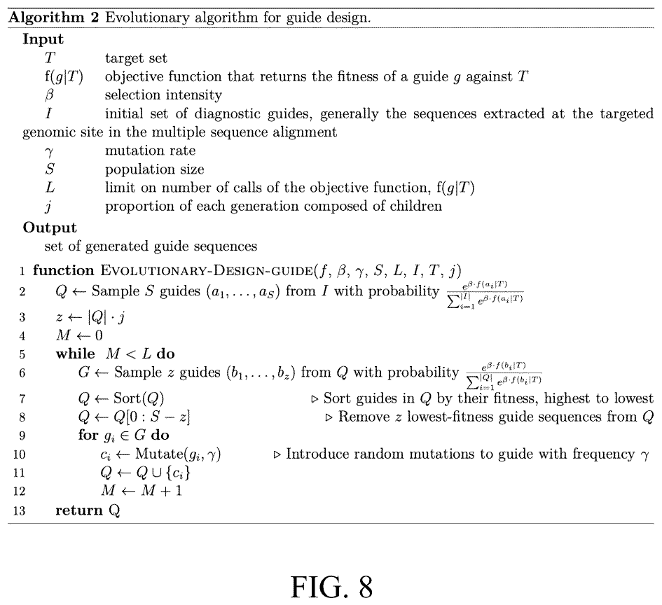

Here, Applicants implemented and evaluated a set of algorithms for designing artificial CRISPR-Cas13a guide RNAs. These algorithms combine a machine-learned model—trained to predict the enzymatic activity that results from a guide-target interaction—with search algorithms to explore a fitness landscape of guides. The predictive model is a CNN that predicts Cas13a's enzymatic activity, a proxy of a given guide's sensitivity for detecting a given target. Applicants implemented and evaluated two design algorithms: (i) a generative model that employs a conditional Wasserstein Generative Adversarial Network and activation maximization to explore a continuous latent space of sequences^{13, 20, 21} (WGAN-AM; FIG. 4c and FIG. 7); (ii) an evolutionary algorithm²²⁻²⁴ that performs iterative rounds of random mutation, fitness evaluation, and selection to explore the fitness landscape in discrete steps (FIG. 4d and FIG. 8). Importantly, the model-directed exploration process—including the predictive model, search algorithms, and fitness functions—is conditioned on user-provided target sequences, making it broadly applicable to any input targets rather than being restricted to specific genomic sites. Applicants built a software package to make these design approaches easily accessible for Cas13 guide RNA design. It is called BADGERS (Building Artificial Diagnostic Guides by Exploring Regions of Sequences) and is available at github.com/broadinstitute/badgers-cas13.

First, Applicants applied the algorithms in BADGERS to design guides, for use with Cas13a-based detection, that are sensitive across a pathogen's genomic variation, i.e., for multi-target detection. The fitness function that Applicants maximized is the average predicted activity across sequenced genomes (FIG. 4a, Methods). Applicants focused on five RNA viruses, chosen for their extensive diversity (enterovirus B, Lassa, dengue) or public health relevance (influenza A, SARS-CoV-2). Applicants employed the design algorithms implemented in BADGERS to design guides and benchmarked them against algorithms representing current, state-of-the-art design approaches (Methods). These baseline methods include a simple, commonly used algorithm that complements the consensus sequence at the target site (“consensus”) and a more advanced algorithm that Applicants term “model-based choice” (MBC), which reflects the method implemented in ADAPT, which Applicants previously developed⁶. MBC employs clustering to compute a candidate set of guides and selects the guide, from this predetermined list, with maximal fitness based on the model's predictions. Although the predictive model of guide activity used by MBC and the algorithms in BADGERS is the same, MBC is conceptually and algorithmically distinct; MBC does not use the predictive model to explore a sequence space, and it cannot generate artificial guides with several mismatches from the targets (Methods).

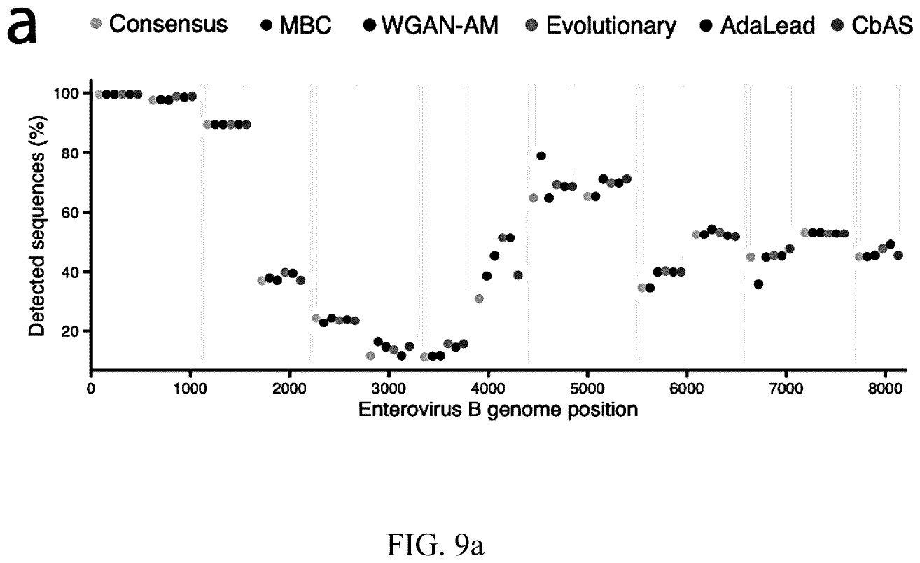

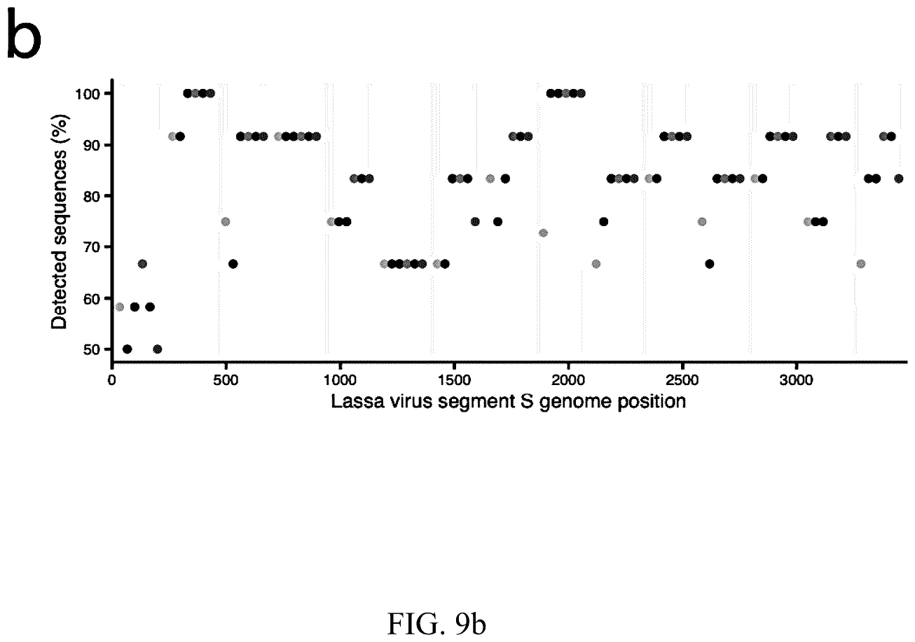

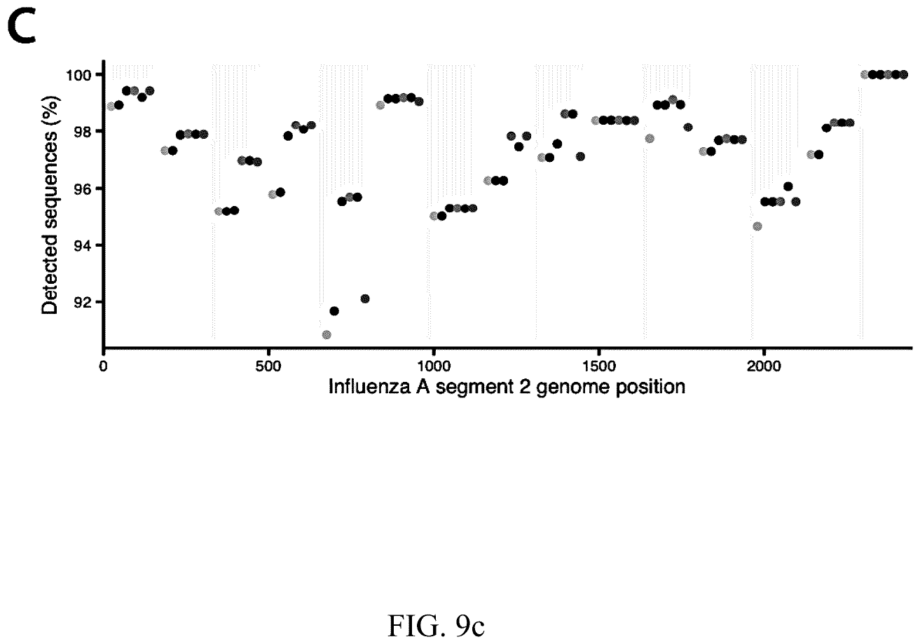

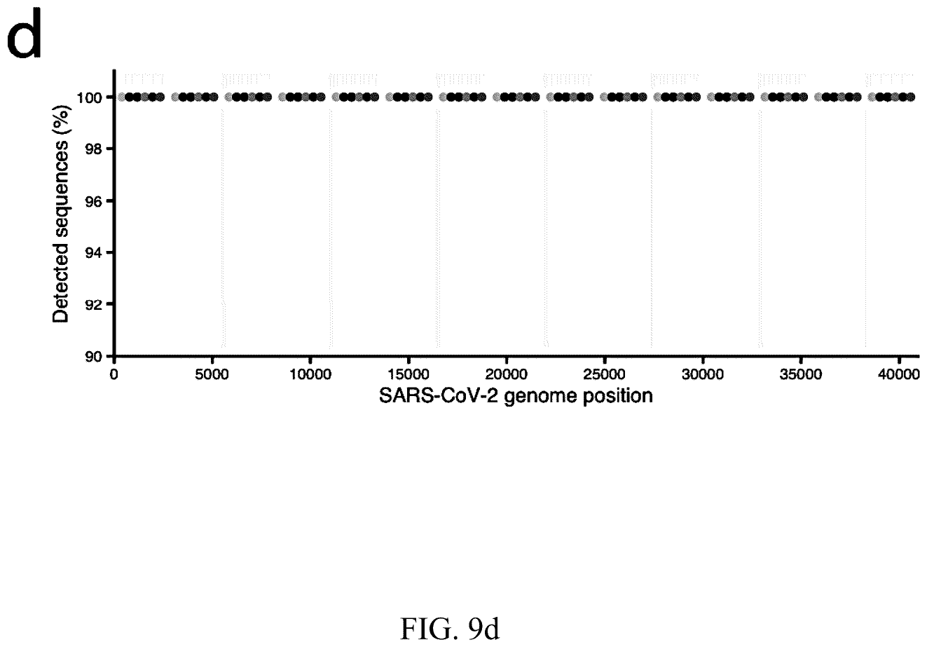

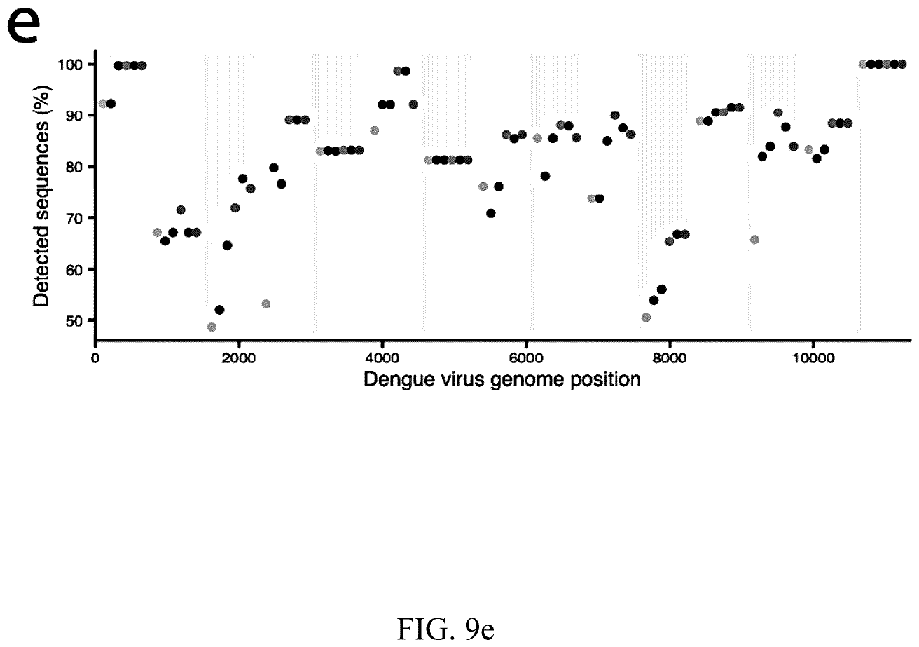

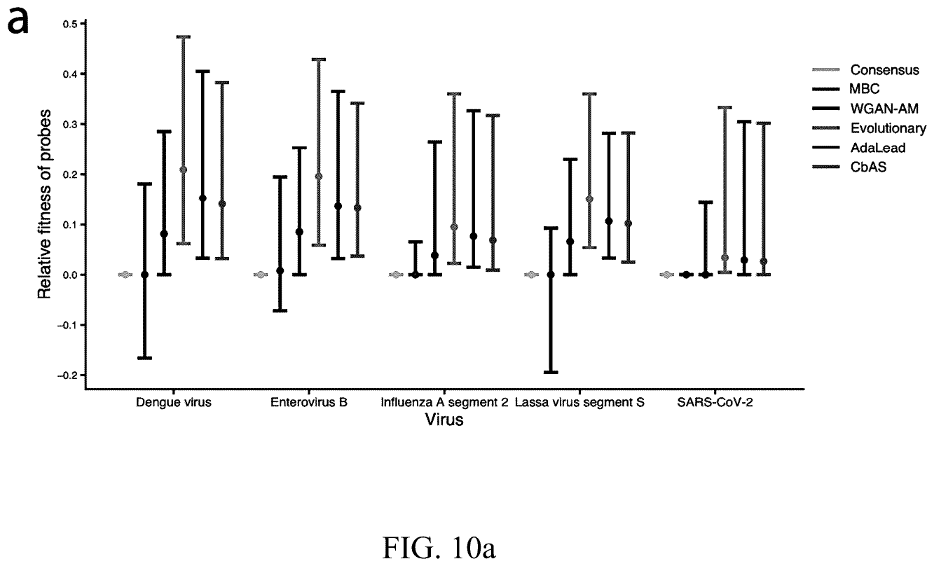

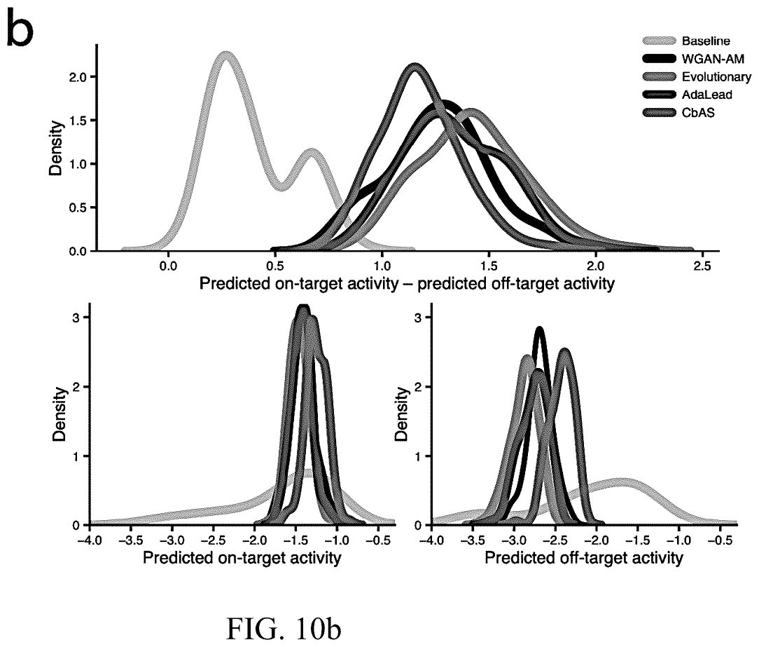

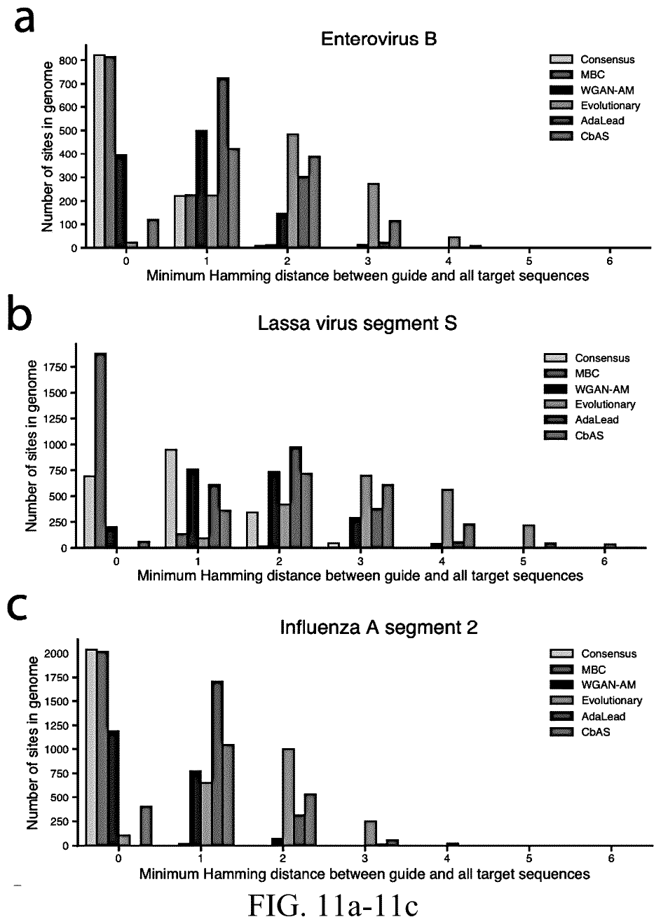

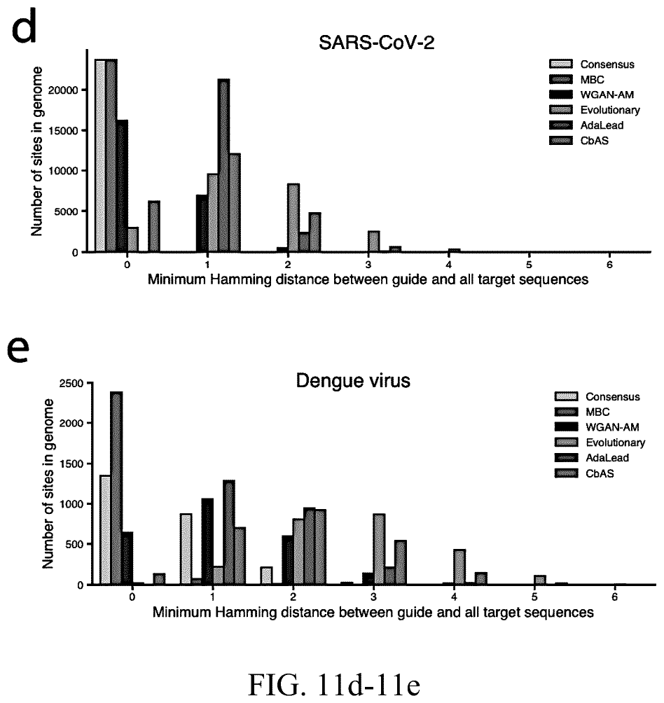

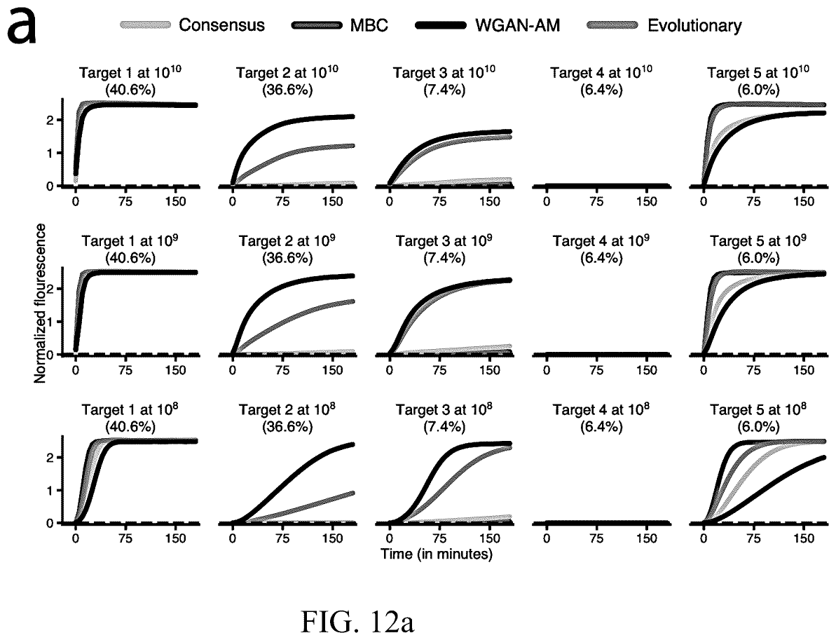

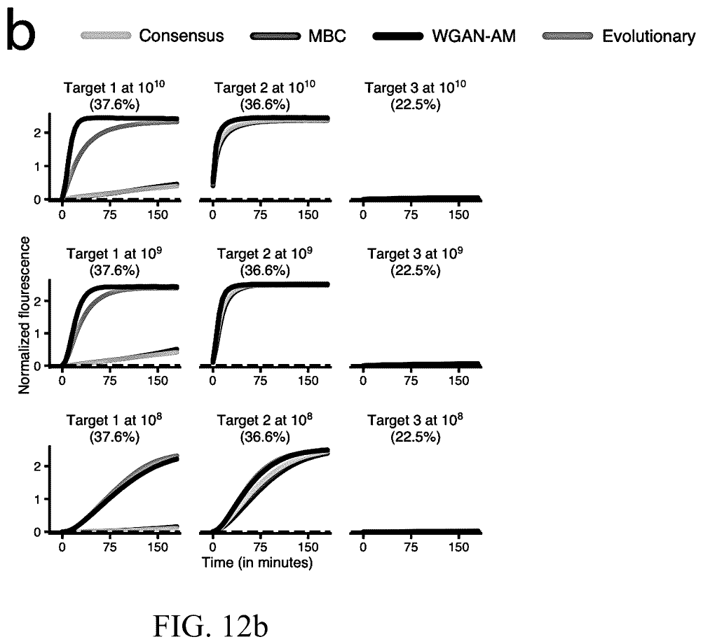

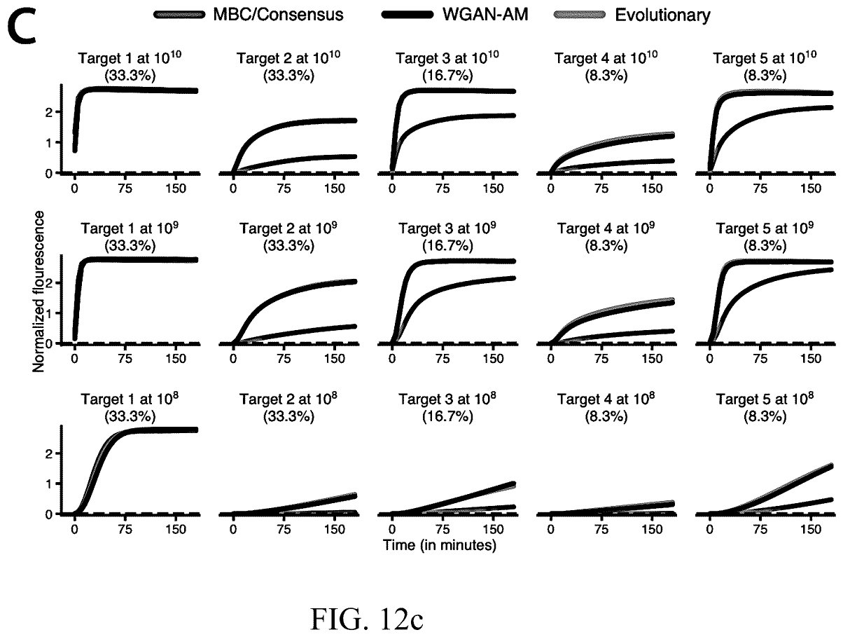

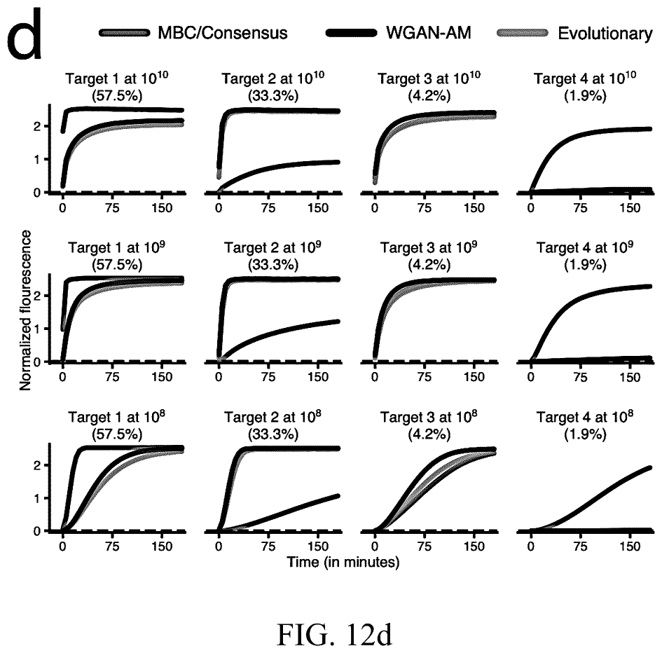

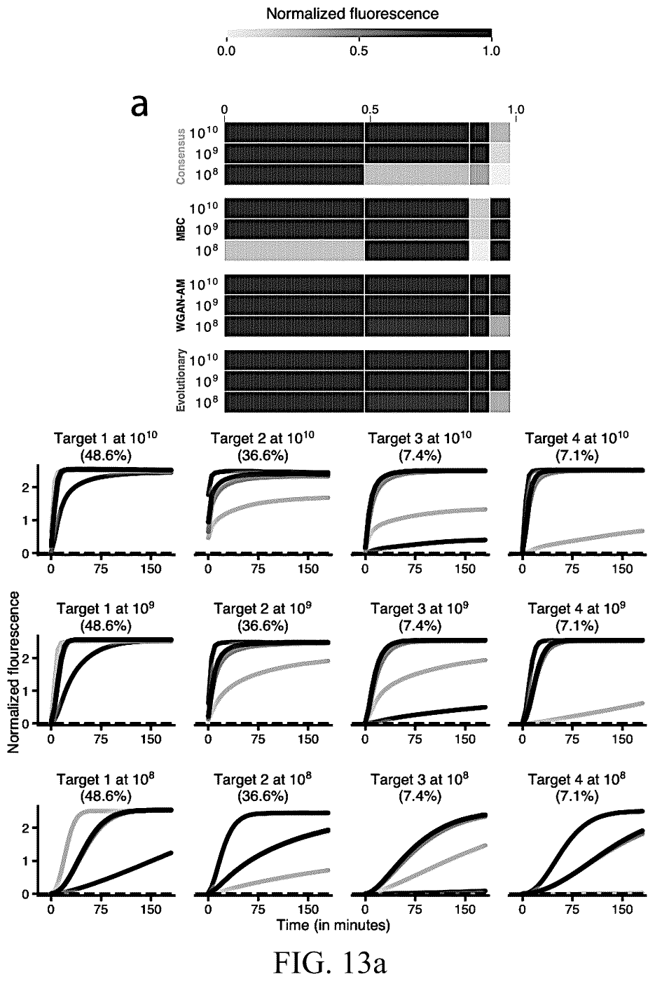

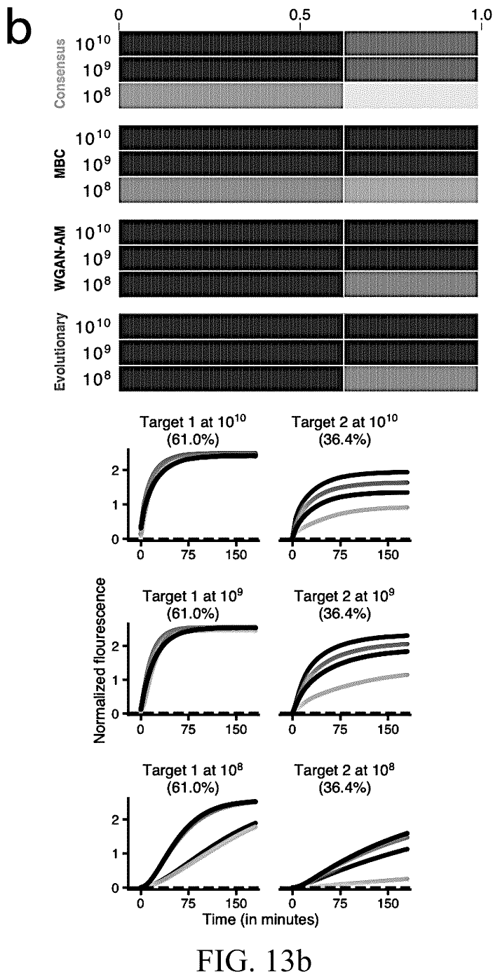

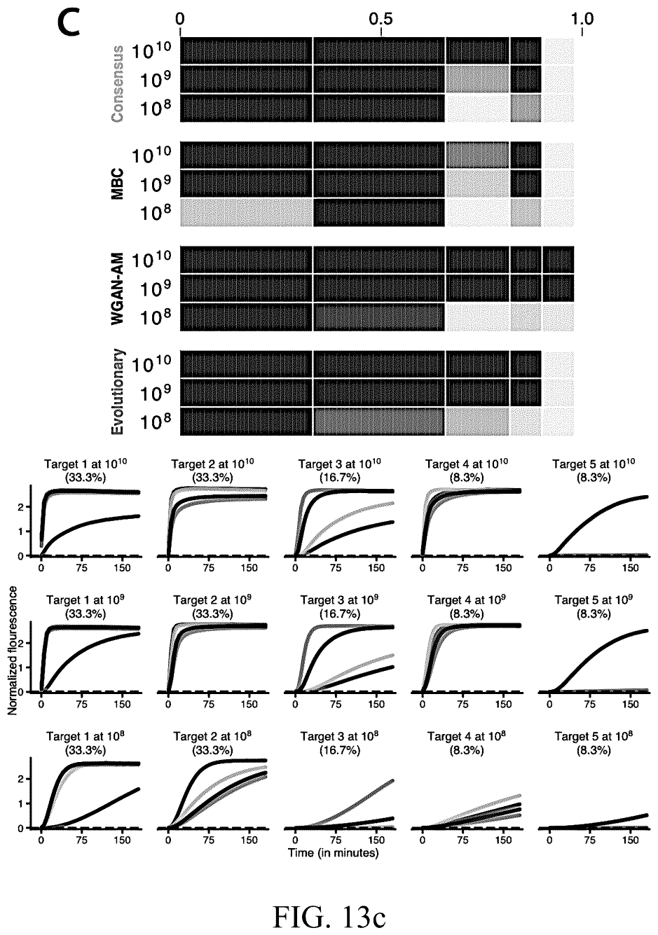

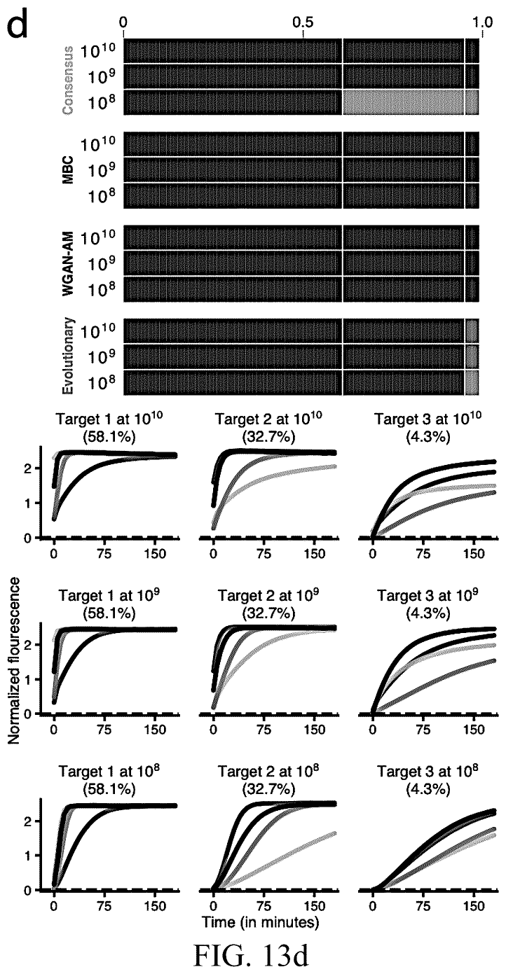

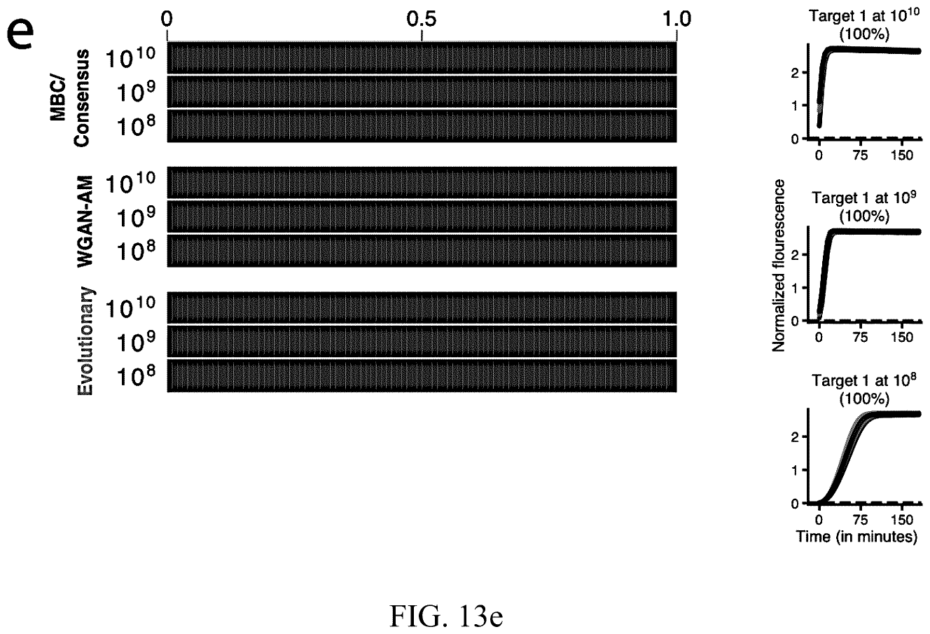

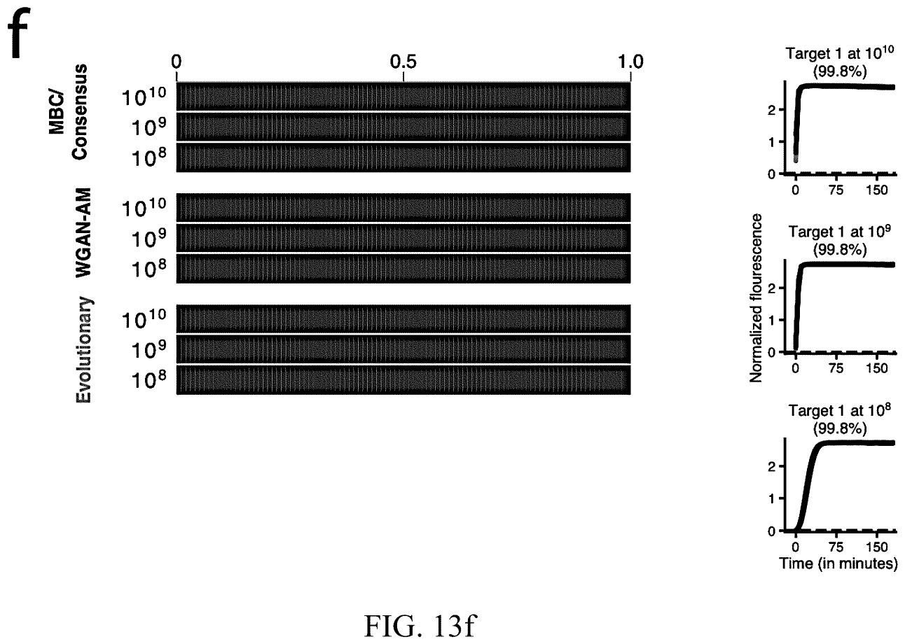

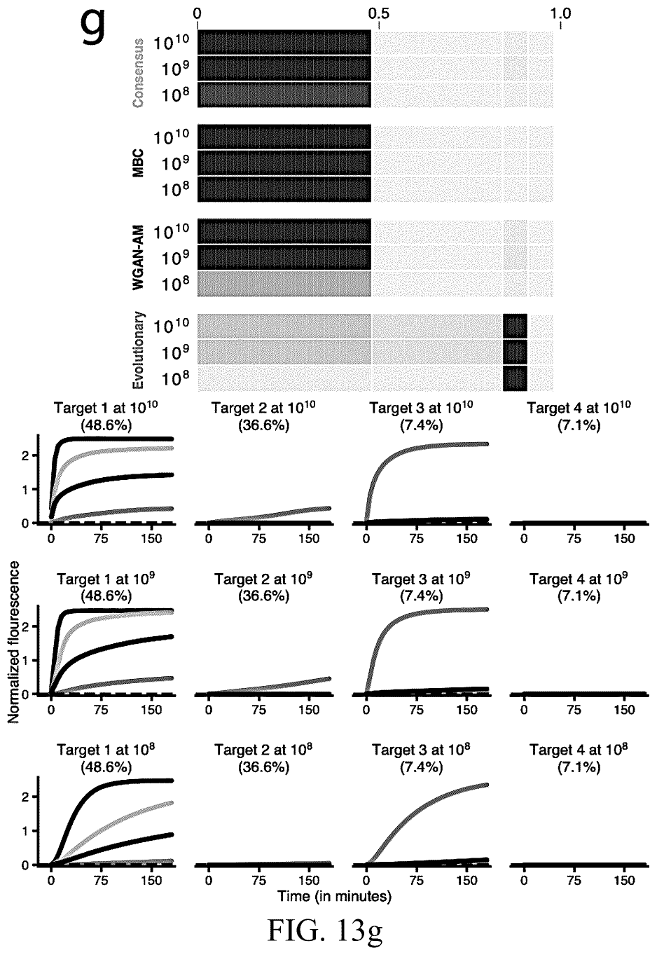

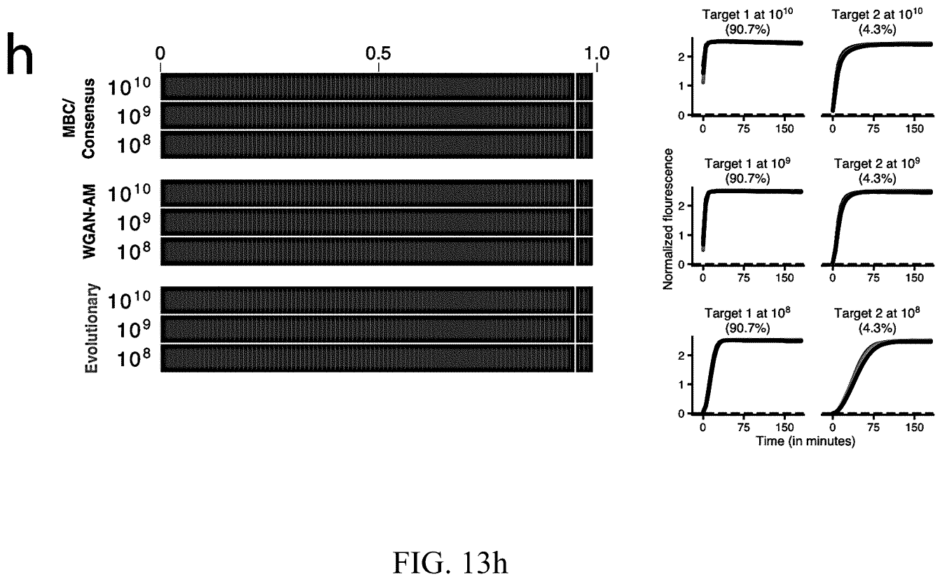

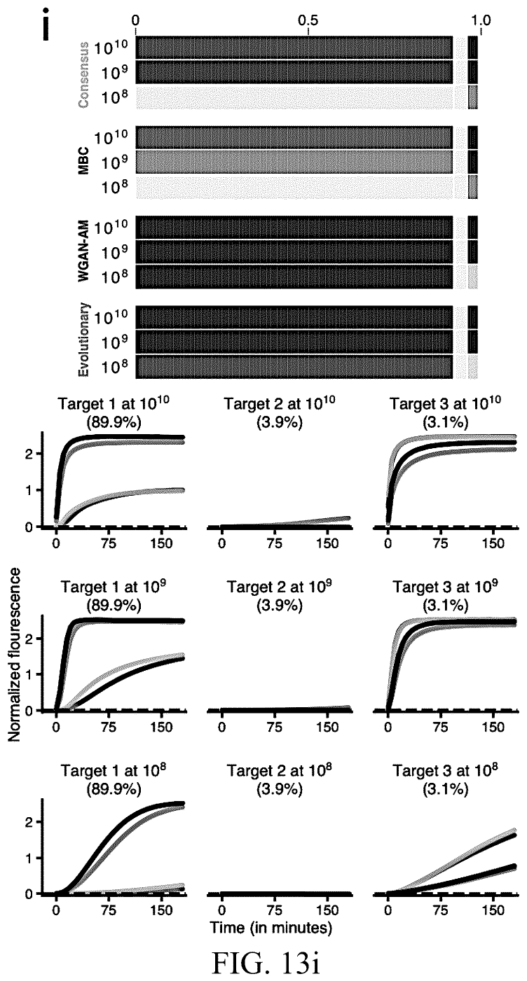

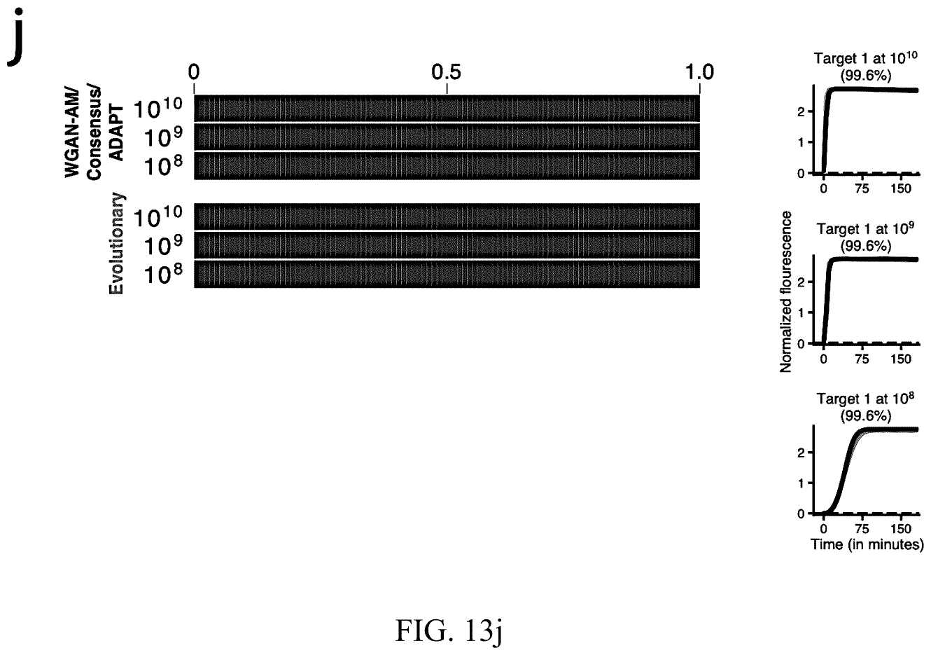

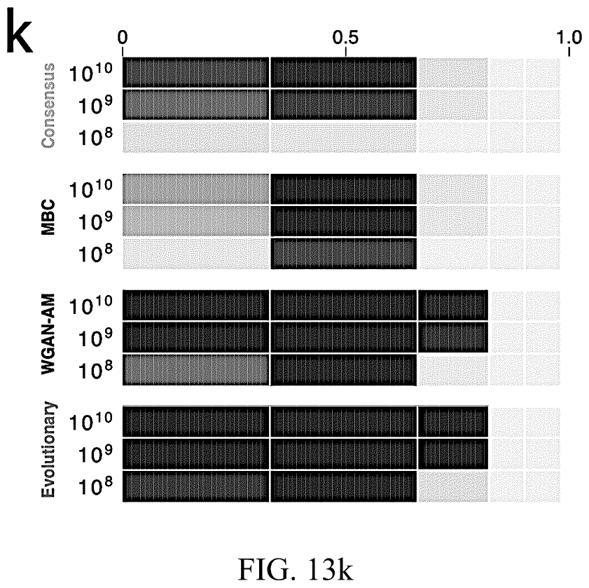

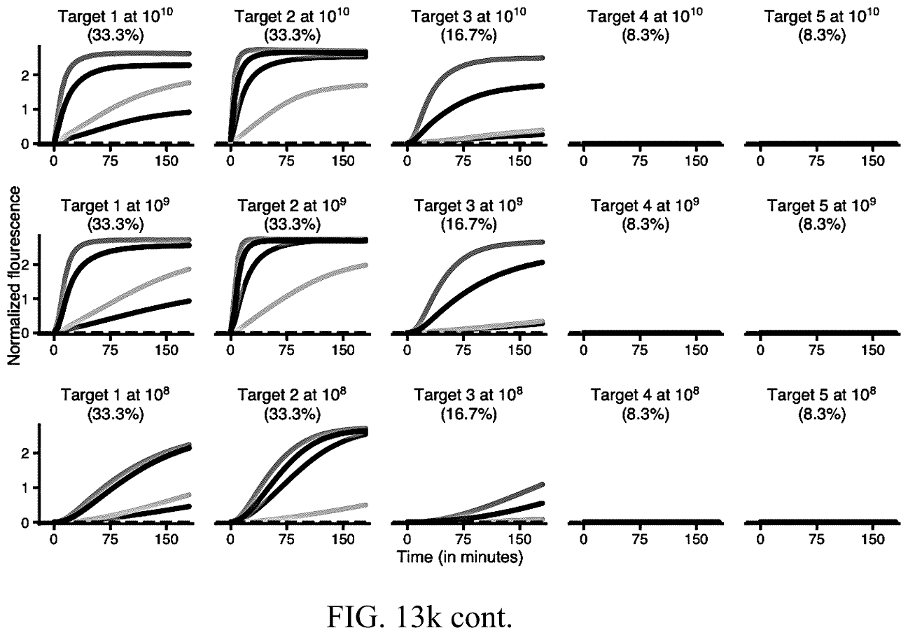

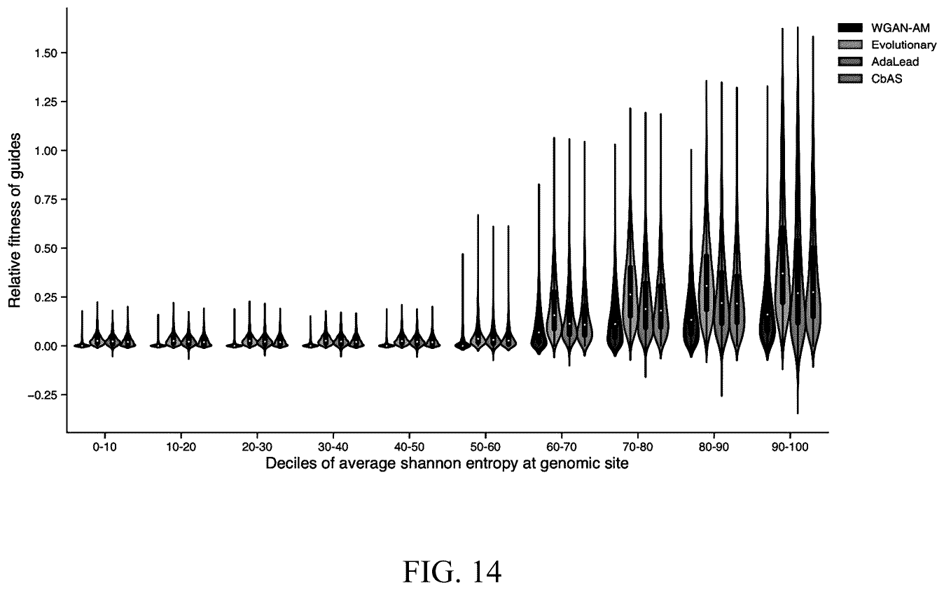

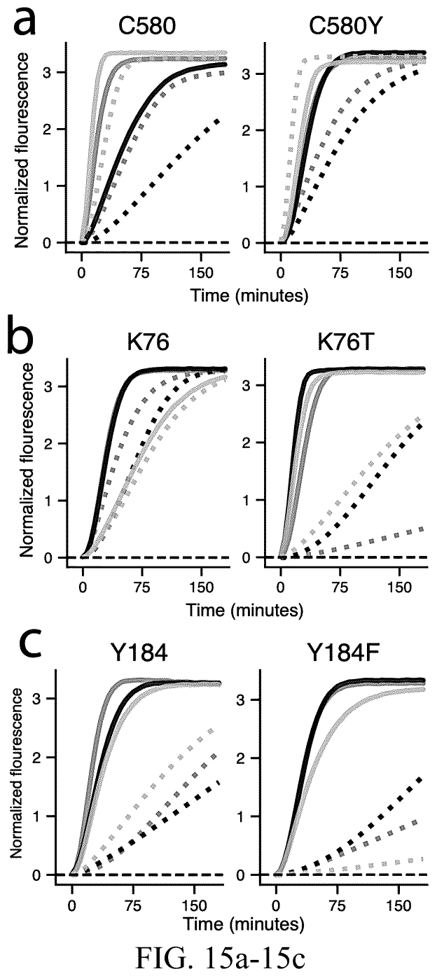

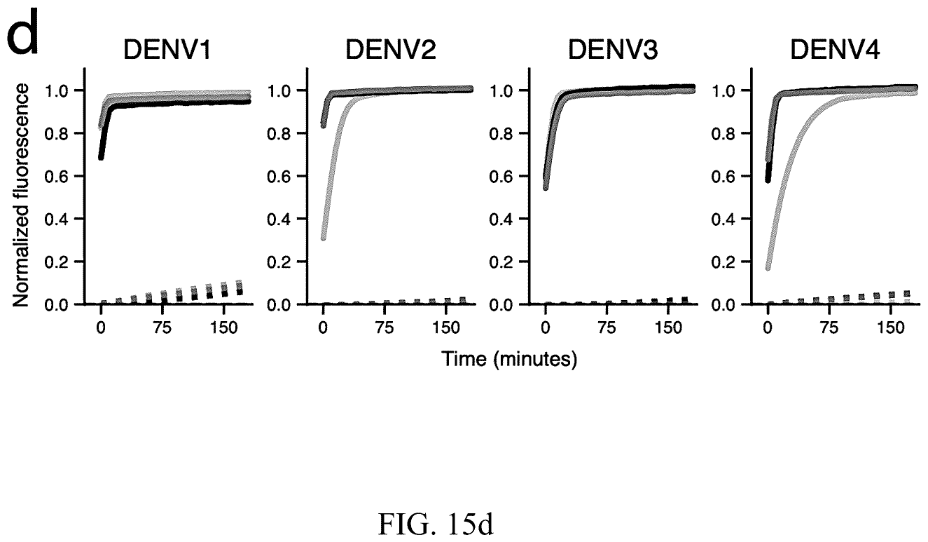

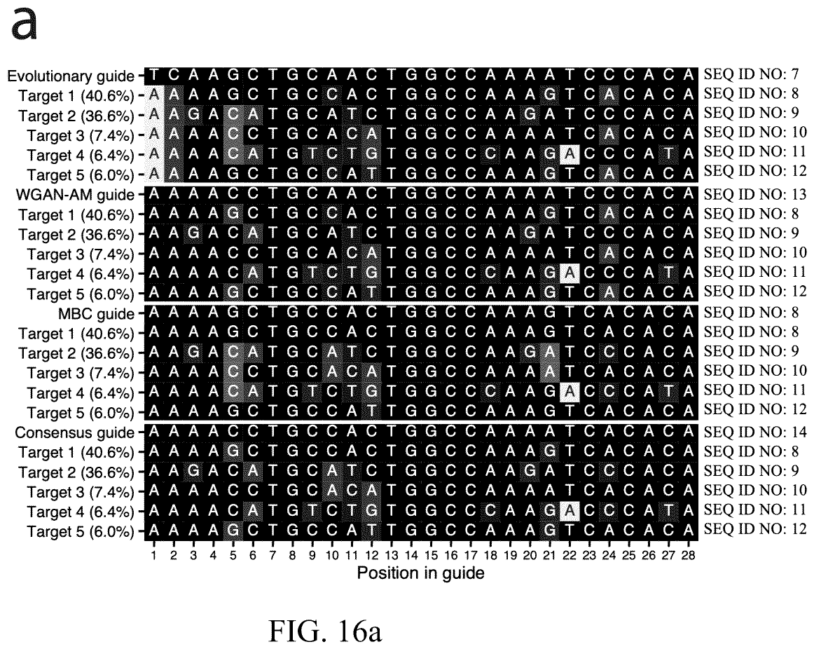

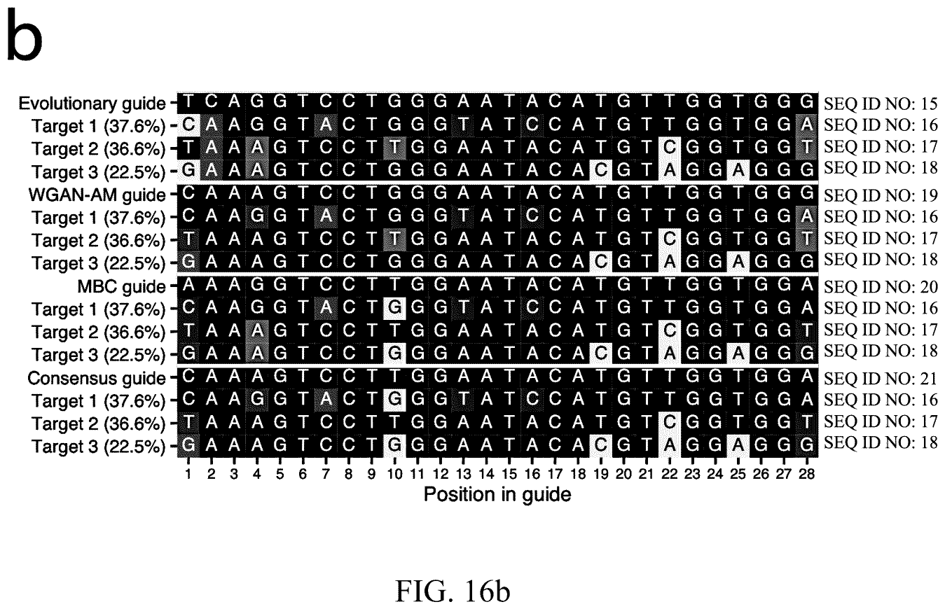

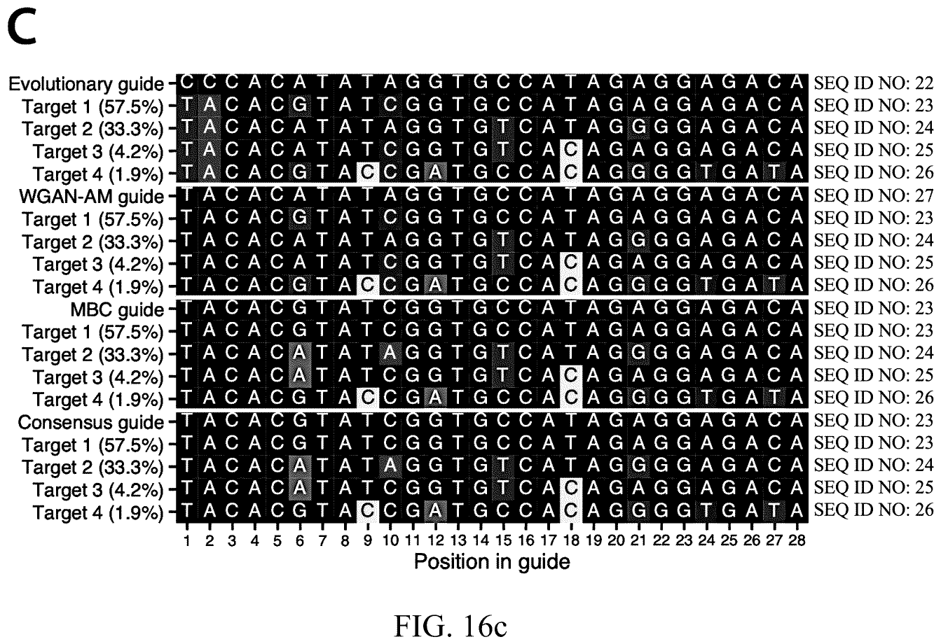

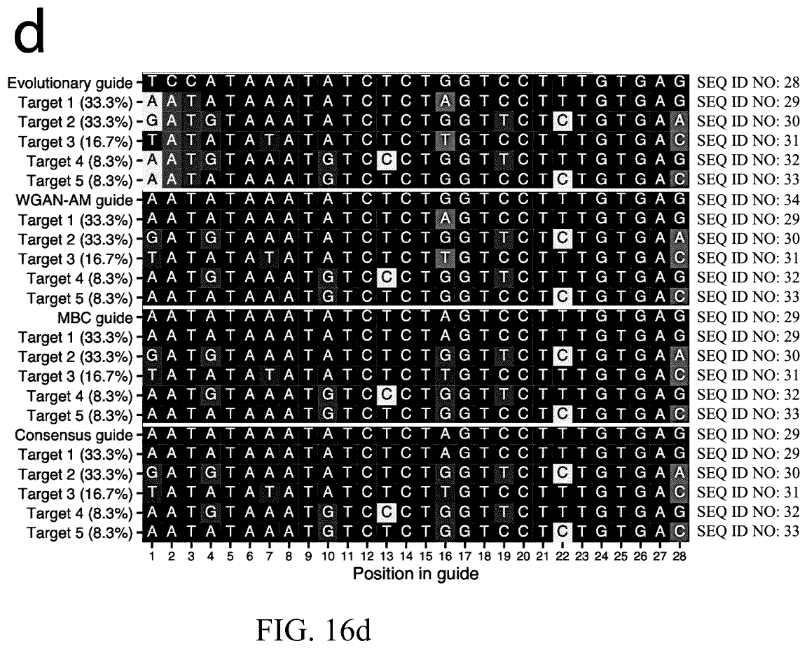

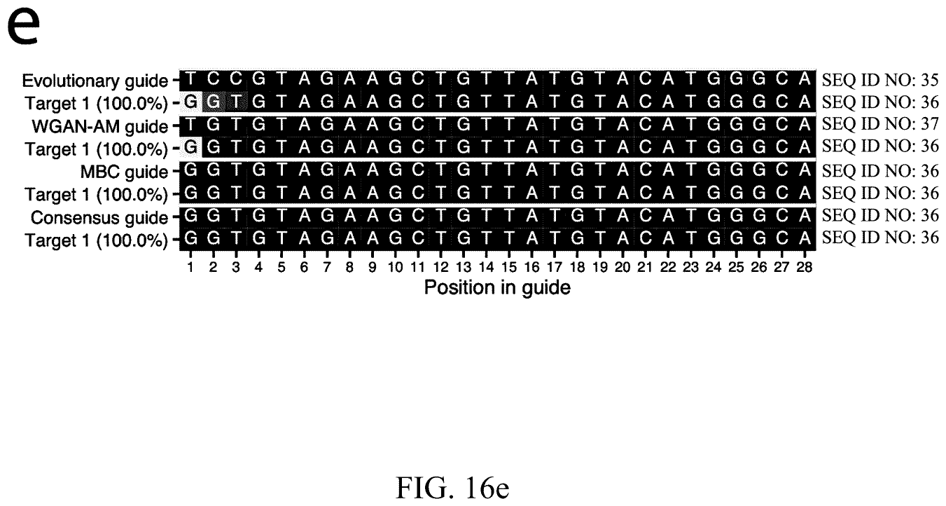

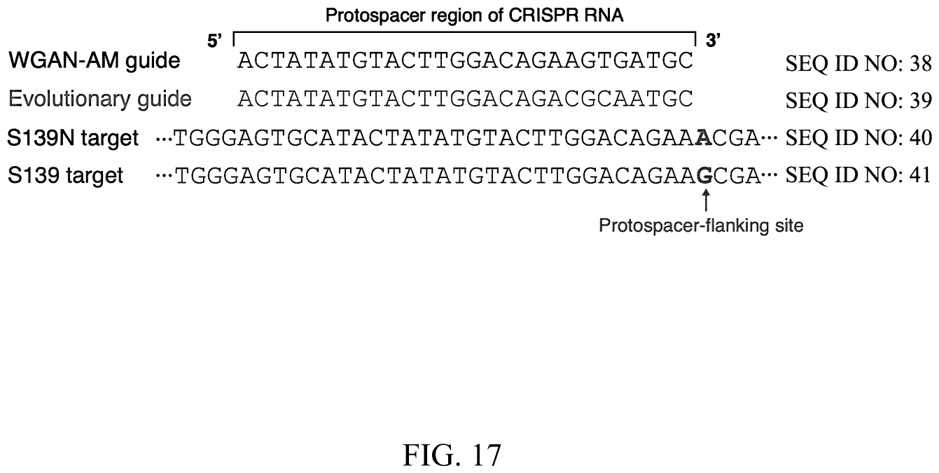

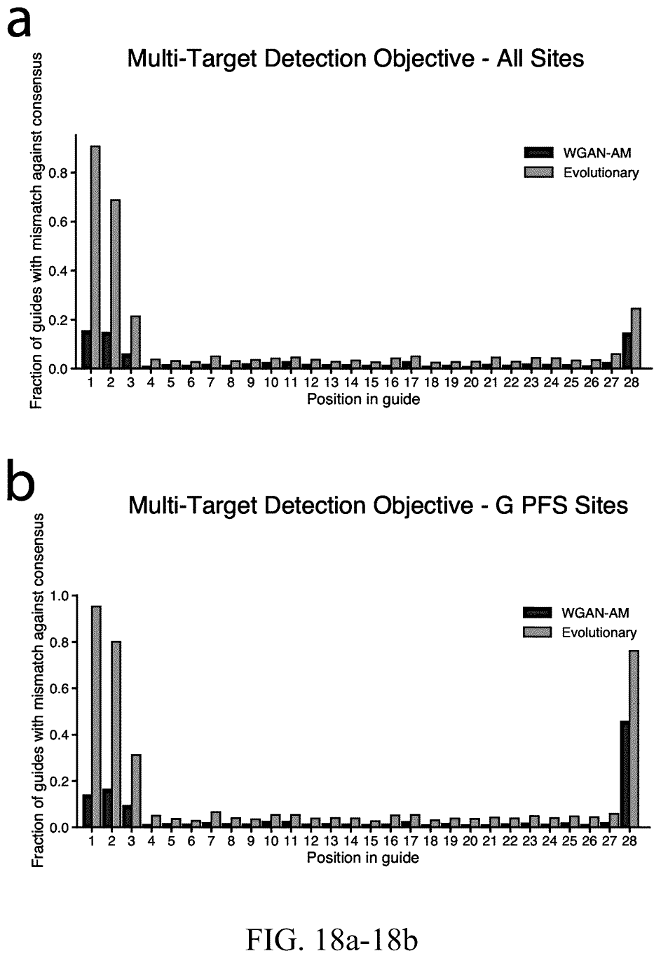

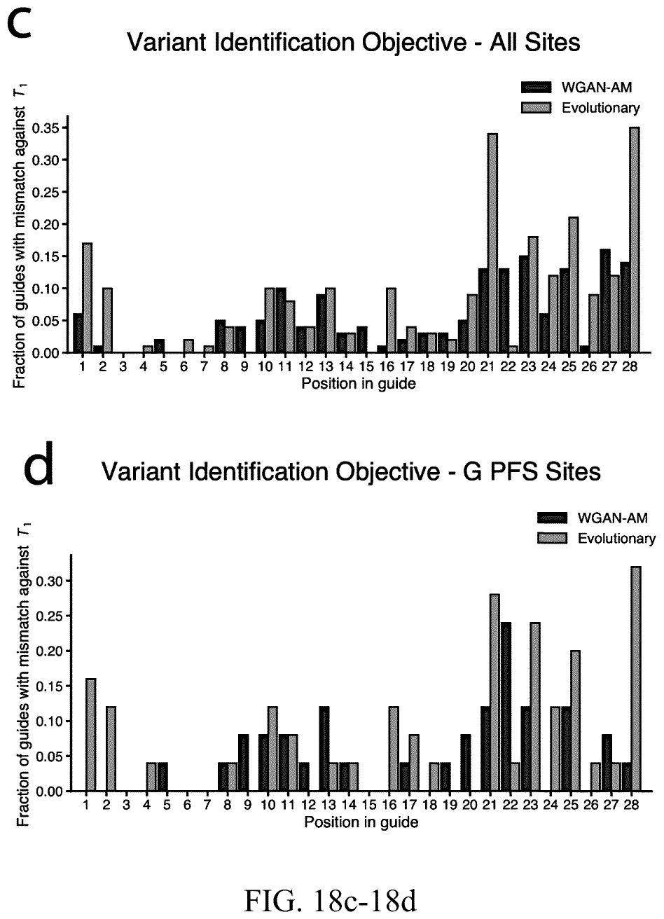

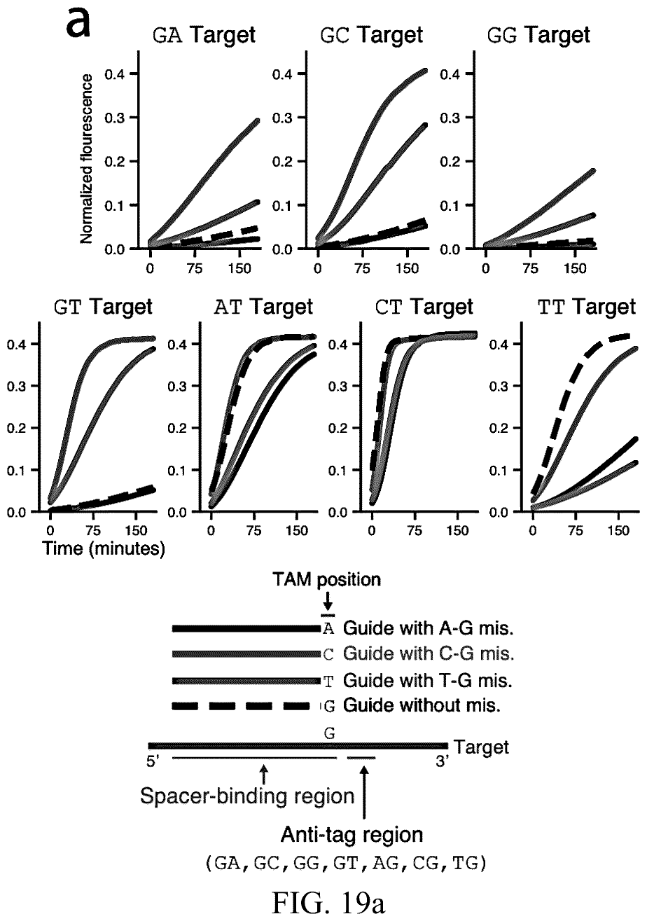

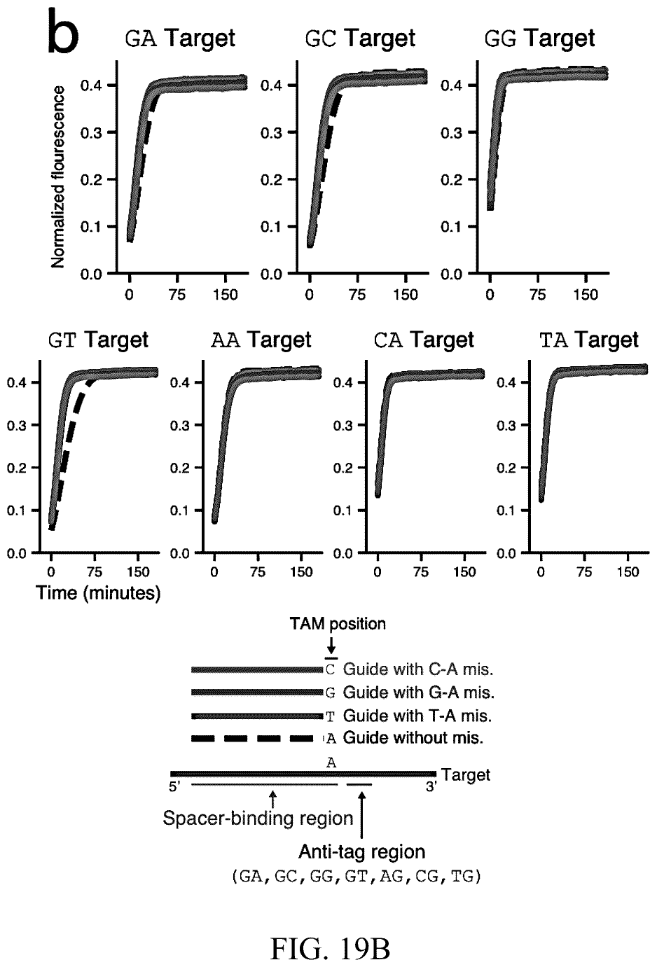

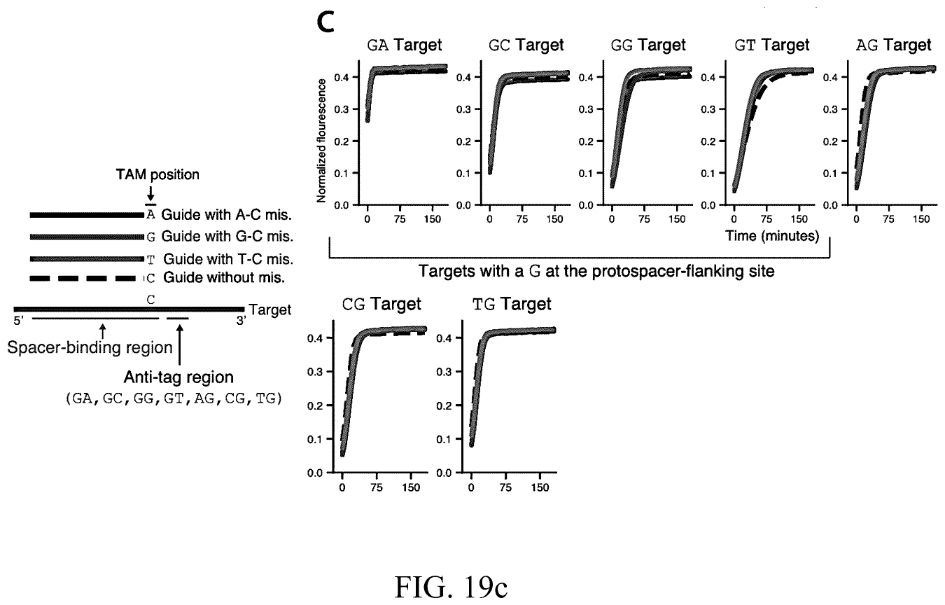

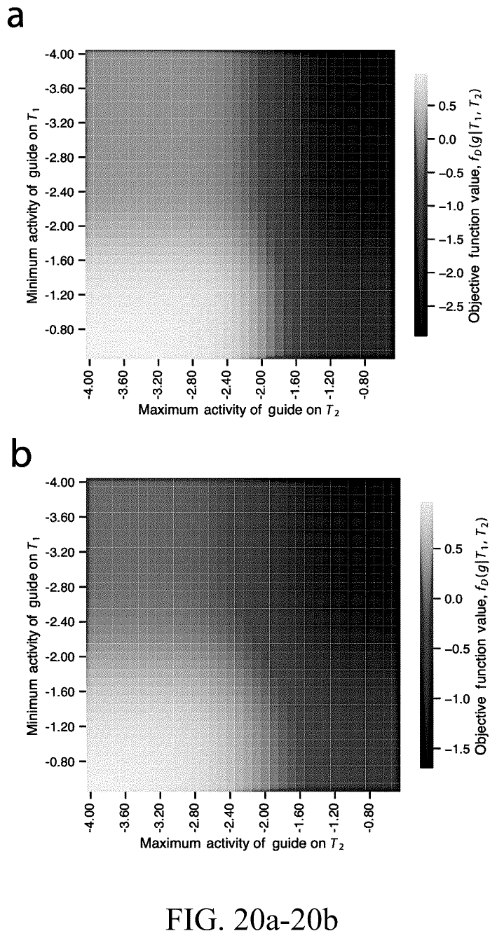

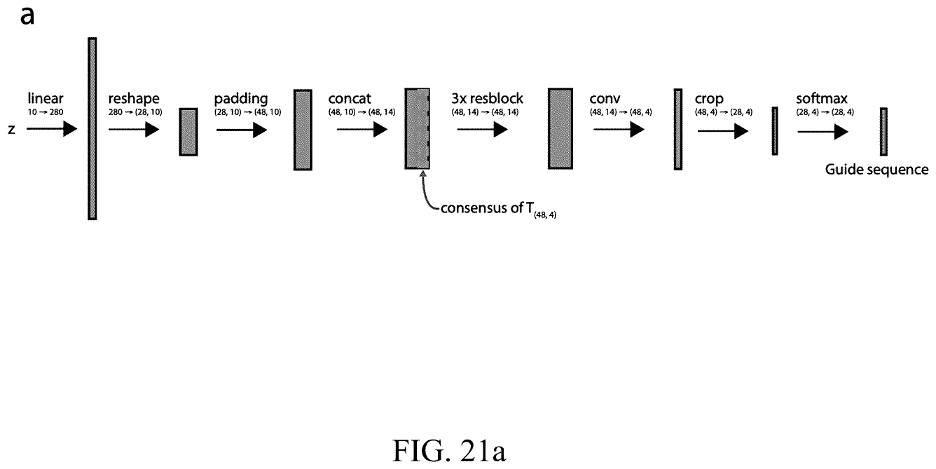

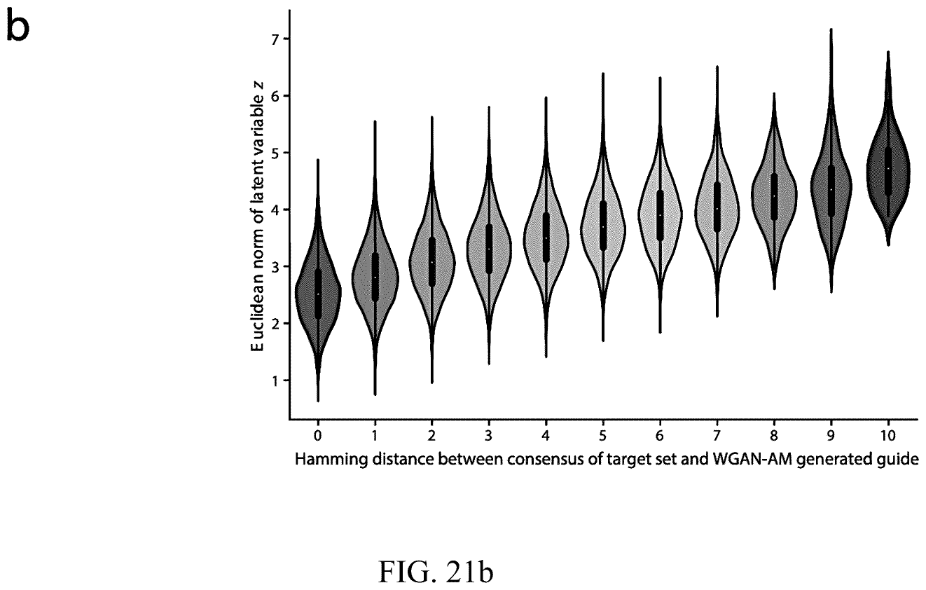

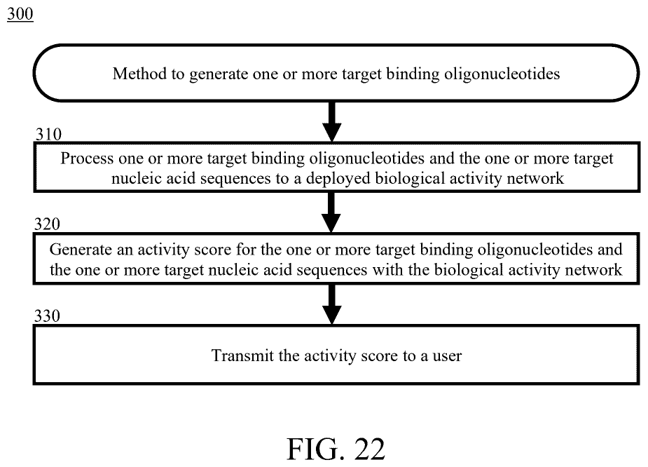

Applicants computationally evaluated the multi-target detection performance of the different design methods across these five pathogens using the same predictive model of guide activity (Methods). BADGERS-designed guides were predicted to detect more diversity than guides designed by baseline methods across nearly all genomic regions of all five pathogens. In several regions within dengue, Lassa, and influenza A viruses, the BADGERS-designed guides were predicted to detect 10-20% more sequence variation (FIG. 5a and FIG. 9). The sequence and nucleotide composition of a viral pathogen vary across different genomic regions and influence the activity of diagnostic guides; thus, BADGERS-designed guides may achieve a coverage improvement in some regions but not others. The BADGERS-designed guides also achieved a higher mean fitness genome-wide (FIG. 5b and FIG. 10a). At most sites, the guides generated by the algorithms in BADGERS are several mismatches away from any sequenced genomes, suggesting that the WGAN-AM and evolutionary methods' ability to search fitness landscapes and generate artificial sequences contributes to their superior performance (FIG. 5c and FIG. 11). For example, in Lassa virus segment S, the WGAN-AM and evolutionary algorithms designed guides that were distinct from any observed sequence at ˜92% and ˜99.9% of genomic sites, respectively.

Computational predictions are inherently limited, so Applicants set out to experimentally benchmark the multi-target detection performance of the BADGERS-designed guides. Applicants used a random sampling strategy (Methods) to select three representative genomic sites from each of the five viruses Applicants considered (15 sites in total) and employed both the algorithms in BADGERS and baseline methods to design guides for these sites. Applicants tested these guides against multiple synthetic targets that represent the genomic diversity of each virus (Methods), measuring the fluorescence readout over time. Cas13a is activated through guide-mediated recognition of a target; once active, it engages in collateral cleavage of a fluorescent reporter to generate the fluorescent readout, with greater fluorescent signal indicating stronger guide-target affinity at a fixed target concentration. All guides were tested against the target concentrations 1010 copies/μL, 109 copies/μL, and 108 copies/μL, which represent typical post-amplification concentrations of viral pathogens^{6, 25, 26}.

For all but one of the 15 tested sites, the BADGERS-designed guides achieved higher activity across sequence diversity than the baseline guides (FIG. 5d-g, FIG. 12, and FIG. 13) and often enabled a lower limit of detection (LoD) as well (FIG. 5h and FIG. 12). The algorithms in BADGERS provided the greatest advantage in highly diverse viruses, e.g., Lassa virus; this advantage is less pronounced at more conserved sites from less diverse viruses, e.g., SARS-CoV-2 (FIG. 9 and FIG. 14).

Applicants next applied the algorithms in BADGERS to the second diagnostic objective: to design guides that optimally identify specific SNPs or distinguish between pathogen lineages, i.e., variant identification (FIG. 4b). This objective can be conceptualized as designing a guide that achieves high activity on an on-target sequence and minimal activity on an off-target sequence; the fitness function is approximately the difference between these activities (FIG. 5a, Methods).

To computationally evaluate the performance of BADGERS's generative design approach designing guides that identify SNPs, Applicants considered a set of 100 pairs of randomly generated on-target and off-target sequences, each differing by one nucleotide (Methods). Applicants applied the algorithms in BADGERS to these target sets and also designed guides using the canonical strategy 17 for this problem: this strategy places the SNP at position 26 of the guide and introduces a synthetic mismatch, at position 24 of the guide, to both the on- and off-target sequences (Methods). MBC's design approach cannot create guides that can differentiate between lineages down to the single nucleotide level, so it could not be applied to this task. In silico, the BADGERS-designed guides exhibited similar predicted on-target activity to those designed by this synthetic mismatch method but achieved lower off-target activity (FIG. 5b-d).

Applicants benchmarked the design algorithms' variant identification performance experimentally, first by designing guides for six clinically relevant SNPs and comparing them to the baseline canonical synthetic mismatch approach. The six SNPs include four associated with antimicrobial resistance in Plasmodium falciparum (Pfcrt K76T, Pfdhps A437G, Pfk13 C580Y, Pfmdr1 Y184F), one associated with microcephaly in Zika virus (PrM S139N), and one associated with immune evasion in SARS-CoV-2's Spike protein (K417N/K417T)²⁷⁻³³. Experimentally, across nearly all of these tasks, the BADGERS-designed guides exhibited lower off-target activities while maintaining similar on-target activities to baseline guides (FIG. 5e-f and FIG. 15a-c). Several baseline guides (including those for the S139, A437G, and K417T targets) had an off-target signal nearly identical to the on-target signal throughout the reaction, which complicates SNP identification; in contrast, the BADGERS-designed guides achieved an on-target signal 2-3 times above the off-target signal (FIG. 5e-f).

Applicants further benchmarked the design algorithms' variant discrimination performance experimentally for designing guides that differentiate viral lineages, focusing on dengue virus (DENV) serotypes 1-4, and seven key SARS-CoV-2 lineages (Methods). In the DENV task, one serotype is the on-target set (e.g., DENV-1) and the other three serotypes comprise the off-target set (e.g., DENV-2, 3, 4). Both the BADGERS-designed guides and baseline guides differentiated the DENV subtypes with high specificity at a target concentration of 108 copies/μL, likely because there are DENV genomic regions with sufficient dissimilarity across serotypes (FIG. 15d). However, when tested at a lower target concentration of 106 copies/μL, the baseline guides exhibited low on-target fluorescence for serotypes 2 and 4, while the BADGERS-designed guides achieved, as desired, a high on-target fluorescence for those serotypes (FIG. 5g). In the SARS-CoV-2 task, Applicants designed guides that differentiate the ancestral Wuhan lineage (i.e., Ref), Alpha, Beta, Delta, Gamma, Omicron/BA.1, and Omicron/BA.2 (Methods). The BADGERS-designed guides enabled robust lineage discrimination, while the baseline guides for Alpha, Gamma, and Ref had off-target activities that were nearly as high as their on-target activities throughout the reaction (FIG. 5h).

Overall, the WGAN-AM and evolutionary search algorithms performed similarly in the two tasks, despite employing distinct techniques and creating different guides. On average, the evolutionary algorithm designed guides that are more distinct from observed sequences than the WGAN-AM's guides (FIGS. 5c, 6d, and FIG. 11). Fundamentally, the WGAN-AM and evolutionary methods represent two different classes of search algorithms: the WGAN-AM samples sequences from a generative network, while the evolutionary algorithm simulates natural selection. To explore the utility of alternative search algorithms, Applicants evaluated the performance of another method in each class. Specifically, Applicants performed in silico benchmarking of CbAS34, which employs a generative network, and AdaLead³⁵, which employs an evolutionary-inspired approach (Methods). Most importantly, all four search algorithms provide a substantial improvement over the baseline approaches. Different search algorithms do exhibit subtle differences in performance depending on the specific task; for example, the WGAN-AM designed guides achieved a lower mean fitness on the multi-target detection objective than guides designed by the other three search algorithms, although this was not the case on the variant identification objective (FIG. 10).

Next, Applicants sought to understand if the algorithms in BADGERS leverage known rules governing Cas13a activity to enhance guide performance. Remarkably, Applicants found that the generative design algorithms implicitly learn Cas13a's protospacer and mismatch preferences, without having them explicitly encoded, and rationally apply this biological understanding among the two design objectives to generate optimally fit guides. For example, guide-target mismatches in close proximity to one another are known to decrease enzymatic activity³⁶⁻³⁸. When applied to the first objective of multi-target detection, the BADGERS-designed artificial guides reduced the number of adjacent guide-target mismatches at mismatch-sensitive positions relative to baseline guides, enabling superior sensitivity (FIG. 16). In contrast, when applied to the variant identification objective, the algorithms in BADGERS introduced artificial mismatches within 4 nt of the SNP for the majority of target sets (WGAN-AM, 61%; evolutionary, 81%), reducing off-target activity (FIG. 5c). Additionally, for nearly 75% of simulated targets, the algorithms in BADGERS further optimized guide specificity by placing the SNP between positions 18 to 23, a region where mismatches are known6 to be highly deleterious to LwaCas13a activity (FIG. 5d).

Furthermore, Applicants found that the algorithms in BADGERS take advantage of another biological property of Cas13a to design guides optimally suited for both objectives. Previous work has found that Cas13a guides exhibit reduced activity when targeting a genomic site with complementarity to Cas13a's tag sequence, particularly targets with a G nucleotide at the position immediately 3′ to the protospacer (the protospacer-flanking site, or PFS)^{38, 39}. In the variant identification objective, when the targeted SNP is a mutation from a non-G to a G nucleotide (as it is in the experimentally-tested S139N and C580Y targets), the algorithms in BADGERS positioned the G at the PFS to achieve minimal off-target activity (schematized in FIG. 17; experimental results in FIG. 5e and FIG. 15a). However, in the multi-target detection objective, the algorithms in BADGERS exhibited an unexpected design decision. For genomic sites with a G at the PFS, the algorithms often introduced a mismatch directly adjacent to the tag sequence at position 28 in the protospacer (the same as position 1 in the spacer), which Applicants term the tag-adjacent mismatch, or TAM. Intriguingly, among the genomic sites with a G at the PFS, the TAM was present in 45.7% of guides designed by the WGAN-AM algorithm and 76.2% of guides designed by the evolutionary algorithm (FIG. 18).

Applicants hypothesized that the TAM could represent a novel design strategy, discovered by the algorithms in BADGERS, that enables improved guide activity on targets with a G at the PFS, so Applicants explored the TAM's significance further. Mechanistically, the TAM may disrupt base pairing between the G at the PFS of the target and the C in the CRISPR RNA tag sequence, promoting the separation of the Cas13-target complex and thereby increasing collateral cleavage activity and the resulting fluorescent signal. To test this hypothesis, Applicants designed a library of guides against targets having unfavorable nucleotides (GN) and favorable nucleotides (AN, TN, or CN) for the first two positions of the anti-tag region; the first nucleotide of the anti-tag region is the PFS (Methods; FIG. 5i). Guides with a TAM targeting a sequence with a G at the PFS exhibited substantially greater activity than guides without this TAM (FIG. 5i and FIG. 19). Furthermore, the TAM conferred the greatest benefit when the targeted sequence was GT in the anti-tag region. This is mechanistically explainable: the first two nucleotides of Cas13a's tag sequence complement GT, so the TAM's disruption of this double base pairing may enhance activity more than for targets with a non-GT allele (FIG. 5i and FIG. 19). As expected, introducing a TAM to guides targeting a sequence with a non-G at the PFS did not increase activity. To Applicants' knowledge, this design principle of introducing a mismatch in the spacer to rescue the activity of guides with extended tag: anti-tag complementarity has not been described before, suggesting that the algorithms in BADGERS are capable of elucidating new design principles.

In this work, Applicants demonstrated that generative design algorithms can yield maximally-fit guide sequences for pathogen detection and surveillance, and can illuminate novel guide design rules. These algorithms leveraged a predictive model to explore a sequence landscape, only evaluating ˜4,000 out of ˜10¹⁶ potential guides throughout their search process, yet are capable of designing artificial guides that outperform those derived directly from nature (Supplementary Note 1). Crucially, this approach is not trained on nor restricted to a specific design task, but rather can generate guides for any conditioned target set. The machine-learned models of guide activity that steer the optimization process enable the method to exploit features of Cas13a's underlying biology (e.g., anti-tag and mismatch preferences) to generate guides that optimize diagnostic performance. During the early 2022 surge in COVID-19 cases, Applicants applied a prototype version of the method to design guides that distinguish SARS-CoV-2 variants, demonstrating that BADGERS-designed panels achieve high concordance with next-generation sequencing in a clinical context⁴⁰. Furthermore, these methods can reveal new, interpretable guide design rules for CRISPR-based technologies. The TAM proposed by the generative design algorithms may enable Cas13a-based technologies to more efficiently target a broader range of sequences and improve the understanding of the enzyme's nuclease activation mechanisms.

While false positive sequences, which score highly computationally yet are non-functional experimentally, are a challenge in generative sequence design, none of the BADGERS-designed guide sequences fell into this category. This may be partly due to the nature of the tasks; the focus on nucleotide base pairing entails substantially less structural complexity and a smaller sequence space relative to other tasks (e.g., protein design). Nevertheless, the design algorithms that Applicants evaluated still rely on predictive models of guide activity, which may return inaccurate predictions for guide-target pairs outside of the training domain. Certain design approaches—such as the WGAN-AM, which was trained to generate active guide-target pairs, or CbAS, which considers uncertainty in activity predictions—may be less susceptible to generating out-of-domain sequences than methods like the evolutionary algorithm that are less constrained in their search. Additionally, these methods may not perform as well when applied to pathogens whose sequence composition is highly dissimilar from the viral sequences that comprised the predictive model's training dataset. Also, because different orthologs of Cas13 are known to have varying sequence preferences, the design algorithms that Applicants evaluated may not be reliable in designing guides for non-Cas13a enzymes. Furthermore, although these algorithms do enhance diagnostic sensitivity, many regions of viral genomes are hypervariable and, in some cases, even the guides designed by these methods do not detect the full extent of sequence diversity (e.g., dengue virus and enterovirus B; FIG. 5e-f).

In the realm of pathogen diagnostics, to Applicants' knowledge, all current design methods for nucleic acid technologies derive oligonucleotide sequences directly from pathogen genomes¹⁻⁶. The results suggests that generative sequence design algorithms offer substantial benefits relative to approaches, like the one in ADAPT, that are not geared toward designing artificial guides. Namely, the algorithms in BADGERS created guides that achieved superior performance to MBC (the technique employed by ADAPT) on the multi-target detection objective and enabled the discrimination of lineages down to single nucleotide resolution, a task ADAPT cannot perform. To achieve this, BADGERS's methods engineered guide sequences by leveraging rational design principles (e.g., G-U base pairing and nucleotide and position-specific effects of mismatches) and discovered new strategies for guide design (including the terminal adjacent mismatch).

Though Applicants focused on Cas13 guide RNAs in this work, artificial sequences stand to optimize the design of diagnostic sequences beyond CRISPR guide RNAs, including the primers and fluorescent probes employed in PCR, loop-mediated isothermal amplification (LAMP), and recombinase polymerase amplification (RPA). While the specific search algorithms employed in this work to generate artificial sequences achieved strong performance, the field of biological sequence design is rapidly advancing with the recent application of other classes of design methods (e.g., autoregressive and diffusion-based generative methods), and future work should investigate how these approaches may further benefit diagnostic design. Beyond diagnostics, generative design methods could design rationally mismatched guide RNAs for base editors to minimize bystander editing rates¹⁶. For certain CRISPR screens, where the objective is to introduce maximal sequence diversity into protein-coding genes, these methods could design guides that maximize the number of distinct edits made at the targeted sites⁴¹. Furthermore, while high-fidelity variants of CRISPR enzymes have lowered off-target editing rates⁴², this class of algorithms could optimize guide RNA sequences to minimize these off-target effects further. Recent work has also highlighted the importance of accounting for human genetic diversity when designing CRISPR therapeutics^{42, 43}, and generative design approaches are poised to generate guide sequences that optimally account for this diversity.

Generative methods are transforming the field of biological sequence design, with recent work focusing on generating long protein and DNA sequences^{10, 12, 44}. Here, Applicants showed that algorithms which explore sequence landscapes to design artificial sequences can not only optimize shorter RNA sequences, such as CRISPR RNAs, but can simultaneously elucidate new mechanistic design rules and serve needs in pathogen surveillance.

### Methods

The model-directed exploration algorithms developed in this work pair (1) a predictive model of diagnostic guide activity with (2) exploration algorithms that search through a landscape of guide sequences. Together, they design guides that optimize a well-defined objective function.

A guide sequence is a ˜20 to 30 nucleotide (nt) nucleic acid sequence that binds to a target. In The applications, the guide is the 28 nt CRISPR RNA (crRNA) spacer sequence for use with CRISPR-Cas13a-based detection. It allows detection of a target by leading to a readout when the target is present.

### Predictive Model of Diagnostic Guide Activity

Applicants previously developed the predictive models used in this work and they are described in detail in ref. 6, hereby incorporated by reference. The input into these models is a 28-nt guide sequence and a 48-nt target sequence (having 10 nt of context on each site of the guide's binding region). The output of the classification model C(g|t)∈[0,1] can be interpreted as the probability that a given guide-target pair is active. The output of the regression model R(g|t)∈[−4,0] is the predicted activity of an active guide-target pair. Higher values of C(g|t) may suggest that a guide is more likely to bind to a given target, and higher values of R(g|t) may suggest that, after binding, a guide's signal on a given target will grow faster.

In this work, Applicants combined the classifier and regression model's predictions to define a weighted activity metric, A, which follows from the law of total probability:

$$A ( g \vert t ) = P ( g is active on t ) \times ( activity of active g on t ) + ( 1 - P ( g is active on t ) ) \times ( activity of inactive g on t )$$

In this case, C(g|t)=P (g is active on t), R(g|t)=activity of active g on t, and −4=activity of inactive g on t because guides that are inactive have a near-zero signal, which is represented in the dataset by an activity of −4, the lower bound of measured signal.

Thus, for a given guide-target pair, Applicants computed A(g|t) as follows:

$$A ( g \vert t ) = C ( g \vert t ) \times R ( g \vert t ) + ( 1 - C ( g \vert t ) ) \times - 4$$

### Objective Functions for Diagnostic and Surveillance Applications

Model-directed exploration algorithms can design a guide sequence that optimizes a well-defined objective function across a genome's sequence variation.

Applicants focused on designing guides that optimize two specific objective functions. These objectives have applications in diagnostic testing and pathogen genomic surveillance.

### Multi-Target Detection

In the first application (“multi-target detection”), the goal is to design guides that are maximally active across genome sequence diversity. As such, the objective function for this application is:

$${f}_{M} ( g \vert t ) = {E}_{t ∈ T} [ A ( g \vert T ) ] = {\sum }_{t ∈ T} {w}_{t} \cdot A ( g \vert T )$$

where T is the set of genome sequence targets and t∈T is a single target.

Genome sequence diversity varies spatially and temporally, so the wₜ represents a prior probability of encountering a target t∈T. Applicants set a uniform prior distribution (assuming that all wₜ are equal to

$$\frac{1}{\vert T \vert} )$$

over the target genome sequences, following the choice in ref. 6, but one could modify wₜ as certain lineages of a pathogen become more or less prevalent in a geographic region of interest or over time.

Additionally, one could also consider a defining more complex objective functions (such as the 10th percentile of the distribution of activity across genomic diversity), although these functions may be non-differentiable and more difficult for certain exploration algorithms to maximize.

### Variant Identification

In the second application (“variant identification”), the goal is to design a diagnostic guide g that can optimally differentiate between closely related sequences, maximizing activity against one set of targeted sequences (on-target) while minimizing activity against a separate but homologous set of non-targeted sequences (off-target). In this notation, T₁ represents the on-target sequences and T₂ represents the off-target sequences.

Initially, Applicants considered a simple objective function: the difference between the expected on-target activity and expected off-target activity, E_{t∈T}_{₁}[A(g|T₁)]−E_{t∈T}_{₂}[A(g|T₂)]. Although many guides designed using this objective had a large difference between the expected on-target activity and expected off-target activity, they were often inactive or only marginally active on the on-target sequences—making these guide designs unusable—because the function does not enforce that the guide's activity on the on-target set of sequences be sufficiently high for detection.

Thus, Applicants defined an objective function for the variant identification application that is maximized only when the designed guide has high activity on the on-target sequences and low activity on the off-target sequences. Applicants based this objective function upon the logistic function so it is differentiable. The objective is defined below:

$${f}_{D} ( g \vert {T}_{1} , {T}_{2} ) = ( \frac{1}{1 + {ae}^{k ( - logsumexp ( - A ( g \vert {T}_{1} ) ) - o )}} - 1 ) + r \cdot ( \frac{- 1}{1 + {ae}^{k ( logsumexp ( A ( g \vert {T}_{2} ) ) - o )}} )$$

where o, a, k, and r are all parameters that modulate the slope and curvature of the objective function. The values of these parameters were determined through a random search procedure described in the hyperparameter search section of the Methods. Applicants use log sumexp(A(g|Tᵢ)) as shorthand for log sumexp({A(g|t) ∀t∈Tᵢ}), where log sumexp(x₁, . . . , xₙ)=log(exp(x₁)+ . . . +exp(xₙ)) is a smooth approximation to the maximum.

The value of f_{D}(g|T₁,T₂) increases as the minimum activity of the guide across the on-target set increases and the maximum activity of the guide across the off-target set decreases. A visualization of this objective function is included in FIG. 20.

### Exploration Algorithms

The above objective functions define the fitness of a guide for two types of detection tasks. The goal is to design maximally-fit guide sequences—those that maximize the value of these objective functions. Applicants developed two algorithms, a Wasserstein generative adversarial network-activation maximization (WGAN-AM) approach and an evolutionary algorithm, that search over a landscape of guide sequences to generate these maximally-fit guides.

Conditional Generative Adversarial Network with Activation Maximization (WGAN-AM)

A generative adversarial network (GAN) is an unsupervised deep learning framework that approximates an underlying data-generating distribution⁴⁵. A well-trained GAN can generate new samples of data (e.g., photographs or genomic sequences) that have properties similar to those of the training set.

Applicants use a Wasserstein GAN (WGAN) inspired by the application in ref. 20, but with one key difference. The WGAN uses a conditional GAN, in particular conditional on a given target set⁴⁶. The generator network, G(z|T), generates a guide sequence for the given target set. This difference allows the method to generalize well across different target sets, which contrasts to using a GAN that has been fit to data derived from a single target set. The WGAN uses a latent variable z as an input, and z can be modulated to explore the sequence space and generate different guide sequences for a given target set.

The generator network of the WGAN proceeds in five stages. First, the input latent vector z is up-sampled to a vector of |z|×guide length. In the implementation, the latent vector is 10-dimensional and the guide has a length of 28, so the resulting up-sampled vector has length 280. Second, this vector is reshaped into a matrix with dimensions guide length×latent channels (28 by 10). Third, this matrix is padded with a 10 by 10 matrix of zeros on each side such that the dimensions of the resulting padded matrix is 48 by 10. Fourth, the 48 by 10 padded matrix is appended to the one-hot-encoded consensus of a 48-nt region of the target, which has dimensions of 48 by 4. The resulting matrix has dimensions 48 by 14. This makes the generator conditional a given set of targets, because a representative sequence (the consensus) is directly concatenated in this operation. Fifth, this matrix is passed through three residual blocks and 4 convolutional filters with stride 1 and width 2 to create a 48 by 4 matrix. Finally, a softmax is applied such that the resulting matrix represents the probability of having a base at each position, and the 10 by 4 context is removed from either side. The resulting 28 by 4 matrix represents the 28 nucleotide guide sequence, which is the guide that Applicants sought to design. FIG. 21a schematizes the architecture of the generative network of the WGAN.

Applicants trained the WGAN on the same set of active guide-target pairs that the previously developed CNN regression model was trained on⁶. These guide-target pairs were experimentally demonstrated to be active via the CARMEN-Cas13 system⁶. Thus, the generator outputs guides with measurable activity against the conditioned target. Applicants trained the WGAN via the WGAN-gradient penalty method in ref. 47. Applicants used a batch size of 32 and trained the critic 5 times for each batch on which the generator was trained. Applicants used the following hyperparameters for the Adam optimizer⁴⁸: α=0.0001, β₁=0.9, β₂=0.999, and ϵ=10-7

The generator network G(z|T) captures the high-level features that make a guide active against a given target set, and is capable of generating an artificial guide sequence given a target set. To generate maximally fit diagnostic guides, Applicants employed a joint activation maximization framework²⁰. In this joint framework, the WGAN's generator, G(z|T), transforms the latent vector z into a candidate guide sequence g, and the objective function, ƒ(g|T), computes the guide's fitness.

To generate optimal guides, Applicants compute the gradient of the guide's fitness with respect to the latent vector z, and use the Adam optimizer⁴⁸ to take steps in the direction of this gradient:

$${z}^{( t + 1 )} = {z}^{( t )} + ( step size ) \cdot {∇}_{zf} ( g \vert T ) ,$$

$$And {∇}_{z} f ( g \vert T ) = {\sum }_{d} \frac{\partial f ( g \vert T )}{\partial {x}_{d}} \cdot \frac{\partial {x}_{d}}{\partial z}$$

and d is one of the 10 dimensions of the latent vector

This procedure allows for a continuous search over the latent space to generate guide sequences that have optimal fitness. To more thoroughly explore the fitness landscape and account for the fact that several local minima may exist in the fitness landscape, Applicants perform multiple searches over the latent space with each search starting at a different random starting point (sampled from a normal distribution) in the latent space. A pseudocode overview of the WGAN-AM algorithm is available in FIG. 7 and a high-level schematic is presented in FIG. 4c.

The WGAN-AM algorithm's generated guide sequences are implicitly constrained. Since the WGAN's generator network approximates the conditional probability distribution P(g|T) of the guide-target pairs in the training set, the guide sequences output by the WGAN's generator network have similar properties (e.g., mismatch positions and mismatch types) to the guide sequences used to train the regression model. Thus, the WGAN-AM algorithm searches regions of the fitness landscape in which the regression model is likely to accurately predict guide activity. Because of the generator network's structure, it is unlikely that the WGAN-AM algorithm will design guides that are so different than the guide sequences in the training set that the regression model's predictions are no longer accurate and the model behaves pathologically. For example, Applicants would not expect the WGAN-AM algorithm to generate guides that have a much higher number of mismatches against their target sets than those in the training data for the predictive models.

### Evolutionary Algorithm

Applicants also tested an unconstrained search algorithm that could reach any region of the fitness landscape—an evolutionary algorithm. Evolutionary algorithms are biologically-inspired optimization algorithms that are widely used in computer science to search across a domain of candidate solutions. They have been applied to design biological sequences that exhibit a desired property, such as peptide sequences that have maximal antimicrobial activity^{23, 49, 50}.

Applicants build upon this work to develop an evolutionary algorithm that searches over the fitness landscape of guide sequences by performing iterative rounds of fitness evaluation, selection, and mutation⁵⁰.

First, Applicants initialize a population of guide sequences G₀ by extracting all of the guide-length sequences at the genomic site of interest. Then, Applicants use the objective function ƒ(g|T) to evaluate the fitness of each of these candidate guides. Applicants sample S parent guides from this population with a probability

$${q}_{i} = \frac{{e}^{β \cdot f ( {g}_{i} \vert T )}}{{\sum }_{i = 1}^{\vert G \vert} {e}^{β \cdot f ( {g}_{i} \vert T )}} ,$$

where β represents the selection intensity⁵⁰. Applicants then mutate the randomly-sampled guides with a mutation frequency γ. Specifically, for each position in the guide, Applicants randomly sample a value from the standard uniform distribution, xᵢ˜Uniform(0,1). If xᵢ<γ, Applicants mutate the nucleotide at that position (e.g., ‘A’) to one of the other nucleotides (e.g., ‘C’, ‘G’, or ‘U’) with uniform probability. Finally, Applicants add the resulting child guides to the guide population. Applicants repeat these rounds of fitness evaluation, selection, and mutation for several generations until the limit on the number of calls to the objective function, L, is exceeded. Applicants return the guide sequences in the final population.

Throughout the search process, the algorithms in BADGERS are capable of efficiently recognizing the positions at which fitness increases if the guide diverges from the consensus sequence, but they don't explicitly evaluate each possible nucleotide at those positions. Thus, after the last generation (of the evolutionary algorithm) and after the last outer round (of the WGAN-AM algorithm), the algorithms perform a local search, where they identify positions at which the generated guide differs from the consensus, mutate the guide to the other possible nucleotides at each of these positions, and then select the resulting guide with the best fitness.

A pseudocode overview of the evolutionary algorithm is available in FIG. 11, and a high-level schematic is presented in FIG. 4d.

### CbAS and AdaLead Search Algorithms

To computationally evaluate the performance of other search algorithms used in the field of biological sequence design in this domain, Applicants applied CbAS³⁴ and AdaLead³⁵ to design guides for both objectives.

Applicants implemented and tested all models using TensorFlow 2.8.0⁵¹ and FLEXS 0.2.8³⁵ in Python 3.7.10.

### Hyperparameter Search

Applicants implemented a random search procedure to determine the optimal hyperparameters for the design algorithms. Because Applicants developed two model-directed exploration algorithms (WGAN-AM and evolutionary) and Applicants have two objective functions (f_{M}(g|T) and f_{D}(g|T₁,T₂)), four sets of hyperparameters were determined by random search.

### Multi-Target Detection Objective Function

For the multi-target detection objective function, f_{M}(g|T), Applicants constructed a random search dataset of genomes from NCBI's GenBank database⁵². Applicants randomly sampled ten viral families from a list of all viral families that have at least one vertebrate-infecting species. Then, within each of these ten families, Applicants randomly sampled one vertebrate-infecting species that has at least 100 complete genomes. The ten selected viral species were Primate T-lymphotropic virus 1 (NCBI taxonomic identifier (taxid): 194440), avian orthoreovirus (taxid: 38170), alphapapillomavirus 10 (taxid: 333754), yellow fever virus (taxid: 11089), eastern equine encephalitis virus (taxid: 11021), pigeon circovirus (taxid: 1414603), SFTS phlebovirus (taxid: 1933190), human respovirus 3 (taxid: 11216), human metapneumovirus (taxid: 162145), and Betacoronavirus 1 (taxid: 694003). For each of these ten viral species, Applicants used ADAPT6 to compile and curate complete genomes and used MAFFT53 create an alignment of the curated genomes. Applicants randomly selected ten guide-length sites from each of the ten alignments, and extracted all of the sequences at each genomic site to create a target set T. In summary, Applicants constructed 100 target sets from 100 genomic sites across ten different viral species-allowing the hyperparameter search process to reflect viral diversity.

Applicants created 100 sets of hyperparameters for the algorithms by randomly sampling from the distributions listed below. For each set of hyperparameters, Applicants ran the WGAN-AM and evolutionary algorithms across all 100 target sets, and computed the mean fitness of the guides

$$( \frac{1}{100} {\sum }_{i = 1}^{100} {f}_{M} ( g \vert {T}_{i} ) )$$

across the target sets for a set of targets Tᵢ at each site). Applicants ultimately selected the set of hyperparameters that the maximized mean guide fitness.

### Variant Identification Objective Function

For the variant identification objective function, f_{D}(g|T₁,T₂), Applicants created a synthetic random search dataset. Applicants randomly generated 50 DNA sequences of length 150 to form the on-target sets

$${T}_{1}^{1} , … , {T}_{1}^{50} .$$

For each of these DNA sequences, Applicants mutated the base in the center of the sequence (position 75) to another randomly-selected base. These mutated DNA sequences comprised the off-target sets,

$${T}_{2}^{1} , … , {T}_{2}^{50} .$$

These pairs or on-target and off-target sets

$$( {T}_{1}^{1} , {T}_{2}^{1} ) , … , ( {T}_{1}^{50} , {T}_{2}^{50} )$$

represent two different sequences, one with a single nucleotide mutation and one without a single nucleotide mutation.

Applicants created 100 sets of hyperparameters the algorithms by randomly sampling from the distributions listed below. Furthermore, since the variant identification objective function, f_{D}(g|T₁,T₂), has hyperparameters as well, Applicants created 100 sets of objective function hyperparameters by randomly sampling from the distributions also listed below.

For each set of hyperparameters, Applicants ran the algorithms across the 50 pairs of on-target and off-target sets

$$( {T}_{1}^{1} , {T}_{2}^{1} ) , … , ( {T}_{1}^{50} , {T}_{2}^{50} )$$

in a sliding window fashion so that Applicants would examine a guide placing the SNP at each position of the guide (window length=28 nt, window stride=1 nt; selecting the optimal of the 28 guides) to design 50 diagnostic guides. Because the objective function f_{D}(g|T₁,T₂) has its own hyperparameters o, a, k, and r, it would not be appropriate to directly compare the fitness of the generated guides across the different hyperparameter sets. Since the goal is to design guides that have high on-target activity and low off-target activity, Applicants selected the set of hyperparameters that maximized the mean difference between predicted on-target and off-target activity

$$\frac{1}{50} {\sum }_{i = 1}^{50} [ A ( g \vert {T}_{1}^{i} ) - A ( g \vert {T}_{2}^{i} ) ]$$

while simultaneously generating guides with a high predicted on-target activity

$$A ( g \vert {T}_{1}^{1} , … , A ( g \vert {T}_{1}^{50} ) > - 1.7 .$$

For both objectives, the evolutionary, AdaLead, and CbAS algorithms were provided a fixed number of model evaluations (L=2000). Since the WGAN-AM algorithm's hyperparameters influence L, a hyperparameter set was chosen such that the algorithm had L within 10% of 2000.

### List of Hyperparameters and Search Intervals

WGAN-AM algorithm parameters determined through random search:
Number of starting points, rₒᵤₜₑᵣ˜uniform in [5, 35] for multi-target detection objective, rₒᵤₜₑᵣ˜uniform in [5, 15] for variant identification objective
Number of steps in each search of the latent space, rᵢₙₙₑᵣ˜uniform in [50, 325] for multi-target detection objective, rᵢₙₙₑᵣ˜uniform in [50, 230] for variant identification objective
optimizer learning rate, α˜log-uniform in [10-2, 102]

Evolutionary algorithm parameters determined through random search:
Selection intensity, β˜log-uniform in [10-1.2, 102.2] for multi-target detection objective, β˜log-uniform in [10-2, 102] for variant identification objective
Mutation rate, γ˜uniform in

$$[ \frac{0}{28} , \frac{10}{28} ]$$

for multi-target detection objective, γ˜uniform in

$$[ \frac{0}{28} , \frac{2}{28} ]$$

for variant identification objective
Fraction of children in new generation, j˜uniform in [0.1, 0.9]
Population size, S˜uniform in [50, 300] for multi-target detection objective, S˜uniform in [50, 250] for variant identification objective

AdaLead parameters determined through random search:
Expected recombination partners per sequence, ρ˜discrete uniform in [0, 2]
Recombination rate, j˜uniform in [0, 0.5]
Expected mutations to sequence, ω˜discrete uniform in [1, 10] for multi-target detection objective, ω˜discrete uniform in [1, 2] for variant identification objective
Fractional threshold κ˜uniform in [0, 0.5]

CbAS parameters determined through random search:
Quantile threshold, Q˜uniform in [0.5, 0.99]

Variant identification objective function parameters determined through random search:
α˜uniform in [0.1, 9.1]
k˜uniform in [−4,−1]
r˜uniform in [0.1, 10.1]
o˜uniform in [−4.5,−1]

### Computational Benchmarking of Model-Directed Exploration Algorithms

To characterize the ability of the WGAN's generator network to introduce mismatches at different positions in the guide and between different alleles, Applicants ran a simulation. Applicants computed the consensus sequence at each of the 100 genomic sites used in the multi-target detection random search. For each consensus sequence C₁, . . . , C₁₀₀, Applicants randomly sampled 500 latent variables z˜N(0, 1) and generated 500 guides conditioned on that consensus sequence using the generator network G(z|Cᵢ). Applicants computed the Euclidean norm of the latent variable as well as the Hamming distance between the WGAN-AM generated guide and the consensus sequence, which is the number of positions at which the two guides have different nucleotides.

Applicants found that that as the Euclidean norm of the latent variable z increases, the guide generated by the WGAN's generator network has a higher Hamming distance from the sequence that it was conditioned upon. In other words, as the latent variable z gets farther from the origin, the generator introduces more mutations into the generated guide. This property is illustrated in FIG. 21b. Thus, the latent space z has an interpretable biological meaning.

Additionally, to benchmark the algorithms' ability to design guides for the variant identification objective, Applicants created a synthetic benchmarking dataset. In order to avoid biasing the sequence composition towards a limited set of pathogen genomes and evaluate the performance of the methods across a large distribution of sequences, Applicants randomly generated 100 pairs of DNA sequences, each pair consisting of targets that differ by one nucleotide. The algorithms in BADGERS were run on this dataset and the results are shown in FIG. 5b-d.

### Designing Diagnostic Guides for Experiments

Applicants applied the model-directed exploration algorithms to design diagnostic guides for both the multi-target detection and variant identification applications.

### Multi-Target Detection Experiments

For the multi-target detection application, Applicants selected five RNA viruses that are of public health interest and cause significant morbidity and mortality globally. Applicants used ADAPT6 to download and curate complete genomes for dengue virus (taxid: 12637), influenza A virus (segment 2, taxid: 11320), and enterovirus B (taxid: 138949) on Aug. 30, 2021 from NCBI's GenBank52. For SARS-CoV-2, Applicants downloaded all complete genomes available on GISAID54 on Jun. 28, 2021, removed the sequences flagged as ‘low quality’, and randomly sampled 10,000 of the genomes. For Lassa virus (segment S, taxid: 11620), Applicants directly downloaded complete genomes from GenBank52 using the following search criteria: ‘txid11620 [Organism: exp] AND “segment S” AND “complete sequence”’ on Aug. 31, 2021. Applicants used MAFFT to generate alignments for these five viral species53. The predictive models (R(g|T) and C(g|T)) employed in this work require an input of a 48 nt target sequence (a 28 nt guide-binding target sequence plus 10 nt of context on each side). Thus, Applicants extracted 48 nt sliding windows along the genome and considered the target set T at that genomic site. Applicants removed the genomic sites with less than 10 valid sequences (sequences that are a contiguous 48 nt and have no ambiguity or gaps). Both of the model-based exploration algorithms were run on all of these target sets using the multi-target objective function, f_{M}(g|T), to design guides for these genomic sites.

To benchmark the performance of the model-based exploration algorithms against current methods, Applicants used ADAPT to design guides at each of these genomic sites. ADAPT is software suite that employs machine learning and combinatorial optimization to design diagnostic guides and is a state-of-the-art in the field6. When running ADAPT, Applicants used the arguments ‘-w 28 -gl 28 --predict-cas13a-activity-model --obj maximize-activity -hgc 1 --cluster-threshold 0.3’ with SEARCH-TYPE set to sliding-window, such that the CNN-based predictive model was used and ADAPT was restricted to designing one guide at each site. Applicants refer to this usage of ADAPT as “model-based choice” (MBC), since the predictive model is being used to select a maximally fit sequence present in the ground set.

Furthermore, Applicants computed the consensus of the sequence alignment at each of these genomic sites to represent a simple baseline approach to guide sequence design. These guides are called “consensus guides” throughout the text.

Applicants sought to appropriately compare the performance of the guides designed by the baseline methods (MBC and the consensus) with the performance of the guides designed by the model-directed exploration algorithms (WGAN-AM and evolutionary). It is not tractable to experimentally test the tens of thousands of diagnostic guides designed. Thus, for every genomic site, Applicants computed a relative performance metric, RPᵢ, the summed fitness of the guides designed by the model-directed exploration algorithms subtracted by the summed fitness of the guides designed by the baseline methods.

$${RP}_{i} = ( f ( {g}_{WGAN} \vert {T}_{i} ) + f ( {g}_{evolutionary} \vert {T}_{i} ) ) - ( f ( {g}_{consensus} \vert {T}_{i} ) + f ( {g}_{MBC} \vert {T}_{i} ) )$$

Previous studies^{6,17} have shown that LwaCas13a exhibits reduced activity when targeting a genomic sites with a G at the PFS, so Applicants only considered genomic sites with a non-G at the PFS for experimental testing. For each viral species, all genomic sites with a non-G at the PFS were sorted into quantiles by their respective RPᵢ, so genomic sites with the highest RPᵢ were in the fourth quartile. Applicants randomly sampled two sites from the fourth quartile, and randomly sampled one site from the first quartile for each viral species. This method for selecting genomic sites allows for experimental testing of sites at which the guides designed by the model-directed exploration algorithms were likely to have superior performance than the guides designed by previous methods, as well as sites at which guides designed by previous methods were likely to have similar or better performance than the guides designed by the model-directed exploration algorithms. Each panel with experimental multi-target detection results shows one such site.

To computationally characterize guide performance, Applicants computed the coverage of the guides designed by the algorithms in BADGERS and the baseline methods across different genomic windows in the viral pathogens, as shown in FIG. 5a and FIG. 9.

A guide g was considered to detect a target if it was predicted to be active by the classification model (C(g|t)>0.577467) and was predicted to be in the top quartile of activity by the regression model (R(g|t)>1.2801363), based on the “highly active” criterion used in ref. 6. The coverage was computed by determining the percentage of targets a guide detects in the given target set. To visualize these results in FIG. 5a and FIG. 9, the guide designed by each method in each genomic window (FIG. 5a, top) or annotated region (FIG. 5a, bottom) with the maximum predicted coverage was selected. The percentage of targets detected by this guide is shown in the graphs.

Applicants computed the predicted guide activity across all targetable genomic sites in these pathogens, as shown in FIG. 5b and FIG. 10a. Targetable genomic sites are defined as sites at which the baseline methods had meaningful activity

$$( \frac{A ( {g}_{consensus} \vert T ) + A ( {g}_{MBC} \vert T )}{2} > - 2.5 ;$$

in practice, it would not make sense to target sites that fall below this minimal threshold). In these figures and in all boxplots throughout the manuscript, the box represents the first and third quartiles (Q1 and Q3) and the whiskers extend to from Q1−1.5·IQR to Q3+1.5·IQR.

### Variant Identification Experiments

For the variant identification application, Applicants sought to design diagnostic guides that could differentiate between dengue virus (DENV) serotypes 1-4 and the SARS-CoV-2 WHO-designated variants of concern. For DENV, Applicants downloaded all available complete genomes for serotypes 1-4 from the Virus Pathogen Database and Analysis Resource (ViPR)⁵⁵ on Feb. 1, 2022. Applicants used MAFFT53 to generate an alignment of the genomes.

For SARS-CoV-2, Applicants downloaded the ‘full length variant alignment’ from GISAID on Jan. 22, 2022. This alignment contains representative sequences for the targeted SARS-CoV-2 lineages: Wuhan reference, Alpha (B.1.1.7), Beta (B.1.351), Gamma (P.1), Delta (B.1.617.2), Omicron (BA.1), and Omicron (BA.2). The GISAID accessions for these reference genomes are EPI ISL 402124, EPI ISL 674612, EPI ISL 940877, EPI ISL 2777382, EPI ISL 1758376, EPI ISL 6914029, and EPI ISL 7190366, respectively. Applicants referenced analyses on the CoVariants56 and outbreak.info⁵⁷ websites to confirm that the insertions, deletions, and mutations targeted by the guides were highly conserved and were present in at least 85% of genomic sequences of each variant.

The model-directed exploration algorithms were run on the DENV and SARS-CoV-2 alignments described above to design guides that were specific to each serotype/genotype/lineage.

To benchmark the performance of the model-directed exploration algorithms for variant identification of lineages (beyond a single SNP), Applicants implemented a baseline approach. This baseline approach designs guide sequences by finding the 28-nt subsequence of the on-target consensus that has the highest Hamming distance from the off-target consensus and has an average nucleotide identity of at least 90% across the on-target genomes. This approach mimics the strategy (usually performed by hand) of identifying regions that are conserved among the on-target genomes but divergent from the off-targets.

For the antimalarial resistance panel, Applicants downloaded reference sequences for the P. falciparum genes Pfmdr1, Pfcrt, Pfk13, Pfdhps from GenBank accessions HQ215532.1, LC498250.1, KT328114.1, and KE123491.1, respectively. For the K417N/T identification task, Applicants used the same SARS-CoV-2 sequences that Applicants previously downloaded from GISAID, as described above. For the Zika virus S139N identification task, Applicants downloaded the reference sequence from GenBank accession MT483911.1. Applicants extracted the 250 nt genomic regions encapsulating the sites of interest in each gene and used them as the target sequences.

The model-directed exploration algorithms were run to design guides for each of the 4 single nucleotide polymorphisms (SNPs) of interest in the P. falciparum genome, the K417N/T SNP in SARS-CoV-2, and the S139N SNP in Zika virus. Applicants also sought to benchmark the algorithms against a baseline. The “synthetic mismatch” strategy (where the SNP is placed at position 26 of the protospacer and a mismatch at position 24 is introduced against both the derived and ancestral targets) is currently the standard approach for designing Cas13a guides that identify SNPs^{17, 58}. The synthetic mismatch creates a double mismatch against the off-target sequence (positions 24 and 26), but only one mismatch against the on-target (position 24). Applicants employed the synthetic mismatch strategy to design guides that target these 4 SNPs of interest, to enable benchmarking of the performance of the methods against the current standard.

### Tag-Adjacent Mismatch Experiments

As discussed in the main text, the model-directed exploration algorithms often introduce a mismatch at position 28 in the guide when targeting a genomic site with G nucleotide at the protospacer-flanking site (FIG. 5). Applicants refer to this mismatch the “tag-adjacent mismatch” (TAM) and hypothesized that it may enhance guide-target activity.

To determine if this was the case, Applicants designed an experimental library consisting of targets that were representative of viral sequence diversity. Applicants randomly sampled four viral families from a list of all viral families that have at least one vertebrate-infecting species. Then, within each of these four families, Applicants randomly sampled one vertebrate-infecting species that has at least 100 complete genomes. The four selected viral species were primate tick-borne encephalitis virus (NCBI taxid: 11084), human mastadenovirus D (taxid: 130310), hepatitis B virus (taxid: 10407), and Zaire ebolavirus (taxid: 186538). For each of these viral species, Applicants downloaded all available complete genomes from GenBank52 on Feb. 15, 2022 and used MAFFT53 to create an alignment. Applicants randomly selected a genomic site with a G nucleotide at the PFS in each of the four alignments and used this as the target sequence. Applicants mutated the G nucleotide at the PFS in the original target sequence to create three additional targets with a non-G nucleotide at the PFS. Furthermore, since the nucleotide directly 3′ of a site with a G at the PFS also impacts activity⁶, Applicants additionally mutated the original target sequence to create targets that have an anti-tag region of GC, GG, GA, and GT.

For each of the four species, Applicants designed the guide sequence without a TAM by simply extracting the 28 nt protospacer-binding region from the original target sequence. Applicants also designed three guide sequences with the TAM by mutating the nucleotide at position 28 of the guide's protospacer to the other three possible bases at that position.

### Preparing Target Sequences for Experiments

To experimentally test the performance of the guides, Applicants first designed target sequences against which Applicants would test guides. Applicants designed these targets to be representative of viral genomic diversity. Applicants ran ADAPT's pick test targets.py program developed in ref. 6, with the following arguments: --num-representative-targets 5 --min-target-len 250. This script extracts a region of the alignment that encapsulates the guide-binding sites and is at least 250 nt long. Then, it clusters the resulting sequences and determines the medoid of each cluster, which are used as the representative target sequences. At all of the genomic sites Applicants experimentally tested, the targets represented at least 95% of the total genomic diversity.

Applicants added a T7 promoter sequence (5′-GAAATTAATACGACTCACTATAGGG-3′ (SEQ ID NO: 1)) followed by a positive control sequence (5′-CACTATAGGGGCTCTAGCGACTTCTTTAAATAGTGGCTTAAAATAAC-3′ (SEQ ID NO: 2)) to 5′ end of every target sequence. In each experiment, Applicants included a crRNA with a protospacer that was complementary to this positive control sequence (5′-GCTCTAGCGACTTCTTTAAATAGTGGCT-3′ (SEQ ID NO: 3)). This positive control enabled verification that the target sequences were synthesized correctly. P. falciparum's genome may pose DNA synthesis challenges because it is GC-poor, so Applicants added a second positive control sequence (5′-GAATGGAAGCACCGAGAGTATATGAAGATCTTCATGTGTGCAAAAGAATGGTAAAG CAGAGAAGGAGC-3′ (SEQ ID NO: 4)) to the 3′ end of all the P. falciparum target sequences. In each experiment that had P. falciparum targets, Applicants included crRNA with a protospacer that was complementary to this positive control (5′-TTCTTTTGCACACATGAAGATCTTCATAT-3′ (SEQ ID NO: 5)).

### Preparing Guide Sequences for Experiments

At each genomic site, the WGAN-AM algorithm outputs rₒᵤₜₑᵣ guides and the evolutionary algorithm outputs S guides; the WGAN-AM guide sequence and the evolutionary guide sequence with the highest predicted fitness were used for experimental testing. All of the methods employed in this work (including the model-based exploration algorithms and the baseline methods) output guide sequences in the frame of Cas13's protospacer. Thus, to prepare the guide sequences for experiments, Applicants took the reverse complement of them to transform them to the frame of the CRISPR-Cas13 spacer and added the LwaCas13 direct repeat (5′-GAUUUAGACUACCCCAAAAACGAAGGGGACUAAAAC-3′ (SEQ ID NO: 6)) to the 5′ end of the crRNA sequence.

### Experimentally Evaluating Diagnostic Guide Designs

### Experimental Methods

Applicants used the mCARMEN⁴⁰ platform to experimentally test the performance of diagnostic guides. Briefly, mCARMEN is a CRISPR-based diagnostic technology that uses a Fluidigm microfluidic chip to enable highly multiplexed testing of dozens of diagnostic guides against dozens of RNA targets. Applicants followed the protocol described in the “General mCARMEN procedures” section of ref. 40, with the following modifications:

All DNA targets were ordered as gBlocks from Integrated DNA Technologies. All crRNAs and the quenched synthetic fluorescent RNA reporter (FAM/rUrUrUrUrUrUrU/3IABKFQ/) were also ordered from Integrated DNA Technologies.

The DNA targets were serially diluted to the concentrations of 1010, 109, and 108 copies/μL, and 1.43 μL of each target served as input to each sample mix. The quenched synthetic fluorescent reporter was included in the sample mix at a concentration of 500 nM. The crRNAs were included in the assay mixes at a concentration of 212.5 nM, and LwaCas13 from GenScript was included in the assay mix at a concentration of 42.5 nM.

After chip loading, the Fluidigm Biomark HD was set to a constant temperature of 37° C. and was used to image the IFC on the FAM and ROX channels every five minutes for three hours.

### Analysis of Experimental Data

In order to robustly characterize the performance of each of the guide-target pairs, Applicants computed reference-normalized background-subtracted fluorescence values, as was done in refs. 40 and 6.

$$reference - normalized fluorescence = \frac{{S}_{t} - {S}_{0}}{{R}_{t} - {R}_{0}}$$

where Sₜ is guide-target pair's FAM signal at time point t, S₀ is the guide-target pair's FAM signal at time point 0, Rₜ is guide-target pair's reference ROX signal at time point t, and R₀ is the guide-target pair's reference ROX signal at time point 0.

To compute the reference-normalized background-subtracted fluorescence of each guide-target pair at time t, Applicants subtracted the reference-normalized fluorescence of the no-template control at time t from the reference-normalized fluorescence of the guide-target pair at time t. Thus, if a guide-target pair has a reference-normalized background-subtracted fluorescence greater than 0, it has a fluorescence that is higher than the no-template control signal.

In the heatmaps of fluorescence, Applicants plotted the reference-normalized background-subtracted fluorescence at the one hour timepoint. In the kinetic curves of fluorescence, Applicants plotted all the reference-normalized background-subtracted fluorescence values collected throughout the course of the reaction (t=0 to 180 minutes).

### REFERENCES FOR EXAMPLE 1

[1] Jabado, O. J. et al. Greene SCPrimer: a rapid comprehensive tool for designing degenerate primers from multiple sequence alignments. Nucleic Acids Research 34, 6605-6611 (2006).
[2] Kreer, C. et al. openPrimeR for multiplex amplification of highly diverse templates. J. Immunol. Methods 480, 112752 (2020).
[3] Duitama, J. et al. PrimerHunter: a primer design tool for PCR-based virus subtype identification. Nucleic Acids Res. 37, 2483-2492 (2009).
[4] Brodin, J. et al. A multiple-alignment based primer design algorithm for genetically highly variable DNA targets. BMC Bioinformatics 14, 255 (2013).
[5] Varliero, G., Wray, J., Malandain, C. & Barker, G. PhyloPrimer: a taxon-specific oligonucleotide design platform. Peer J 9, e11120 (2021).
[6] Metsky, H. C. et al. Designing sensitive viral diagnostics with machine learning. Nat Biotechnology 40, 1123-1131 (2022).
[7] Gupta, A. & Zou, J. Feedback GAN for DNA optimizes protein functions. Nature Machine Intelligence 1, 105-111 (2019).
[8] Repecka, D. et al. Expanding functional protein sequence spaces using generative adversarial networks. Nature Machine Intelligence 3, 324-333 (2021).
[9] Shin, J.-E. et al. Protein design and variant prediction using autoregressive generative models. Nat. Commun. 12, 2403 (2021).
[10] Watson, J. L. et al. Broadly applicable and accurate protein design by integrating structure prediction networks and diffusion generative models. bioRxiv 2022.12.09.519842 (2022).
[11] Strokach, A. & Kim, P. M. Deep generative modeling for protein design. Curr. Opin. Struct. Biol. 72, 226-236 (2022).
[12] Madani, A. et al. Large language models generate functional protein sequences across diverse families. Nature Biotechnology (2023).
[13] Taskiran, I. I. et al. Cell type directed design of synthetic enhancers. Nature (2023).
[14] de Almeida, B. P. et al. Targeted design of synthetic enhancers for selected tissues in the drosophila embryo. Nature (2023).
[15] Gosai, S. J. et al. Machine-guided design of synthetic cell type-specific cis-regulatory elements. bioRxiv (2023).
[16] Zhao, D. et al. Imperfect guide-RNA (igRNA) enables CRISPR single-base editing with ABE and CBE. Nucleic Acids Res 50, 4161-4170 (2022).
[17] Gootenberg, J. S. et al. Nucleic acid detection with CRISPR-Cas13a/C2c2. Science 356, 438-442 (2017).
[18] Arizti-Sanz, J. et al. Simplified cas13-based assays for the fast identification of SARS-CoV-2 and its variants. Nat. Biomed. Eng. 6, 932-943 (2022).
[19] Kaminski, M. M., Abudayyeh, O. O., Gootenberg, J. S., Zhang, F. & Collins, J. J. CRISPR-based diagnostics. Nat. Biomed. Eng. 5, 643-656 (2021).
[20] Killoran, N., Lee, L. J., Delong, A., Duvenaud, D. & Frey, B. J. Generating and designing dna with deep generative models. arXiv (2017). 1712.06148.
[21] Zrimec, J. et al. Controlling gene expression with deep generative design of regulatory DNA. Nat. Commun. 13, 5099 (2022).
[22] Sample, P. J. et al. Human 5′ UTR design and variant effect prediction from a massively parallel translation assay. Nat. Biotechnol. 37, 803-809 (2019).
[23] Porto, W. F. et al. In silico optimization of a guava antimicrobial peptide enables combinatorial exploration for peptide design. Nature Communications 9 (2018).
[24] Fox, R. Directed molecular evolution by machine learning and the influence of nonlinear interactions. J. Theor. Biol. 234, 187-199 (2005).
[25] Granados, A., Peci, A., McGeer, A. & Gubbay, J. B. Influenza and rhinovirus viral load and disease severity in upper respiratory tract infections. Journal of Clinical Virology 86, 14-19 (2017). URL www.sciencedirect.com/science/article/pii/S1386653216306035.
[26] Dayarathna, S. et al. Are viral loads in the febrile phase a predictive factor of dengue disease severity? medRxiv (2023). URL www.medrxiv.org/content/early/2023/07/31/2023.07.31.23293412. www.medrxiv.org/content/early/2023/07/31/2023.07. 31.23293412.full.pdf.
[27] Yuan, L. et al. A single mutation in the prm protein of zika virus contributes to fetal microcephaly. Science 358, 933-936 (2017).
[28] Apinjoh, T. O., Ouattara, A., Titanji, V. P. K., Djimde, A. & Amambua-Ngwa, A. Genetic diversity and drug resistance surveillance of Plasmodium falciparum for malaria elimination: is there an ideal tool for resource-limited sub-saharan africa? Malar. J. 18, 217 (2019).
[29] Carter, T. E. et al. Evaluation of dihydrofolate reductase and dihydropteroate synthetase genotypes that confer resistance to sulphadoxine-pyrimethamine in Plasmodium falciparum in haiti (2012).
[30] Quan, H. et al. High multiple mutations of Plasmodium falciparum-resistant genotypes to sulphadoxine-pyrimethamine in lagos, nigeria. Infect Dis Poverty 9, 91 (2020).
[31] Yoshida, N., Yamauchi, M., Morikawa, R., Hombhanje, F. & Mita, T. Increase in the proportion of Plasmodium falciparum with kelch13 C580Y mutation and decline in pfcrt and pfmdr1 mutant alleles in papua new guinea. Malar. J. 20, 410 (2021).
[32] Hyde, J. E. Drug-resistant malaria. Trends Parasitol. 21, 494-498 (2005).
[33] Harvey, W. T. et al. SARS-CoV-2 variants, spike mutations and immune escape. Nat Rev Microbiol 19, 409-424 (2021).
[34] Brookes, D., Park, H. & Listgarten, J. Conditioning by adaptive sampling for robust design. In Chaudhuri, K. & Salakhutdinov, R. (eds.) Proceedings of the 36th International Conference on Machine Learning, vol. 97 of Proceedings of Machine Learning Research, 773-782 (PMLR, 2019).
[35] Sinai, S. et al. AdaLead: A simple and robust adaptive greedy search algorithm for sequence design. arXiv (2020). 2010.02141.
[36] Tambe, A., East-Seletsky, A., Knott, G. J., Doudna, J. A. & O'Connell, M. R. RNA binding and HEPN-Nuclease activation are decoupled in CRISPR-Cas13a. Cell Rep. 24, 1025-1036 (2018).
[37] Abudayyeh, O. O. et al. RNA targeting with CRISPR-Cas13. Nature 550, 280-284 (2017).
[38] Abudayyeh, O. O. et al. C2c2 is a single-component programmable RNA-guided RNA-targeting CRISPR effector. Science 353, aaf5573 (2016).
[39] Meeske, A. J. & Marraffini, L. A. RNA guide complementarity prevents Self-Targeting in type VI CRISPR systems. Mol. Cell 71, 791-801.e3 (2018).
[40] Welch, N. L. et al. Multiplexed CRISPR-based microfluidic platform for clinical testing of respiratory viruses and identification of SARS-CoV-2 variants. Nature Medicine (2022).
[41] Bock, C. et al. High-content CRISPR screening. Nat Rev Methods Primers 2 (2022).
[42] Huang, X., Yang, D., Zhang, J., Xu, J. & Chen, Y. E. Recent Advances in Improving Gene-Editing Specificity through CRISPR-Cas9 Nuclease Engineering. Cells 11 (2022).
[43] Saha, K. Accounting for diversity in the design of CRISPR-based therapeutic genome editing. Nat Genet 55, 6-7 (2023).
[44] Shanehsazzadeh, A. et al. Unlocking de novo antibody design with generative artificial intelligence. bioRxiv (2023). URL www.biorxiv.org/content/early/2023/01/09/2023.01.08.523187. www.biorxiv.org/content/early/2023/01/09/2023.01. 08.523187.full.pdf.
[45] Goodfellow, I. J. et al. Generative adversarial networks. arXiv (2014). 1406.2661.
[46] Mirza, M. & Osindero, S. Conditional generative adversarial nets. arXiv (2014). URL arxiv.org/abs/1411.1784.
[47] Gulrajani, I., Ahmed, F., Arjovsky, M., Dumoulin, V. & Courville, A. Improved training of wasserstein gans. arXiv preprint arXiv: 1704.00028 (2017).
[48] Kingma, D. P. & Ba, J. Adam: A method for stochastic optimization. arXiv (2014). 1412.6980.
[49] Yoshida, M. et al. Using evolutionary algorithms and machine learning to explore sequence space for the discovery of antimicrobial peptides. Chem 4, 533-543 (2018).
[50] Sinai, S. & Kelsic, E. D. A primer on model-guided exploration of fitness landscapes for biological sequence design. arXiv (2020). 2010.10614.
[51] Mart'ιn Abadi et al. TensorFlow: Large-Scale machine learning on heterogeneous systems (2015).
[52] Federhen, S. The NCBI taxonomy database. Nucleic Acids Research 40, D136-43 (2012).
[53] Katoh, K. & Standley, D. M. MAFFT multiple sequence alignment software version 7: improvements in performance and usability. Molecular Biology and Evolution 30, 772-780 (2013).
[54] Shu, Y. & McCauley, J. GISAID: Global initiative on sharing all influenza data from vision to reality. Euro surveillance: bulletin Europeen sur les maladies transmissibles=European communicable disease bulletin 22 (2017).
[55] Pickett, B. E. et al. ViPR: An open bioinformatics database and analysis resource for virology research. Nucleic Acids Research 40 (2011).
[56] Hodcroft, E. B. CoVariants: SARS-CoV-2 mutations and variants of interest. covariants.org/.
[57] Mullen, J. L. et al. outbreak.info. outbreak.info/.
[58] Kellner, M. J., Koob, J. G., Gootenberg, J. S., Abudayyeh, O. O. & Zhang, F. SHERLOCK: nucleic acid detection with CRISPR nucleases. Nat. Protoc. 14, 2986-3012 (2019).

### Supplementary Note 1

This note estimates the size of the sequence landscape that must be searched over to generate artificial guides.

The total number of possible 28-nt guides is 4²⁸≈7×10¹⁶.

Optimally fit guide sequences would be nearby in sequence space to the complement of their target sequence. If M is the upper limit on the number of mismatches an optimal guide can have to this sequence, there are

$${\sum }_{i = 0}^{M} ( \begin{matrix}28 \\ i\end{matrix} ) \cdot {3}^{i}$$

in this target-adjacent guide set. This totals to ˜2.5×10⁷ and ˜2.9×10⁹ for M=5 and M=7, respectively. In many applications, Applicants would explore this space not just once, but at every site in a targeted genome (tens to hundreds of thousands of sites for a typical viral genome); moreover, if a set of targets has high variation, Applicants would explore the space around many observed alleles of the target. It would be inefficient to explore this vast space of potential guide sequences with an exhaustive, brute-force search.

### REFERENCES FOR EXAMPLE 1—SUPPLEMENTARY NOTE 1

[1] Metsky, H. C. et al. Designing sensitive viral diagnostics with machine learning. Nat Biotechnology 40, 1123-1131 (2022).
[2] Killoran, N., Lee, L. J., Delong, A., Duvenaud, D. & Frey, B. J. Generating and designing dna with deep generative models. arXiv (2017). 1712.06148.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A computer-implemented method to generate one or more target binding oligonucleotides, comprising:
a) processing one or more target nucleic acid sequences with a deployed oligonucleotide generating network and
b) generating, by the deployed oligonucleotide generating network, one or more engineered target binding oligonucleotides, wherein the one or more engineered target binding oligonucleotides comprise one or more mismatches.

2. The method of claim 1, further comprising:
c) preparing the one or more engineered target binding oligonucleotides.

3. The method of claim 1, wherein the target binding oligonucleotide is a guide nucleic acid sequence, small interfering RNA (siRNA), microRNA (miRNA), diagnostic primer nucleic acid sequence, probe nucleic acid sequence, peptide nucleic acid (PNA), or locked nucleic acid (LNA).

4. The method of claim 3, wherein the target binding oligonucleotide is a guide nucleic acid sequence.

5. The method of claim 1, wherein the one or more mismatch is a nucleotide not complementary to a corresponding nucleotide of at least one, at least 25%, at least 50%, or at least all of the target nucleic acid sequences;
optionally wherein two or more mismatches are within a 60-, within a 50-, within a 40-within a 30-, within a 20-, within a 10-, or within a 5-nucleotide range; and
optionally wherein the target binding oligonucleotide comprises a tag adjacent mismatch.

6-7. (canceled)

8. The method of claim 1, wherein the target oligonucleotide comprises one or more polymorphism, optionally wherein the mismatch is within 1, 2, 3, 4, 5, 6, 7, 8, 9 or 10 nucleotides from the one or more polymorphism.

9. (canceled)

10. The method of claim 1, wherein the oligonucleotide generating network comprises a neural network, Bayesian network, random forest, diffusion model, autoregression model, matrix factorization, hidden Markov model, support vector machine, K-means clustering, K-nearest neighbor, linear classifiers, logistic classifiers, linear regression models, logistic regression models, or any combination thereof.

11. The method of claim 10, wherein the neural network comprises a deep learning network, a convolutional neural network, or a recurrent neural network;
optionally wherein the deep learning network comprises a generative adversarial network;
optionally wherein the generative adversarial network comprises a Wasserstein Generative Adversarial Network (WGAN);
optionally wherein the WGAN is conditional on the one or more target nucleic acid sequences; and
optionally wherein the oligonucleotide generating network comprises activation maximization.

12-15. (canceled)

16. The method of claim 1, wherein the oligonucleotide generating network comprises an evolutionary network;
optionally wherein the evolutionary network introduces one or more mutations to at least one, at least 25%, at least 50%, at least all target binding oligonucleotides thereby generating a new target binding oligonucleotide;
optionally wherein the one or more mutations occur according to a mutation frequency;
optionally wherein the one or more mutations are random;
optionally wherein the new target binding oligonucleotide is added to the one or more target binding oligonucleotides generating a new set of target binding oligonucleotides and the evolutionary network mutates the new set of target binding oligonucleotides in an iterative process, optionally until a preset number of iterations and/or a preset threshold; and
optionally wherein the evolutionary network comprises a fitness evaluation.

17-21. (canceled)

22. The method of claim 11, wherein the oligonucleotide generating network comprises an objective network;
optionally wherein the objective network generates a target interaction score between the one or more target binding oligonucleotides and the one or more target nucleic acid sequences;
optionally wherein the objective network generates a non-target interaction score between the one or more target binding oligonucleotides, the one or more target nucleic acid sequences, and one or more non-target sequences;
optionally wherein the objective network comprises a logistic regression model;
optionally wherein the objective network comprises an optimizer;
optionally further comprising first training the oligonucleotide generating network with a target interaction score, non-interaction score, or both;
optionally wherein the processing step further comprises processing a target interaction score, non-interaction score, or both and the generating step further comprises generating one or more engineered target binding oligonucleotides with a corresponding target interaction score, non-interaction score, or both; and
optionally further comprising training the objective network with the one or more engineered target binding oligonucleotides with the corresponding target interaction score, non-interaction score, or both.

23-29. (canceled)

30. The method of claim 1, further comprising:
i. transmitting the one or more target binding oligonucleotides and the one or more target nucleic acid sequences to a deployed biological activity network, by one or more computing devices;
ii. processing the one or more target binding oligonucleotides and the one or more target nucleic acid sequences with the deployed biological activity network; and
iii. generating, by the biological activity network, an activity score for the one or more target binding oligonucleotides and the one or more target nucleic acid sequences,
wherein steps i-iii are performed after step b) or c).

31. The method of claim 30, wherein the biological activity network comprises a classification network and regression network;
optionally wherein the classification network generates an active or inactive score;
optionally wherein the regression network generates a level of activity of target binding oligonucleotides;
optionally wherein the activity score is a combination of the classification network and regression network;
optionally wherein the biological activity network comprises a neural network;
optionally wherein the neural network comprises a deep learning network, a convolutional neural network, or a recurrent neural network; and
optionally wherein the neural network is a convolutional neural network.

32-37. (canceled)

38. The method of claim 30, wherein the oligonucleotide generating network and the biological activity network are deployed from individual training machine learning networks, optionally wherein the oligonucleotide generating network and the biological activity network are trained using a learning method individually selected from the group consisting of unsupervised learning, supervised learning, semi-supervised learning, reinforcement learning, transfer learning, incremental learning, curriculum learning, learning to learn, contrastive learning, and any combination thereof.

39. (canceled)

40. A system to generate one or more engineered target binding oligonucleotides, comprising:
a storage device; and
a processor communicatively coupled to the storage device, wherein the processor executes application code instructions that are stored in the storage device to cause the system to:
a) process one or more target nucleic acid sequences with a deployed oligonucleotide generating network and
b) generate one or more engineered target binding oligonucleotides with the deployed oligonucleotide generating network, wherein the one or more engineered target binding oligonucleotides comprise one or more mismatches.

41. The system of claim 40, further comprising:
c) preparing the one or more engineered target binding oligonucleotides.

42. The system of claim 40, wherein the target binding oligonucleotide is a guide nucleic acid sequence, small interfering RNA (siRNA), microRNA (miRNA), diagnostic primer nucleic acid sequence, probe nucleic acid sequence, PNA, or LNA.

43. The system of claim 42, wherein the target binding oligonucleotide is a guide nucleic acid sequence.

44. The system of claim 40, wherein the mismatch is a nucleotide not complementary to a nucleotide of at least one, at least 25%, at least 50%, at least all of the target nucleic acid sequences;
optionally wherein two or more mismatches are within a 60-, within a 50-, within a 40-, within a 30-, within a 20-, within a 10-, or within a 5-nucleotide range; and
optionally wherein the target binding oligonucleotide comprises a tag adjacent mismatch.

45-46. (canceled)

47. The system of claim 40, wherein the target oligonucleotide comprises one or more polymorphism, optionally wherein the mismatch is within 1, 2, 3, 4, 5, 6, 7, 8, 9 or 10 nucleotides from the one or more polymorphism.

48. (canceled)

49. The system of claim 40, wherein the oligonucleotide generating network comprises a neural network, Bayesian network, random forest, diffusion model, autoregression model, matrix factorization, hidden Markov model, support vector machine, K-means clustering, K-nearest neighbor, linear classifiers, logistic classifiers, linear regression models, logistic regression models, or any combination thereof.

50. The system of claim 49, wherein the neural network comprises a deep learning network, a convolutional neural network, or a recurrent neural network;
optionally wherein the deep learning network comprises a generative adversarial network;
optionally wherein the generative adversarial network comprises a Wasserstein Generative Adversarial Network (WGAN);
optionally wherein the WGAN is conditional on the one or more target nucleic acid sequences; and
optionally wherein the oligonucleotide generating network comprises activation maximization.

51-54. (canceled)

55. The system of claim 40, wherein the oligonucleotide generating network comprises an evolutionary network;
optionally wherein the evolutionary network introduces one or more mutations to at least one, at least 25%, at least 50%, at least all target binding oligonucleotides thereby generating a new target binding oligonucleotide;
optionally wherein the one or more mutations occur according to a mutation frequency;
optionally wherein the one or more mutations are random;
optionally wherein the new target binding oligonucleotide is added to the one or more target binding oligonucleotides generating a new set of target binding oligonucleotides and the evolutionary network mutates the new set of target binding oligonucleotides in an iterative process, optionally until a preset number of iterations and/or a preset threshold; and
optionally wherein the evolutionary network comprises a fitness evaluation.

56-60. (canceled)

61. The system of claim 50, wherein the oligonucleotide generating network comprises an objective network;
optionally wherein the objective network generates a target interaction score between the one or more target binding oligonucleotides and the one or more target nucleic acid sequences;
optionally wherein the objective network generates a non-target interaction score between the one or more target binding oligonucleotides, the one or more target nucleic acid sequences, and one or more non-target sequences;
optionally wherein the objective network comprises a logistic regression model;
optionally wherein the objective network comprises an optimizer;
optionally further comprising first training the oligonucleotide generating network with a target interaction score, non-interaction score, or both;
optionally wherein the processing step further comprises processing a target interaction score, non-interaction score, or both and the generating step further comprises generating one or more engineered target binding oligonucleotides with a corresponding target interaction score, non-interaction score, or both; and
optionally further comprising training the objective network with the one or more engineered target binding oligonucleotides with the corresponding target interaction score, non-interaction score, or both.

62-68. (canceled)

69. The system of claim 40, further comprising:
i. transmit the one or more target binding oligonucleotides and the one or more target nucleic acid sequences to a deployed biological activity network;
ii. process the one or more target binding oligonucleotides and the one or more target nucleic acid sequences with the deployed biological activity network; and
iii. generate, by the biological activity network, an activity score for the one or more target binding oligonucleotides and the one or more target nucleic acid sequences, wherein steps i-iii are performed after step b) or c).

70. The system of claim 69, wherein the biological activity network comprises a classification network and regression network;
optionally wherein the classification network generates an active or inactive score;
optionally wherein the regression network generates a level of activity of target binding oligonucleotides;
optionally wherein the activity score is a combination of the classification network and regression network;
optionally wherein the biological activity network comprises a neural network;
optionally wherein the neural network comprises a deep learning network, a convolutional neural network, or a recurrent neural network; and
optionally wherein the neural network is a convolutional neural network.

71-76. (canceled)

77. The system of claim 69, wherein the oligonucleotide generating network and the biological activity network are deployed from individual training machine learning networks, optionally wherein the oligonucleotide generating network and the biological activity network are trained using a learning method individually selected from the group consisting of unsupervised learning, supervised learning, semi-supervised learning, reinforcement learning, transfer learning, incremental learning, curriculum learning, learning to learn, contrastive learning, and any combination thereof.

78. (canceled)

79. A computer program product, comprising:
a non-transitory computer-readable storage device having computer-executable program instructions embodied thereon that when executed by a computer cause the computer to generate one or more engineered target binding oligonucleotides, the computer-executable program instructions comprising:
a) computer-executable program instructions to process one or more target nucleic acid sequences with a deployed oligonucleotide generating network and
b) computer-executable program instructions to generate one or more engineered target binding oligonucleotides with the deployed oligonucleotide generating network, wherein the one or more engineered target binding oligonucleotides comprise one or more mismatches.

80. The computer program product of claim 79, further comprising:
c) preparing the one or more engineered target binding oligonucleotides.

81. The computer program product of claim 79, wherein the target binding oligonucleotide is a guide nucleic acid sequence, small interfering RNA (siRNA), microRNA (miRNA), diagnostic primer nucleic acid sequence, probe nucleic acid sequence, PNA, or LNA.

82. The computer program product of claim 81, wherein the target binding oligonucleotide is a guide nucleic acid sequence.

83. The computer program product of claim 79, wherein the mismatch is a nucleotide not complementary to a nucleotide of at least one, at least 25%, at least 50%, at least all of the target nucleic acid sequences;
optionally wherein two or more mismatches are within a 60-, within a 50-, within a 40-within a 30-, within a 20-, within a 10-, or within a 5-nucleotide range; and
optionally wherein the target binding oligonucleotide comprises a tag adjacent mismatch.

84-85. (canceled)

86. The computer program product of claim 79, wherein the target oligonucleotide comprises one or more polymorphism, optionally wherein the mismatch is within 1, 2, 3, 4, 5, 6, 7, 8, 9 or 10 nucleotides from the one or more polymorphism.

87. (canceled)

88. The computer program product of claim 79, wherein the oligonucleotide generating network comprises a neural network, Bayesian network, diffusion model, autoregression model, random forest, matrix factorization, hidden Markov model, support vector machine, K-means clustering, K-nearest neighbor, linear classifiers, logistic classifiers, linear regression models, logistic regression models, or any combination thereof.

89. The computer program product of claim 88, wherein the neural network comprises a deep learning network, a convolutional neural network, or a recurrent neural network;
optionally wherein the deep learning network comprises a generative adversarial network;
optionally wherein the generative adversarial network comprises a Wasserstein Generative Adversarial Network (WGAN);
optionally wherein the WGAN is conditional on the one or more target nucleic acid sequences; and
optionally wherein the oligonucleotide generating network comprises activation maximization.

90-93. (canceled)

94. The computer program product of claim 79, wherein the oligonucleotide generating network comprises an evolutionary network;
optionally wherein the evolutionary network introduces one or more mutations to at least one, at least 25%, at least 50%, at least all target binding oligonucleotides thereby generating a new target binding oligonucleotide;
optionally wherein the one or more mutations occur according to a mutation frequency;
optionally wherein the one or more mutations are random;
optionally wherein the new target binding oligonucleotide is added to the one or more target binding oligonucleotides generating a new set of target binding oligonucleotides and the evolutionary network mutates the new set of target binding oligonucleotides in an iterative process, optionally until a preset number of iterations and/or a preset threshold; and
optionally wherein the evolutionary network comprises a fitness evaluation.

95-106. (canceled)

107. The computer program product of claim 79, further comprising:
i. transmit the one or more target binding oligonucleotides and the one or more target nucleic acid sequences to a deployed biological activity network;
ii. process the one or more target binding oligonucleotides and the one or more target nucleic acid sequences with the deployed biological activity network; and
iii. generate, by the biological activity network, an activity score for the one or more target binding oligonucleotides and the one or more target nucleic acid sequences,
wherein steps i-iii are performed after step b) or c).

108. The computer program product of claim 107, wherein the biological activity network comprises a classification network and regression network;
optionally wherein the classification network generates an active or inactive score;
optionally wherein the regression network generates a level of activity of target binding oligonucleotides;
optionally wherein the activity score is a combination of the classification network and regression network;
optionally wherein the biological activity network comprises a neural network;
optionally wherein the neural network comprises a deep learning network, a convolutional neural network, or a recurrent neural network; and
optionally wherein the neural network is a convolutional neural network.

109-114. (canceled)

115. The computer program product of claim 107, wherein the oligonucleotide generating network and the biological activity network are deployed from individual training machine learning networks, optionally wherein the oligonucleotide generating network and the biological activity network are trained using a learning method individually selected from the group consisting of unsupervised learning, supervised learning, semi-supervised learning, reinforcement learning, transfer learning, incremental learning, curriculum learning, learning to learn, contrastive learning, and any combination thereof.

116. (canceled)

117. The computer program product of claim 89, wherein the oligonucleotide generating network comprises an objective network;
optionally wherein the objective network generates a target interaction score between the one or more target binding oligonucleotides and the one or more target nucleic acid sequences;
optionally wherein the objective network generates a non-target interaction score between the one or more target binding oligonucleotides, the one or more target nucleic acid sequences, and one or more non-target sequences;
optionally wherein the objective network comprises a logistic regression model;
optionally wherein the objective network comprises an optimizer;
optionally further comprising first training the oligonucleotide generating network with a target interaction score, non-interaction score, or both;
optionally wherein the processing step further comprises processing a target interaction score, non-interaction score, or both and the generating step further comprises generating one or more engineered target binding oligonucleotides with a corresponding target interaction score, non-interaction score, or both; and
optionally further comprising training the objective network with the one or more engineered target binding oligonucleotides with the corresponding target interaction score, non-interaction score, or both.
